(19) United States
(12) Patent Application Publication
(10) Pub. No.: US 2025/0188543 A1
(43) Pub. Date: Jun. 12, 2025

(54) METHODS FOR METHYLATION ANALYSIS OF CELL-FREE DNA

(71) Applicant: Hepta Bio, Inc., Redwood City, CA (US)

(72) Inventors: Hamed Amini, Redwood City, CA (US); Soheil Damangir, Mountain View, CA (US)

(21) Appl. No.: 19/056,221

(22) Filed: Feb. 18, 2025

Related U.S. Application Data
(63) Continuation of application No. PCT/US2024/011793, filed on Jan. 17, 2024.
(60) Provisional application No. 63/439,716, filed on Jan. 18, 2023.

(51) Int. Cl. C12Q 1/6883

(52) U.S. Cl. CPC C12Q 1/6883; C12Q 2600/112; C12Q 2600/154

(57) ABSTRACT

In an aspect, the present disclosure provides a method comprising (a) providing a cell-free deoxyribonucleic acid (cfDNA) sample derived from a subject; and (b) sequencing the cfDNA sample or a derivative thereof to determine a methylation pattern or a methylation level of DNA molecules of the cfDNA sample.

### CROSS-REFERENCE

This application is a continuation of International Application No. PCT/US2024/011793, filed Jan. 17, 2024, which claims the benefit of U.S. Provisional Application No. 63/439,716, filed Jan. 18, 2023, each of which is incorporated herein by reference in its entirety.

### BACKGROUND

Liver disease may have various pathologies, such as infections, inherited conditions, obesity, and alcohol misuse. Blood testing may be used to measure levels of enzyme biomarkers in the blood. Liver function tests, such as the international normalized ratio (INR), may be used to assess the degree of coagulopathy, an indicator of liver dysfunction. Imaging tools, such as ultrasound, magnetic resonance imaging (MRI), or computed tomography (CT), may be used to visualize signs of damage, scarring, or tumors in the liver.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### SUMMARY

Liver biopsy may be a current gold standard for evaluating liver fibrosis in patients with fatty liver disease. However, inherent risks and invasiveness of biopsy evaluations may limit widespread use. Improved diagnostic tools for the detection of liver disease may be essential for effective disease management treatment.

Recognizing the needs for improved diagnostic tools for the detection of liver disease, the present disclosure provides methods, systems, and kits for identifying or monitoring liver disease by processing cell-free biological samples obtained from or derived from subjects. Cell-free biological samples (e.g., plasma samples) obtained from subjects may be analyzed to identify liver disease, which may include, e.g., measuring a presence, absence, or relative assessment of the liver disease. Such subjects may include subjects having one or more liver diseases and subjects not having the one or more liver diseases. Liver diseases may include, for example, alcoholic fatty liver disease (AFLD), alcohol-related liver disease (ALD), metabolic and alcohol-related/associated liver disease (MetALD), non-alcoholic fatty liver disease (NAFLD), non-alcoholic steatohepatitis (NASH), steatotic liver disease (SLD), metabolic dysfunction-associated fatty liver disease (MAFLD), metabolic dysfunction-associated steatotic liver disease (MASLD), metabolic dysfunction-associated steatohepatitis (MASH), cryptogenic steatotic liver disease (cryptogenic SLD), hepatitis, cancer (e.g., hepatocellular carcinoma or hepatobiliary cancer), and cirrhosis.

In an aspect, the present disclosure provides a method for identifying whether a subject has or is at an increased risk of developing a liver disease, comprising: (a) providing a cell-free deoxyribonucleic acid (cfDNA) sample derived from the subject; (b) assaying the cfDNA sample or a derivative thereof to determine a methylation pattern or a methylation level of DNA molecules of the cfDNA sample; (c) processing the methylation pattern or the methylation level using a trained machine learning (ML) algorithm to generate an output indicative of whether the cfDNA sample is positive for the liver disease; and (d) based at least in part on the output, generating an electronic report that is indicative of the subject having or being at the increased risk of developing the liver disease.

In another aspect, the present disclosure provides a method for monitoring a liver disease in a subject, comprising: (a) providing a cell-free deoxyribonucleic acid (cfDNA) sample derived from the subject; (b) assaying the cfDNA sample or a derivative thereof to determine a methylation pattern or a methylation level of DNA molecules of the cfDNA sample; (c) processing the methylation pattern or the methylation level using a trained ML algorithm to generate an output indicative of whether the cfDNA sample is positive for the liver disease; and (d) based at least in part on the output, generating an electronic report that is indicative of progression of the liver disease in the subject.

In another aspect, the present disclosure provides a method for identifying a liver disease prognosis of a subject having or is at an increased risk of developing a liver disease, comprising: (a) providing a cell-free deoxyribonucleic acid (cfDNA) sample derived from the subject; (b) assaying the cfDNA sample or a derivative thereof to determine a methylation pattern or a methylation level of DNA molecules of the cfDNA sample; (c) processing the methylation pattern or the methylation level using a trained ML algorithm to generate an output indicative of whether the cfDNA sample is positive for the liver disease; and (d) based at least in part on the output, generating an electronic report that is indicative of the prognosis of the subject having or is at the increased risk of developing the liver disease.

In another aspect, the present disclosure provides a method for identifying a treatment for a subject having or is at an increased risk of developing a liver disease, comprising: (a) providing a cell-free deoxyribonucleic acid (cfDNA) sample derived from the subject; (b) assaying the cfDNA sample or a derivative thereof to determine a methylation pattern or a methylation level of DNA molecules of the cfDNA sample; (c) processing the methylation pattern or the methylation level using a trained ML algorithm to generate an output indicative of whether the cfDNA sample is positive for the liver disease; and (d) based at least in part on the output, generating an electronic report that is indicative of the treatment for the subject having or is at the increased risk of developing the liver disease.

In another aspect, the present disclosure provides a method for determining a treatment response for a subject having or is at an increased risk of developing a liver disease, comprising: (a) providing a cell-free deoxyribonucleic acid (cfDNA) sample derived from the subject; (b) assaying the cfDNA sample or a derivative thereof to determine a methylation pattern or a methylation level of DNA molecules of the cfDNA sample; (c) processing the methylation pattern or the methylation level using a trained ML algorithm to generate an output indicative of whether the cfDNA sample is positive for the liver disease; and (d) based at least in part on the output, generating an electronic report that is indicative of the treatment response for the subject having or is at the increased risk of developing the liver disease.

In some embodiments, the assaying comprises identifying the methylation pattern and the methylation level of the DNA molecules of the cfDNA sample, wherein the methylation pattern and the methylation level are processed using the trained ML algorithm.

In some embodiments, the assaying comprises sequencing.

In some embodiments, the method further comprises, prior to the sequencing, processing the DNA molecules of the cfDNA sample with a reaction mixture comprising enzymes for methylation-aware sequencing.

In some embodiments, the method further comprises, prior to the sequencing, processing the DNA molecules of the cfDNA sample with a reaction mixture comprising bisulfite.

In some embodiments, the assay comprises amplification.

In some embodiments, the amplification comprises polymerase chain reaction (PCR).

In some embodiments, the cfDNA sample is obtained or derived from a plasma sample, a serum sample, a urine sample, a saliva sample, or a liver tissue sample.

In some embodiments, the method further comprises fractionating a whole blood sample derived from the subject to provide the cfDNA sample.

In some embodiments, (a) comprises subjecting the cfDNA sample to conditions that are sufficient to isolate, enrich, or extract a set of DNA molecules, and wherein (b) comprises assaying the DNA molecules.

In some embodiments, (b) comprises using nucleic acid primers or probes to selectively enrich the set of DNA molecules corresponding to a panel of one or more genomic regions.

In some embodiments, the one or more genomic regions are selected from the group consisting of genes listed in TABLE 1.

In some embodiments, the nucleic acid primers or probes have sequence complementarity with nucleic acid sequences of the panel of the one or more genomic regions.

In some embodiments, the cfDNA sample is assayed without nucleic acid isolation, enrichment, or extraction.

In some embodiments, the subject is asymptomatic for the liver disease.

In some embodiments, the output is indicative of whether the cfDNA sample is positive for the liver disease with an accuracy of at least 50%.

In some embodiments, the accuracy is determined by calculating a percentage of independent samples that are correctly identified as having or not having the liver disease.

In some embodiments, the output is indicative of whether the cfDNA sample is positive for the liver disease with a clinical sensitivity of at least 50%.

In some embodiments, the clinical sensitivity is at least 50%.

In some embodiments, the output is indicative of whether the cfDNA sample is positive for the liver disease with a clinical specificity of at least 50%.

In some embodiments, the clinical specificity is at least 50%.

In some embodiments, the output is indicative of whether the cfDNA sample is positive for the liver disease with a positive predictive value of at least 50%.

In some embodiments, the output is indicative of whether the cfDNA sample is positive for the liver disease with a negative predictive value of at least 50%.

In some embodiments, the output is indicative of whether the cfDNA sample is positive for the liver disease with an area under the receiver operating characteristic (AUROC) of at least 0.50.

In some embodiments, the output is indicative of whether the cfDNA sample is positive for the liver disease with a positive likelihood ratio of at least about 1.3.

In some embodiments, the output is indicative of whether the cfDNA sample is negative for the liver disease with a negative likelihood ratio of at most about 0.75.

In some embodiments, the liver disease is early stage liver disease.

In some embodiments, the liver disease is advanced stage liver disease.

In some embodiments, the liver disease is non-alcoholic steatohepatitis (NASH) or metabolic dysfunction-associated steatohepatitis (MASH).

In some embodiments, the liver disease is fibrosis.

In some embodiments, the liver disease is cirrhosis.

In some embodiments, the liver disease is hepatocellular carcinoma (HCC).

In some embodiments, the liver disease is a hepatobiliary cancer, including, e.g., cholangiocarcinoma, angiosarcoma, gallbladder cancer, or undifferentiated embryonal sarcoma of the liver (UESL).

In some embodiments, the liver disease is viral hepatitis.

In some embodiments, the liver disease is non-alcoholic fatty liver disease (NAFLD) or metabolic dysfunction-associated steatotic liver disease (MASLD).

In some embodiments, the liver disease is non-alcoholic fatty liver (NAFL) or steatosis.

In some embodiments, the liver disease is metabolic dysfunction-associated fatty liver disease (MAFLD).

In some embodiments, the liver disease is alcohol-related liver disease (ALD).

In some embodiments, the liver disease is metabolic and alcohol-related/associated liver disease (MetALD).

In some embodiments, the method further comprises, based at least in part on the output, providing the subject with a therapeutic intervention for the liver disease.

In some embodiments, the liver disease is NASH, and wherein the therapeutic intervention is vitamin E supplementation, a weight loss agent, an anti-hypertensive agent, an anti-diabetic agent, a cholesterol-lowering agent, an exercise regimen, a diet regimen, or bariatric surgery.

In some embodiments, the liver disease is NASH, and wherein the therapeutic intervention is a GLP1 (glucagon-like peptide-1) receptor agonist, a FGF (fibroblast growth factor) analog, a THR (thyroid hormone receptor) agonist, a SCD-1 (stearoyl-coenzyme A desaturase 1) inhibitor, a FAS (fatty acid synthase) inhibitor, a FXR (farnesoid X receptor) agonist, an ACC (acetyl-CoA carboxylase) inhibitor, a PPAR (peroxisome proliferator-activated receptor) agonist, a targeted genetic modifier, including, e.g., PNPLA3 or HSD17B13, a LOXL2 (lysyl oxidase-like 2) inhibitor, a pan-cyclophilin inhibitor, a pan-caspase inhibitor, a chemokine receptor (e.g., CCR2/CCR5) inhibitor, a galactin-3 inhibitor, a mitochondrial uncoupler or uncoupling agent, a structurally engineered fatty acid, or a combination thereof.

In some embodiments, the liver disease is NAFLD, and wherein the therapeutic intervention is vitamin E supplementation, a weight loss agent, an anti-hypertensive agent, an anti-diabetic agent, a cholesterol-lowering agent, an exercise regimen, a diet regimen, bariatric surgery, or a combination thereof.

In some embodiments, the liver disease is NAFLD, and wherein the therapeutic intervention is a GLP1 receptor agonist, a FGF analog, a THR agonist, a SCD-1 inhibitor, a FAS inhibitor, a FXR agonist, an ACC inhibitor, a PPAR agonist, a targeted genetic modifier, including, e.g., PNPLA3 or HSD17B13, a LOXL2 (lysyl oxidase-like 2) inhibitor, a pan-cyclophilin inhibitor, a pan-caspase inhibitor, a chemokine receptor (e.g., CCR2/CCR5) inhibitor, a galactin-3 inhibitor, a mitochondrial uncoupler or uncoupling agent, a structurally engineered fatty acid, or a combination thereof.

In some embodiments, the method further comprises, based at least in part on the output, monitoring the subject for the liver disease at two or more time points.

In some embodiments, the method further comprises, determining a likelihood or risk score of the subject having or being at the increased risk of having the liver disease.

In some embodiments, the method further comprises, determining a molecular subtype, a grade, a stage, or a severity of the liver disease.

In some embodiments, the method further comprises, determining a prognosis of the liver disease.

In some embodiments, the method further comprises, determining eligibility of the subject as a liver transplant donor or a liver transplant recipient.

In some embodiments, the subject is determined to be eligible as the liver transplant donor if the subject is not identified as having or being at the increased risk of developing the liver disease.

In some embodiments, the subject is determined to be eligible as the liver transplant recipient if the subject is identified as having or being at the increased risk of developing the liver disease.

In some embodiments, the trained ML algorithm is trained with a set of independent samples associated with a presence or increased risk of the liver disease.

In some embodiments, the trained ML algorithm is trained with a first set of independent samples associated with a presence or increased risk of the liver disease and a second set of independent samples associated with an absence or no increased risk of the liver disease.

In some embodiments, (c) further comprises using the trained ML algorithm or another trained algorithm to process a set of clinical health data of the subject.

In some embodiments, the clinical health data comprises one or more quantitative measures selected from the group consisting of age, weight, height, body mass index (BMI), blood pressure, heart rate, aspartate aminotransferase (AST) levels, alanine transaminase (ALT) levels, gamma-glutamyl transferase (GGT), platelet count, triglyceride levels, glycated hemoglobin (HbA1c) levels, creatinine levels, insulin levels, prothrombin time, haptoglobin levels, and glucose levels.

In some embodiments, the clinical health data comprises one or more categorical measures selected from the group consisting of race, ethnicity, history of medication or other clinical treatment, history of alcohol use, daily activity or fitness level, genetic test results, blood test results, and imaging results.

In some embodiments, the trained ML algorithm comprises a supervised ML algorithm.

In some embodiments, the supervised ML algorithm comprises a classifier or a regression.

In some embodiments, the supervised ML algorithm comprises a deep learning algorithm, a support vector machine (SVM), a neural network, a random forest, a linear regression, or a logistic regression.

In some embodiments, the methylation pattern or the methylation level is represented by parameters of a distribution, sufficient statistics, or a near sufficient statistics.

In another aspect, the present disclosure provides method for determining whether a subject has or is at an increased risk of developing a liver disease, comprising: (a) providing a cell-free nucleic acid sample derived from the subject; (b) assaying the cell-free nucleic acid sample or a derivative thereof to determine a methylome of the cell-free nucleic acid sample;

and (c) processing the methylome using a trained machine learning (ML) algorithm to determine whether the subject has or is at the increased risk of developing the liver disease, wherein the determining has a sensitivity of at least about 70% and a specificity of at least about 70%.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example workflow of a method for identifying or monitoring a liver disease state of a subject.

FIG. 2 illustrates a computer system that is programmed or otherwise configured to implement methods provided herein.

FIG. 3 illustrates a schematic of an example training data.

FIG. 4 illustrates score distributions of cfDNA methylation data that distinguish non-alcoholic steatohepatitis (NASH) samples from non-NASH (healthy) samples.

FIG. 5 illustrates score distributions of cfDNA methylation data that distinguish at-risk NASH samples from non-at-risk NASH samples, with at-risk NASH defined as individuals with NASH and fibrosis of stage 2 or higher.

FIG. 6 illustrates score distributions of cfDNA methylation data that distinguish NASH samples with cirrhosis from NASH samples without cirrhosis.

FIG. 7 illustrates score distributions of cfDNA methylation data that distinguish early stage NASH samples, late stage NASH samples, and non-NASH (healthy) samples.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Differential patterns in nucleic acid molecules may be useful for the detection or stratification of liver disease. Provided herein are methods and systems for assaying nucleic acids for the detection or stratification of liver disease. For example, methylation patterns of circulating deoxyribonucleic acid (DNA) may be detected in human plasma and used to stratify liver fibrosis severity in patients with NAFLD.

Liver disease refers to several conditions that affect and damage the liver. There are four main stages of liver disease: 1) inflammation; 2) fibrosis; 3) cirrhosis; and 4) liver failure or liver cancer. Early stage liver disease may be characterized by inflammation or enlargement of the liver or fibrosis. Over time, liver disease can cause cirrhosis (scarring). As more scar tissue replaces healthy liver tissue, the liver can no longer function properly. When left untreated, liver disease can lead to more severe conditions, such as liver failure and cancer. Advanced stage liver disease, also referred to as end-stage liver disease or late-stage liver disease, may be characterized by irreversible cirrhosis, liver failure, and stage 4 hepatitis C. Steatotic liver disease (SLD) encompasses all the various etiologies of steatosis.

Non-alcoholic fatty liver disease (NAFLD) is a common chronic pathology associated with progressive histological alterations of the hepatic parenchyma. These NAFLD-associated changes range from a simple fat accumulation in hepatocytes, also referred to as hepatic steatosis or fatty liver, to a more severe histology characterized by liver cell injury, fibrosis, and inflammation, which are hallmarks of non-alcoholic steatohepatitis (NASH). NASH is also referred to as metabolic dysfunction-associated steatohepatitis (MASH).

Non-alcoholic fatty liver disease (NAFLD) is a common cause of chronic liver pathology worldwide. The prevalence of NAFLD strongly correlates with the increasing incidence of diabetes, obesity, and metabolic syndrome in the general population. Simple steatosis, the earliest stage of NAFLD, is often non-progressive and remains asymptomatic. Proper modifications in the lifestyle and diet at this early stage may reverse the affected liver into the healthy state. The potential of simple steatosis to progress into severe fibrotic stages and facilitate carcinogenesis necessitates timely NAFLD detection and risk stratification.

NAFLD is also referred to as metabolic dysfunction-associated steatosis liver disease (MASLD). MASLD encompasses patients who have hepatic steatosis and have at least one of five cardiometabolic risk factors. Another category, outside pure MASLD, termed metabolic and alcohol-related/associated liver disease (MetALD), refers to patients with MASLD who consume greater amounts of alcohol per week (e.g., 140 g/week and 210 g/week for females and males, respectively). Liver disease patients with no metabolic parameters and no known cause can be referred to as cryptogenic steatosis liver disease (cryptogenic SLD). The methods described herein may be used to identify, stratify, or distinguish any liver disease types or subtypes, e.g., described herein and in Rinella et al. Hepatology 78(6): p 1966-1986, December 2023 DOI: 10.1097/HEP.0000000000000520, which is incorporated herein by reference in its entirety.

Extracellular circulating nucleic acids found in biological fluids including blood may serve as promising non-invasive biomarkers for liver disease. For example, epigenetic signatures of circulating cfDNA, such as methylation patterns, may be useful for detecting presence of disease and monitoring disease progression. Intracellular miRNAs normally participate in the regulation of gene expression, but after released by apoptotic cells, miRNAs may remain highly stable in the extracellular environment for prolonged periods. Thus, circulating nucleic acid profiles may reflect the pathogenic processes in the body's tissues and organs to enable highly sensitive, non-invasive detection of liver diseases.

### Definitions

As used herein, the term “nucleic acid” generally refers to a polymeric form of nucleotides of any length, either deoxyribonucleotides (dNTPs) or ribonucleotides (rNTPs), or analogs thereof. Nucleic acids may have any three-dimensional structure, and may perform any function, known or unknown. Non-limiting examples of nucleic acids include DNA, ribonucleic acid (RNA), coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), microRNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non-nucleotide components. A nucleic acid may be further modified after polymerization, such as by conjugation or binding with a reporter agent.

The terms “nucleic acid molecule,” “nucleic acid sequence,” “nucleic acid fragment,” “oligonucleotide” and “polynucleotide,” as used herein, generally refer to a polynucleotide, such as deoxyribonucleotides (DNA) or ribonucleotides (RNA), or analogs and/or combinations thereof (e.g., mixture of DNA and RNA). A nucleic acid molecule may have various lengths. A nucleic acid molecule can have a length of at least about 5 bases, 10 bases, 20 bases, 30 bases, 40 bases, 50 bases, 60 bases, 70 bases, 80 bases, 90, 100 bases, 110 bases, 120 bases, 130 bases, 140 bases, 150 bases, 160 bases, 170 bases, 180 bases, 190 bases, 200 bases, 300 bases, 400 bases, 500 bases, 1 kilobase (kb), 2 kb, 3, kb, 4 kb, 5 kb, 10 kb, or 50 kb or it may have any number of bases between any two of the aforementioned values. An oligonucleotide is typically composed of a specific sequence of four nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

The terms “nucleic acid molecule,” “nucleic acid sequence,” “nucleic acid fragment,” “oligonucleotide” and “polynucleotide,” as used herein, generally refer to a polynucleotide, such as deoxyribonucleotides (DNA) or ribonucleotides (RNA), or analogs and/or combinations thereof (e.g., mixture of DNA and RNA). A nucleic acid molecule may have various lengths. A nucleic acid molecule can have a length of at least 5 bases, at least 10 bases, at least 20 bases, at least 30 bases, at least 40 bases, at least 50 bases, at least 60 bases, at least 70 bases, at least 80 bases, at least 90, at least 100 bases, at least 110 bases, at least 120 bases, at least 130 bases, at least 140 bases, at least 150 bases, at least 160 bases, at least 170 bases, at least 180 bases, at least 190 bases, at least 200 bases, at least 300 bases, at least 400 bases, at least 500 bases, at least 1 kilobase (kb), at least 2 kb, at least 3, kb, at least 4 kb, at least 5 kb, at least 10 kb, at least 50 kb, or any number of bases between any two of the aforementioned values. An oligonucleotide is typically composed of a specific sequence of four nucleotide bases: adenine (A); cytosine (C); guanine (G); and thymine (T) (uracil (U) for thymine (T) when the polynucleotide is RNA). Thus, the terms “nucleic acid molecule,” “nucleic acid sequence,” “nucleic acid fragment,” “oligonucleotide” and “polynucleotide” are at least in part intended to be the alphabetical representation of a polynucleotide molecule. Alternatively, the terms may be applied to the polynucleotide molecule itself. This alphabetical representation can be input into databases in a computer having a central processing unit and/or used for bioinformatics applications such as functional genomics and homology searching. Oligonucleotides may include one or more nonstandard nucleotide(s), nucleotide analog(s) and/or modified nucleotides.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogs thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “target” generally refers to a genomic region within a marker gene or marker region. As used herein, the term “reference” generally refers to a sample obtained or derived from a subject who is diagnosed with liver disease or who has received a negative clinical indication of liver disease (e.g., a healthy or control subject without a liver disease).

As used herein, the terms “locus” or “region” are generally interchangeable and refer to a specific genomic region on the genome represented by chromosome number, start position, and end position.

As used herein, the term “subject,” generally refers to an entity or a medium that has testable or detectable genetic information. A subject can be a person or individual, such as a patient. A subject can be a vertebrate, such as, for example, a mammal. Non-limiting examples of mammals include murines, simians, humans, farm animals, sport animals, and pets.

As used herein, the term “sample” generally refers to a biological sample, e.g., obtained or derived from a subject. The samples may be obtained from tissue and/or cells or from the environment of tissue and/or cells. The samples may be cell-free biological samples or substantially cell-free biological samples, or may be processed or fractionated to produce cell-free biological samples. For example, cell-free biological samples may include cell-free ribonucleic acid (cfRNA), cell-free deoxyribonucleic acid (cfDNA), cell-free fetal DNA (cffDNA), plasma, serum, urine, saliva, amniotic fluid, and derivatives thereof. Cell-free biological samples may be obtained or derived from subjects using an ethylenediaminetetraacetic acid (EDTA) collection tube, a cell-free RNA collection tube, or a cell-free DNA collection tube. Cell-free biological samples may be derived from whole blood samples by fractionation. In some embodiments, biological samples or derivatives thereof may contain cells. For example, a biological sample may be a blood sample or a derivative thereof (e.g., blood collected by a collection tube or blood drops), a liver tissue sample, a vaginal sample (e.g., a vaginal swab), or a cervical sample (e.g., a cervical swab). In some examples, the sample may comprise, be obtained or derived from, a tissue biopsy (e.g., a liver tissue biopsy), a cell biopsy, blood (e.g., whole blood), blood plasma, serum, bone marrow, cerebral spinal fluid, pleural fluid, saliva, stool, urine, extracellular fluid, dried blood spots, cultured cells, culture media, discarded tissue, plant matter, synthetic proteins, bacterial and/or viral samples, fungal tissue, archaea, or protozoans. The sample may have been isolated from the source prior to collection. Non-limiting examples include a fingerprint, saliva, urine, blood, stool, semen, or other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some embodiments, the primary source is homogenized prior to further processing. The sample may be filtered or centrifuged to remove buffy coat, lipids, or particulate matter. The sample may also be purified or enriched for nucleic acids, or may be treated with RNases or DNases. The sample may contain tissues and/or cells that are intact, fragmented, or partially degraded.

The sample may be obtained from a subject having or suspected of having a disease or disorder, and the subject may or may not have had a diagnosis of the disease or disorder. The subject may be in need of a second opinion. The disease or disorder may be an infectious disease, an immune disorder or disease, a cancer, a genetic disease, a degenerative disease, a lifestyle disease, or an injury. The infectious disease may be caused by bacteria, viruses, fungi, and/or parasites. The cancer may be hepatocellular carcinoma (HCC) or a hepatobiliary cancer, including, e.g., cholangiocarcinoma, angiosarcoma, gallbladder cancer, or undifferentiated embryonal sarcoma of the liver (UESL).

Components of the sample (including nucleic acids) may be tagged, e.g., with identifiable tags, to allow for multiplexing of samples. Some non-limiting examples of identifiable tags include: fluorophores, magnetic nanoparticles, and nucleic acid barcodes. Fluorophores may include fluorescent proteins such as GFP, YFP, RFP, eGFP, mCherry, tdtomato, FITC, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Pacific Blue, Coumarin, BODIPY FL, Pacific Green, Oregon Green, Cy3, Cy5, Pacific Orange, TRITC, Texas Red, Phycoerythrin, Allophcocyanin, or other fluorophores. One or more barcode tags may be attached (e.g., by coupling or ligating) to cell-free nucleic acids (e.g., cfDNA) in the sample prior to sequencing. The barcodes may uniquely tag the cfDNA molecules in a sample. Alternatively, the barcodes may non-uniquely tag the cfDNA molecules in a sample. The barcode(s) may non-uniquely tag the cfDNA molecules in a sample such that additional information obtained from the cfDNA molecule (e.g., at least a portion of the endogenous sequence of the cfDNA molecule), obtained in combination with the non-unique tag, may function as a unique identifier for (e.g., to uniquely identify against other molecules) the cfDNA molecule in a sample. For example, cfDNA sequence reads having unique identity (e.g., from a given template molecule) may be detected based at least in part on sequence information comprising one or more contiguous-base regions at one or both ends of the sequence read, the length of the sequence read, and/or the sequence of the attached barcodes at one or both ends of the sequence read. DNA molecules may be uniquely identified without tagging by partitioning a DNA (e.g., cfDNA) sample into many (e.g., at least about 50, at least about 100, at least about 500, at least about 1 thousand, at least about 5 thousand, at least about 10 thousand, at least about 50 thousand, or at least about 100 thousand) different discrete subunits (e.g., partitions, wells, or droplets) prior to amplification, such that amplified DNA molecules can be uniquely resolved and identified as originating from their respective individual input molecules of DNA.

Any number of samples may be multiplexed. For example, a multiplexed analysis may contain at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, or more samples. The identifiable tags may provide a way to interrogate each sample as to its origin, or may direct different samples to segregate to different areas or a solid support.

Any number of samples may be mixed prior to analysis without tagging or multiplexing. For example, a multiplexed analysis may contain at least about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, or more samples. Samples may be multiplexed without tagging using a combinatorial pooling design in which samples are mixed into pools in a manner that allows signal from individual samples to be resolved from the analyzed pools using computational demultiplexing.

The samples may be enriched prior to sequencing. For example, the cfDNA molecules may be selectively enriched or non-selectively enriched for one or more regions from the subject's genome or transcriptome. For example, the cfDNA molecules may be selectively enriched for one or more regions from the subject's genome or transcriptome by targeted sequence capture (e.g., using a panel), selective amplification, or targeted amplification. As another example, the cfDNA molecules may be non-selectively enriched for one or more regions from the subject's genome or transcriptome by universal amplification. In some embodiments, amplification comprises universal amplification, whole genome amplification, or non-selective amplification. The cfDNA molecules may be size selected for fragments having a length in a predetermined range. For example, size selection can be performed on DNA fragments prior to adapter ligation for lengths in a range of about 40 base pairs (bp) to about 250 bp. As another example, size selection can be performed on DNA fragments after adapter ligation for lengths in a range of about 160 bp to about 400 bp.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product.” The term “reverse transcription amplification” generally refers to the generation of deoxyribonucleic acid (DNA) from a ribonucleic acid (RNA) template via the action of a reverse transcriptase. Amplification may be performed by polymerase chain reaction (PCR), which is based on using DNA polymerase to synthesize new strands of DNA complementary to the initial template strands.

As used herein, the term “polymerase chain reaction” or “PCR” generally refers to a method for increasing the concentration of a segment of a target sequence in a mixture of genomic DNA without cloning or purification. This process for amplifying the target sequence may comprise introducing a large excess of two oligonucleotide primers to the DNA mixture containing the desired target sequence, followed by a precise sequence of thermal cycling in the presence of a DNA polymerase. The two primers may be complementary to their respective strands of the double-stranded target sequence. To perform amplification, the mixture may be denatured, and the primers may be annealed to their complementary sequences within the target molecule. Following annealing, the primers may be extended with a polymerase so as to form a new pair of complementary strands. The denaturation, primer annealing, and polymerase extension can be repeated many times (e.g., denaturation, annealing and extension constitute one “cycle”; there can be numerous “cycles”) to obtain a high concentration of an amplified segment of the desired target sequence. The length of the amplified segment of the desired target sequence may be determined by the relative positions of the primers with respect to each other, and therefore, this length is a controllable parameter. By virtue of the repeating aspect of the process, the method is referred to as “polymerase chain reaction” or “PCR”. Because the desired amplified segments of the target sequence become the predominant sequences (in terms of concentration) in the mixture, the amplified segments may be referred to as “PCR amplified,” “PCR products,” or “amplicons.”

As used herein, the term “methylation” refers to 5-methylcytosine (5mC) or 5-hydroxymethylcytosine (5hmC), including cytosine residues that are part of the sequence CG, also denoted as CpG dinucleotides. Some CG dinucleotides in the human genome are methylated, while others are not. In addition, methylation can be cell-specific and tissue-specific, such that a specific CG dinucleotide can be methylated in a certain cell and at the same time unmethylated in a different cell, or methylated in a certain tissue and at the same time unmethylated in different tissues. DNA methylation can be an important regulator of gene transcription. Aberrant DNA methylation patterns, both hypermethylation and hypomethylation, as compared to normal tissue, may be associated with a large number of human malignancies. In some embodiments, 5hmC residues of a sequence may be subjected to glucosylation prior to subsequent bisulfite treatment, bisulfite-free enzymatic treatment, or methylation-sensitive restriction enzyme digestion. For example, the glucosylation may be performed using a glucosyltransferase.

As used herein, the terms “methylation state,” “methylation status,” and “methylation profile” generally refer to the presence of absence of one or more methylated nucleotide bases in the nucleic acid molecule. For example, a nucleic acid molecule (e.g., DNA molecule) containing a methylated cytosine is considered methylated (e.g., the methylation state of the nucleic acid molecule is methylated). A nucleic acid molecule that does not contain any methylated nucleotides is considered unmethylated.

As used herein, the term “DNA template” generally refers to the sample DNA that contains the target sequence. At the beginning of the reaction, high temperature is applied to the original double-stranded DNA molecule to separate the strands from each other.

As used herein, the term “primer” generally refers to a short piece of single-stranded DNA that are complementary to the DNA template. The polymerase begins synthesizing new DNA from the end of the primer.

As used herein, the term “sensitivity” or “clinical sensitivity” generally refers to the percentage of a set of diseased samples for which a positive diagnostic result is obtained. For example, such diseased samples may be analyzed to detect a DNA methylation value that is above a threshold value that distinguishes between disease (e.g., liver disease) and non-disease (e.g., healthy or control) samples. In some embodiments, a positive is defined as a histology-confirmed disease that reports a DNA methylation value above a threshold value (e.g., the range associated with disease), and a false negative is defined as a histology-confirmed disease that reports a DNA methylation value below the threshold value (e.g., the range associated with no disease). The value of sensitivity may reflect the probability that a DNA methylation measurement for a given marker obtained from a diseased sample falls in the range of disease-associated measurements. The clinical relevance of the calculated sensitivity value may represent an estimation of the probability that a given marker can detect or predict the presence of a clinical condition when applied to a subject having the clinical condition.

As used herein, the term “specificity” or “clinical specificity” generally refers to the percentage of a set of non-diseased samples for which a negative diagnostic result is obtained. For example, such non-diseased samples may be analyzed to detect a DNA methylation value below a threshold value that distinguishes between diseased (e.g., liver disease) and non-diseased (e.g., non-liver disease) samples. In some embodiments, a negative is defined as a histology-confirmed non-disease sample that reports a DNA methylation value below the threshold value (e.g., the range associated with no disease) and a false positive is defined as a histology-confirmed non-disease sample that reports a DNA methylation value above the threshold value (e.g., the range associated with disease). The value of specificity may reflect the probability that a DNA methylation measurement for a given marker obtained from a non-liver disease (e.g., healthy or control) sample falls in the range of non-disease associated measurements. The clinical relevance of the calculated specificity value may represent an estimation of the probability that a given marker can detect or predict the absence of a clinical condition when applied to a subject not having the clinical condition.

As used herein, the term “AUC” or “AUROC” generally refers to the area under a Receiver Operating Characteristic (ROC) curve. The ROC curve may be a plot of the true positive rate (TPR) against the false positive rate (FPR) for a plurality of different possible thresholds or cut points of a diagnostic test, thereby illustrating the trade-off between sensitivity and specificity depending on the selected cut point (e.g., any increase in sensitivity is accompanied by a decrease in specificity). The area under an ROC curve (AUC) can be a measure for the accuracy of a diagnostic test (e.g., the larger the area, the more accurate the diagnosis), with an optimal value of 1. In comparison, a random test may have an ROC curve lying on the diagonal with an AUC of 0.5 (e.g., representing a random or worthless test).

### Methods of the Disclosure

Current diagnostic tools for liver disease may be inaccessible and incomplete. Blood testing may be used to measure levels of enzyme biomarkers in the blood. Liver function tests, such as the international normalized ratio (INR), may be used to assess the degree of coagulopathy, an indicator of liver dysfunction. Imaging tools, such as ultrasound, MRI, or CT, may be used to visualize signs of damage, scarring, or tumors in the liver. Liver biopsy is a current gold standard for evaluating liver fibrosis in patients with fatty liver disease. However, inherent risks and invasiveness of biopsy evaluations limit widespread use. Therefore, there is an urgent clinical need for accurate, affordable, and non-invasive diagnostic methods for detection and monitoring of liver disease toward effective disease management treatment.

The present disclosure provides methods, systems, and kits for identifying or monitoring liver disease by processing cell-free biological samples obtained from or derived from subjects. Cell-free biological samples (e.g., plasma samples) obtained from subjects may be analyzed to identify liver disease, which may include, e.g., measuring a presence, absence, or relative assessment of the liver disease. Such subjects may include subjects having one or more liver diseases and subjects not having the one or more liver diseases. Liver diseases may include, for example, alcoholic or non-alcoholic fatty liver disease, non-alcoholic steatohepatitis, hepatitis, cancer (e.g., hepatocellular carcinoma), and cirrhosis.

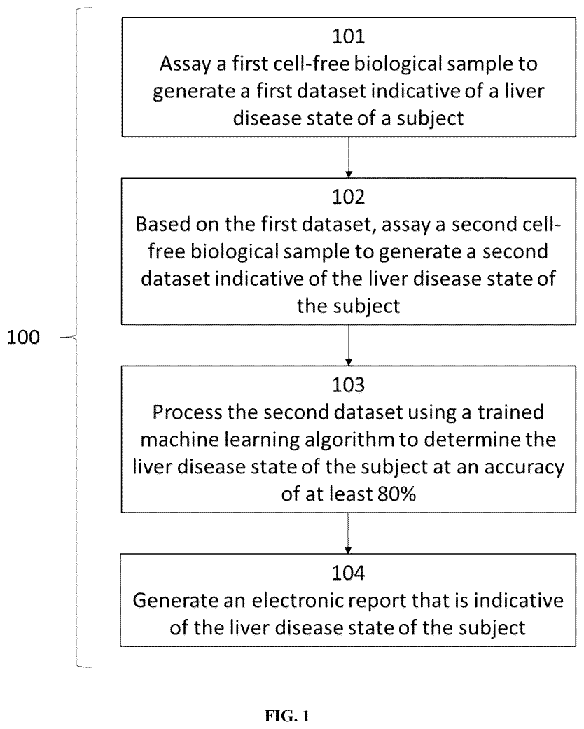

FIG. 1 illustrates an example workflow of a method for identifying or monitoring a liver disease state of a subject, in accordance with embodiments disclosed herein. In an aspect, the present disclosure provides a method 100 for identifying or monitoring a liver disease state of a subject. The method 100 may comprise assaying by a first assay a first cell-free biological sample derived from the subject to generate a first dataset (operation 101). Next, based at least in part on the first dataset generated, the method 100 may optionally comprise assaying by a second assay (e.g., a different assay from the first assay) a second cell-free biological sample derived from the subject to generate a second dataset indicative of the liver disease state at a specificity greater than the first dataset (operation 102). For example, DNA molecules extracted from a second cell-free plasma sample may be sequenced to generate a set of sequence reads indicative of a liver disease state of the subject. In some embodiments, a first cell-free biological sample is obtained from a subject at a first time point for processing with a first assay. Then, optionally a second cell-free biological sample is obtained from the same subject at a second time point for processing with a second assay. In some embodiments, a cell-free biological sample can be obtained from a subject and then aliquoted to produce a first cell-free biological sample and a second cell-free biological sample, which can then be processed with a first assay and a second assay, respectively. Next, a trained machine learning algorithm may be used to process the first dataset and/or the second dataset to determine the liver disease state of the subject (operation 103). The trained machine learning algorithm may be configured to identify the liver disease at an accuracy of at least about 80% over 50 independent samples. A report may then be electronically generated that is indicative of (e.g., identifies or provides an indication of) presence or susceptibility of the liver disease of the subject (operation 104).

Cell-free biological samples may be obtained from a subject having a liver disease state (e.g., a liver disease or condition), from a subject that is suspected of having a liver disease state, or from a subject that does not have or is not suspected of having the liver disease state. The disease or disorder may be a disease or disorder affecting the liver. Non-limiting examples of such diseases or disorders include fatty liver disease, alcoholic fatty liver disease, non-alcoholic fatty liver disease, steatohepatitis, non-alcoholic steatohepatitis, hepatitis (e.g., hepatitis A, hepatitis B, or hepatitis C), liver cancer (e.g., hepatocellular carcinoma), hepatobiliary cancer, including, e.g., cholangiocarcinoma, angiosarcoma, gallbladder cancer, or undifferentiated embryonal sarcoma of the liver (UESL)), cirrhosis, hemochromatosis, Wilson disease, obesity, diabetes, hypertension, and other liver conditions disclosed herein.

The sample may be obtained before and/or after treatment of a subject having a disease or disorder. Samples may be obtained before and/or after a treatment of the subject for a disease or disorder. Samples may be obtained during a treatment or a treatment regimen. Multiple samples may be obtained from a subject to monitor the effects of a treatment over time, including beginning from prior to the onset of the treatment. Samples may be obtained from a subject to monitor abnormal tissue-specific cell death or organ transplantation.

The sample may be obtained from a subject suspected of having a disease or a disorder. The sample may be obtained from a subject experiencing unexplained symptoms, such as fatigue, nausea or vomiting, yellowing of skin or eyes (jaundice), swelling of legs or ankles, abdominal swelling (ascites), abdominal pain, itchy skin, weight gain, weight loss, aches, pains, tremors, weakness, sleepiness, or disorientation or confusion. The sample may be obtained from a subject having explained symptoms. The sample may be obtained from a subject at risk of developing a disease or disorder because of one or more factors such as familial and/or personal history, age, weight, height, body mass index (BMI), blood pressure, heart rate, aspartate aminotransferase (AST) levels, alanine transaminase (ALT) levels, gamma-glutamyl transferase (GGT), platelet count, triglyceride levels, haptoglobin levels, glucose levels, environmental exposure, lifestyle risk factors, presence of other risk factors, or a combination thereof.

The sample may be obtained from a healthy subject or individual. In some embodiments, samples may be obtained longitudinally from the same subject or individual. In some embodiments, samples acquired longitudinally may be analyzed with the goal of monitoring individual health and early detection of health issues (e.g., early diagnosis of a liver disease). In some embodiments, the sample may be collected at a home setting or at a point-of-care setting, and subsequently transported by a mail delivery, courier delivery, or other transport method prior to analysis. For example, a home user may collect a blood spot sample through a finger prick. The blood spot sample may be dried, and subsequently transported by mail delivery prior to analysis. In some embodiments, samples acquired longitudinally may be used to monitor response to stimuli expected to impact health, athletic performance, or cognitive performance. Non-limiting examples include response to a medication, dieting, and/or an exercise regimen. In some embodiments, the individual sample is multi-purpose and allows for methylation profiling to obtain clinically relevant information but may also be used for obtaining information about the individual's personal or family ancestry.

In some embodiments, a biological sample is a nucleic acid sample including one or more nucleic acid molecules. The nucleic acid molecules may be cell-free or substantially cell-free nucleic acid molecules, such as cell-free DNA (cfDNA) or cell-free RNA (cfRNA) or a mixture thereof. The nucleic acid molecules may be derived from a variety of sources including human, mammal, non-human mammal, ape, monkey, chimpanzee, reptilian, amphibian, or avian sources. Further, samples may be extracted from variety of animal fluids containing cell-free sequences, including but not limited to blood, serum, plasma, bone marrow, vitreous, sputum, stool, urine, tears, perspiration, saliva, semen, mucosal excretions, mucus, cerebral spinal fluid, pleural fluid, amniotic fluid, and lymph fluid.

The cell-free biological sample may contain one or more analytes capable of being assayed, such as cfRNA molecules suitable for assaying to generate transcriptomic data, cfDNA molecules suitable for assaying to generate genomic data, proteins suitable for assaying to generate proteomic data, metabolites suitable for assaying to generate metabolomic data, or a mixture or combination thereof. One or more such analytes (e.g., cfRNA molecules, cfDNA molecules, proteins, or metabolites) may be isolated or extracted from one or more cell-free biological samples of a subject for downstream assaying using one or more suitable assays.

After obtaining a cell-free biological sample from the subject, the sample may be processed to generate datasets indicative of a liver disease state of the subject. For example, a presence, absence, or quantitative assessment of nucleic acid molecules of the sample at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites may be indicative of a liver disease state. Processing the cell-free biological sample obtained from the subject may comprise: (i) subjecting the sample to conditions that are sufficient to isolate, enrich, or extract a plurality of nucleic acid molecules, proteins, and/or metabolites, and (ii) assaying the plurality of nucleic acid molecules, proteins, and/or metabolites to generate the dataset. In some embodiments, the quantitative measures of DNA may comprise a presence, an absence, or a degree of methylation, hypermethylation, and/or hypomethylation. Alternatively, or in combination, the quantitative measures of DNA may comprise a presence, an absence, or a degree of a variant pattern. A variant pattern can comprise a genetic mutation, a single nucleotide polymorphism (SNP), or a copy-number variation. Alternatively, or in combination, the quantitative measures of DNA may comprise a presence, an absence, or a degree of a viral genomic pattern.

In some embodiments, a plurality of nucleic acid molecules is extracted from the cell-free biological sample and subjected to sequencing to generate a plurality of sequencing reads. The nucleic acid molecules may comprise RNA or DNA. The nucleic acid molecules (e.g., RNA or DNA) may be extracted from the cell-free biological sample by a variety of methods, such as a nucleic acid extraction kits. The extraction method may extract all RNA or DNA molecules from a sample. Alternatively, the extract method may selectively extract a portion of RNA or DNA molecules from a sample. Extracted RNA molecules from a sample may be converted to DNA molecules by reverse transcription (RT).

Sequencing of nucleic acid molecules may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, sequencing-by-hybridization, and RNA-Seq (Illumina).

The sequencing may comprise nucleic acid amplification (e.g., of RNA or DNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., RNA or DNA) to a desired input quantity for subsequent sequencing. In some cases, the PCR may be used for global amplification of target nucleic acids. This amplification may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci associated with liver disease. The sequencing may comprise use of simultaneous RT and PCR, such as a OneStep RT-PCR kit protocol by Qiagen, NEB, Thermo Fisher Scientific, or Bio-Rad.

RNA or DNA molecules isolated or extracted from a cell-free biological sample may be tagged, e.g., with identifiable tags, to allow for multiplexing of a plurality of samples. Any number of RNA or DNA samples may be multiplexed. For example, a multiplexed reaction may contain RNA or DNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial cell-free biological samples. For example, a plurality of cell-free biological samples may be tagged with sample barcodes such that each DNA molecule may be traced back to the sample (and the subject) from which the DNA molecule originated. Such tags may be attached to RNA or DNA molecules by ligation or by PCR amplification with primers.

After subjecting the nucleic acid molecules to sequencing, suitable bioinformatics processes may be performed on the sequence reads to generate the data indicative of the presence, absence, or relative assessment of the liver disease. For example, the sequence reads may be aligned to one or more reference genomes (e.g., a genome of one or more species such as a human genome). The aligned sequence reads may be quantified at one or more genomic loci to generate the datasets indicative of the liver disease. For example, quantification of sequences corresponding to a plurality of genomic loci associated with liver disease may generate the datasets indicative of the liver disease.

In some cases, the cell-free biological sample may be processed without any nucleic acid extraction. For example, the liver disease may be identified or monitored in the subject by using probes configured to selectively enrich nucleic acid (e.g., RNA or DNA) molecules corresponding to the plurality of liver disease-associated genomic loci. The probes may be nucleic acid primers. The probes may have sequence complementarity with nucleic acid sequences from one or more of the plurality of liver disease-associated genomic loci or genomic regions. The plurality of liver disease-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, or more distinct liver disease-associated genomic loci or genomic regions. The plurality of liver disease-associated genomic loci or genomic regions may comprise one or more members (e.g., 1, 2, 3, 4, 5, 6, 7, 8, 9, 10, 11, 12, 13, 14, 15, 16, 17, 18, 19, 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, about 1000, or more) selected from the group consisting of genes listed in TABLE 1. The liver disease-associated genomic loci or genomic regions may be associated with age, race, ethnicity, BMI, blood glucose levels, or other liver disease states or complications.

TABLE 1

| | | | | | |
|---|---|---|---|---|---|
| A2M | A2ML1 | AACSP1 | AADACL3 | AADACL4 | AAMDC |
| AARD | AATK | ABAT | ABBA01000935.2 | ABCA11P | ABCA17P |
| ABCA7 | ABCB11 | ABCC2 | ABCC5 | ABCC6P1 | ABHD12 |
| ABHD15-AS1 | ABHD3 | ABHD6 | ABR | AC000124.1 | AC002059.3 |
| AC002310.2 | AC002985.1 | AC003043.2 | AC003950.1 | AC003973.2 | AC004009.1 |
| AC004052.1 | AC004080.6 | AC004147.4 | AC004156.1 | AC004231.1 | AC004522.4 |
| AC004528.1 | AC004593.2 | AC004594.1 | AC004637.1 | AC004672.1 | AC004687.2 |
| AC004702.1 | AC004784.1 | AC004828.2 | AC004834.1 | AC004917.1 | AC004922.1 |
| AC004943.2 | AC004951.4 | AC004980.1 | AC004987.2 | AC005020.2 | AC005050.2 |
| AC005064.1 | AC005144.1 | AC005225.2 | AC005229.5 | AC005258.1 | AC005264.1 |
| AC005280.1 | AC005324.4 | AC005387.2 | AC005476.2 | AC005520.1 | AC005520.5 |
| AC005599.1 | AC005622.1 | AC005670.2 | AC005670.3 | AC005697.1 | AC005702.1 |
| AC005726.1 | AC005726.3 | AC005786.3 | AC005796.1 | AC005833.1 | AC005837.2 |
| AC005943.1 | AC005962.1 | AC005972.3 | AC006030.1 | AC006059.2 | AC006064.6 |
| AC006130.1 | AC006355.2 | AC006369.1 | AC006372.3 | AC006449.6 | AC006453.1 |
| AC006455.1 | AC006455.4 | AC006486.1 | AC006487.1 | AC006511.6 | AC006525.1 |
| AC006581.2 | AC006972.1 | AC007161.3 | AC007192.2 | AC007216.3 | AC007216.4 |
| AC007319.1 | AC007333.2 | AC007344.1 | AC007349.1 | AC007368.1 | AC007375.2 |
| AC007389.1 | AC007389.2 | AC007461.1 | AC007608.1 | AC007663.2 | AC007666.2 |
| AC007879.1 | AC007879.3 | AC007906.2 | AC007922.3 | AC007998.2 | AC007998.3 |
| AC008014.1 | AC008035.1 | AC008050.1 | AC008083.2 | AC008105.2 | AC008459.1 |
| AC008467.1 | AC008507.5 | AC008537.1 | AC008554.1 | AC008567.2 | AC008568.1 |
| AC008635.2 | AC008667.1 | AC008676.3 | AC008687.1 | AC008691.1 | AC008695.1 |
| AC008758.1 | AC008758.5 | AC008758.6 | AC008763.2 | AC008764.1 | AC008802.1 |
| AC008825.1 | AC008878.3 | AC008945.2 | AC008957.1 | AC009054.1 | AC009063.3 |
| AC009065.2 | AC009065.7 | AC009070.1 | AC009093.10 | AC009093.11 | AC009117.2 |
| AC009133.1 | AC009133.6 | AC009142.1 | AC009163.3 | AC009226.1 | AC009242.1 |
| AC009264.1 | AC009292.1 | AC009292.2 | AC009320.1 | AC009396.1 | AC009396.3 |
| AC009403.1 | AC009403.2 | AC009412.1 | AC009522.1 | AC009554.1 | AC009554.2 |
| AC009597.1 | AC009879.2 | AC009879.3 | AC009879.4 | AC010133.1 | AC010197.1 |
| AC010197.2 | AC010247.1 | AC010273.2 | AC010319.2 | AC010320.2 | AC010327.4 |
| AC010327.5 | AC010336.1 | AC010422.2 | AC010422.6 | AC010442.3 | AC010519.1 |
| AC010533.1 | AC010616.1 | AC010634.1 | AC010754.1 | AC010894.2 | AC010998.1 |
| AC011092.2 | AC011287.1 | AC011290.1 | AC011294.1 | AC011330.3 | AC011369.1 |
| AC011444.1 | AC011447.3 | AC011448.1 | AC011462.1 | AC011468.2 | AC011472.2 |
| AC011472.3 | AC011476.2 | AC011477.6 | AC011479.1 | AC011498.7 | AC011500.2 |
| AC011509.2 | AC011611.2 | AC011676.5 | AC011718.1 | AC011747.1 | AC012063.1 |
| AC012081.1 | AC012146.1 | AC012158.1 | AC012213.5 | AC012309.1 | AC012322.1 |
| AC012358.3 | AC012363.1 | AC012405.1 | AC012414.2 | AC012414.6 | AC012459.1 |
| AC012485.1 | AC012560.1 | AC012618.3 | AC012651.1 | AC012668.2 | AC012668.3 |
| AC013402.3 | AC013717.1 | AC015712.4 | AC015712.5 | AC015712.6 | AC015845.2 |
| AC015878.1 | AC015908.2 | AC015908.7 | AC015911.11 | AC015923.1 | AC015971.1 |
| AC016573.1 | AC016582.1 | AC016582.2 | AC016587.1 | AC016590.1 | AC016590.4 |
| AC016821.1 | AC016866.2 | AC016885.1 | AC016907.2 | AC016987.1 | AC017002.2 |
| AC017002.3 | AC017083.3 | AC018618.1 | AC018630.2 | AC018644.1 | AC018653.1 |
| AC018680.1 | AC018730.1 | AC018731.1 | AC018809.3 | AC018816.1 | AC018865.2 |
| AC019131.1 | AC019131.3 | AC019197.1 | AC020663.2 | AC020663.4 | AC020687.1 |
| AC020904.2 | AC020908.3 | AC020910.6 | AC020911.2 | AC020916.1 | AC020917.2 |
| AC020922.1 | AC021055.1 | AC021078.1 | AC021086.1 | AC021087.5 | AC021092.1 |
| AC021127.1 | AC021231.1 | AC021351.1 | AC021393.1 | AC021573.1 | AC021660.3 |
| AC021683.4 | AC021683.5 | AC021733.4 | AC021979.1 | AC022034.3 | AC022098.4 |
| AC022126.1 | AC022382.2 | AC022414.1 | AC022558.2 | AC022726.2 | AC022915.2 |
| AC023034.1 | AC023055.1 | AC023421.2 | AC023469.1 | AC023490.4 | AC023509.1 |
| AC023855.1 | AC023905.1 | AC023906.5 | AC024236.1 | AC024267.4 | AC024270.2 |
| AC024382.1 | AC024558.2 | AC024597.1 | AC024598.1 | AC024610.2 | AC024933.2 |
| AC025165.3 | AC025279.1 | AC025283.2 | AC025287.1 | AC025682.1 | AC025917.1 |
| AC026369.3 | AC026412.1 | AC026495.1 | AC026495.2 | AC026583.1 | AC026746.1 |
| AC026992.2 | AC027045.2 | AC027290.2 | AC027514.1 | AC027601.2 | AC027601.5 |
| AC027613.1 | AC027688.1 | AC027801.2 | AC027808.1 | AC027808.2 | AC034102.2 |
| AC034102.3 | AC034102.4 | AC034111.2 | AC034154.1 | AC034195.1 | AC034228.3 |
| AC036214.3 | AC037486.1 | AC046129.1 | AC046134.2 | AC046143.1 | AC046168.1 |
| AC046168.2 | AC051619.3 | AC053513.1 | AC058822.1 | AC060809.1 | AC060814.4 |
| AC061979.1 | AC063952.1 | AC066613.2 | AC066615.1 | AC067751.1 | AC067752.1 |
| AC067930.4 | AC067956.1 | AC067968.1 | AC068051.1 | AC068205.1 | AC068205.2 |
| AC068282.1 | AC068308.1 | AC068418.1 | AC068446.2 | AC068446.3 | AC068473.1 |
| AC068633.1 | AC068722.1 | AC068724.5 | AC068733.3 | AC068987.2 | AC068987.3 |
| AC069152.1 | AC069234.1 | AC069281.2 | AC069287.3 | AC069288.1 | AC069368.1 |
| AC069368.2 | AC069410.1 | AC073107.1 | AC073114.1 | AC073176.2 | AC073283.3 |
| AC073320.1 | AC073475.1 | AC073612.1 | AC073842.2 | AC073863.1 | AC073941.1 |
| AC073957.1 | AC073957.3 | AC074032.1 | AC074051.4 | AC074091.2 | AC074139.1 |
| AC074143.1 | AC074286.1 | AC077690.1 | AC078815.1 | AC078881.1 | AC078905.1 |
| AC078923.1 | AC078927.1 | AC078980.1 | AC079035.1 | AC079142.1 | AC079145.1 |
| AC079160.1 | AC079313.1 | AC079313.2 | AC079414.1 | AC079760.1 | AC079760.2 |
| AC079790.2 | AC079793.1 | AC079848.1 | AC079848.2 | AC079921.1 | AC079988.1 |
| AC080079.2 | AC080162.1 | AC083798.2 | AC083841.1 | AC083841.2 | AC083841.4 |
| AC083973.1 | AC084756.2 | AC084834.1 | AC087164.1 | AC087241.2 | AC087276.2 |
| AC087289.3 | AC087294.1 | AC087482.1 | AC087521.3 | AC087564.1 | AC087633.2 |
| AC087721.2 | AC090061.1 | AC090115.1 | AC090192.2 | AC090193.1 | AC090282.1 |
| AC090527.3 | AC090559.1 | AC090578.1 | AC090589.2 | AC090617.5 | AC090644.1 |
| AC090888.3 | AC090907.2 | AC090912.1 | AC090912.2 | AC090983.2 | AC090993.1 |
| AC091078.1 | AC091096.1 | AC091132.4 | AC091132.5 | AC091151.7 | AC091153.2 |
| AC091178.2 | AC091212.1 | AC092070.2 | AC092100.1 | AC092117.2 | AC092119.1 |
| AC092131.1 | AC092155.1 | AC092164.1 | AC092316.1 | AC092329.3 | AC092353.1 |
| AC092353.2 | AC092375.2 | AC092445.1 | AC092535.4 | AC092567.1 | AC092574.2 |
| AC092640.1 | AC092647.5 | AC092650.1 | AC092691.1 | AC092718.8 | AC092745.2 |
| AC092802.1 | AC092803.2 | AC092813.1 | AC092821.3 | AC092845.1 | AC092865.3 |
| AC092910.3 | AC092957.1 | AC092958.4 | AC092966.1 | AC093001.1 | AC093110.1 |
| AC093151.2 | AC093151.7 | AC093155.3 | AC093227.2 | AC093274.1 | AC093323.3 |
| AC093423.2 | AC093423.3 | AC093459.1 | AC093523.1 | AC093525.9 | AC093599.1 |
| AC093627.4 | AC093655.1 | AC093899.2 | AC096577.1 | AC096639.1 | AC096887.1 |
| AC097104.1 | AC097487.1 | AC097511.1 | AC097515.1 | AC097522.2 | AC097634.4 |
| AC097636.1 | AC098588.2 | AC098588.3 | AC098850.3 | AC099487.2 | AC099489.1 |
| AC099506.1 | AC099506.3 | AC099681.1 | AC099791.3 | AC099811.1 | AC099850.2 |
| AC100786.1 | AC100807.2 | AC103758.1 | AC103855.2 | AC103871.1 | AC104041.1 |
| AC104083.1 | AC104109.3 | AC104123.1 | AC104435.2 | AC104472.3 | AC104532.1 |
| AC104574.2 | AC104596.1 | AC104667.1 | AC104781.1 | AC105206.2 | AC105411.1 |
| AC105430.1 | AC105760.1 | AC105941.1 | AC106782.1 | AC106782.4 | AC106791.1 |
| AC106795.1 | AC106795.3 | AC106799.2 | AC106886.6 | AC107032.2 | AC107032.3 |
| AC107223.1 | AC107918.4 | AC108488.2 | AC108488.3 | AC108734.4 | AC108941.2 |
| AC109460.2 | AC111182.1 | AC112198.2 | AC112206.2 | AC113391.1 | AC113391.2 |
| AC113618.2 | AC114271.1 | AC114311.1 | AC114781.2 | AC114930.1 | AC114947.1 |
| AC114956.2 | AC114971.1 | AC114977.1 | AC114982.3 | AC115220.1 | AC115622.1 |
| AC116025.1 | AC116337.3 | AC116362.1 | AC116366.2 | AC116903.2 | AC117834.2 |
| AC119150.1 | AC119674.1 | AC119674.2 | AC120036.1 | AC120114.4 | AC122685.1 |
| AC123912.4 | AC124017.1 | AC124319.2 | AC126182.3 | AC126283.2 | AC126335.2 |
| AC126603.1 | AC126755.6 | AC127502.1 | AC127526.3 | AC128685.1 | AC131009.2 |
| AC131160.1 | AC131274.1 | AC131274.2 | AC131274.3 | AC131571.1 | AC132825.1 |
| AC133555.6 | AC133644.2 | AC134980.1 | AC134980.2 | AC134980.3 | AC135050.3 |
| AC135166.1 | AC135586.2 | AC135731.2 | AC135983.5 | AC136628.4 | AC137494.1 |
| AC137579.1 | AC137579.2 | AC137735.1 | AC137735.2 | AC137800.1 | AC137894.1 |
| AC138123.1 | AC138207.6 | AC138207.7 | AC138207.8 | AC138305.1 | AC138409.2 |
| AC138627.1 | AC138696.1 | AC138761.1 | AC138811.1 | AC138819.1 | AC138866.2 |
| AC138894.1 | AC138904.3 | AC138907.8 | AC138907.9 | AC138932.1 | AC138965.2 |
| AC139530.2 | AC139769.1 | AC139769.2 | AC139887.2 | AC140479.3 | AC140479.4 |
| AC140504.1 | AC141586.1 | AC144573.1 | AC145285.4 | AC148477.3 | AC211433.1 |
| AC211476.3 | AC211476.8 | AC231533.1 | AC233699.1 | AC234582.1 | AC234782.4 |
| AC241952.1 | AC243547.3 | AC243571.2 | AC243772.3 | AC243829.2 | AC243964.3 |
| AC244517.1 | AC244517.2 | AC245128.1 | AC245297.1 | AC245748.2 | AC253536.1 |
| ACACB | ACAD10 | ACBD6 | ACLY | ACMSD | ACOT1 |
| ACOX | ACOX2 | ACOXL | ACP1 | ACSBG2 | ACSL5 |
| ACTN1 | ACTR10 | ACTR3C | ACVR1C | ACYP2 | AD000090.1 |
| ADA2 | ADAL | ADAM12 | ADAM17 | ADAM19 | ADAM2 |
| ADAM24P | ADAMTS10 | ADAMTS2 | ADAMTS20 | ADAMTS3 | ADAMTS4 |
| ADAMTS7P4 | ADAMTSL5 | ADAP1 | ADAP2 | ADAR | ADARB1 |
| ADARB2 | ADAT2 | ADCY1 | ADCY2 | ADCY9 | ADD1 |
| ADD3-AS1 | ADGRB1 | ADGRB3 | ADGRD1 | ADGRD2 | ADGRF2 |
| ADGRF3 | ADHFE1 | ADIPOR2 | ADK | ADORA1 | ADORA2A |
| ADORA2A-AS1 | ADORA2B | ADPRHL1 | ADRA1B | ADRB3 | ADRM1 |
| ADSS2 | ADTRP | AF064858.1 | AF064860.1 | AF117829.1 | AF241726.2 |
| AFG3L2 | AGAP1 | AGBL1 | AGBL4 | AGPAT1 | AGPAT5 |
| AGTPBP1 | AHCY | AHCYL2 | AHNAK2 | AHRR | AIG1 |
| AJ003147.2 | AJ009632.2 | AJ011931.2 | AK3 | AK3P2 | AK5 |
| AK7 | AK9 | AKAIN1 | AKAP1 | AKAP10 | AKAP9 |
| AKNAD1 | AKR1B10 | AKR1C3 | AKR1E2 | AKR7L | AL008633.1 |
| AL008727.1 | AL008730.1 | AL022311.1 | AL023495.1 | AL023755.1 | AL023882.1 |
| AL024498.2 | AL024508.1 | AL031008.1 | AL031123.1 | AL031282.2 | AL031601.2 |
| AL031602.2 | AL031708.1 | AL031710.1 | AL033504.1 | AL033523.1 | AL035071.2 |
| AL035401.1 | AL035443.1 | AL035446.2 | AL035458.2 | AL035461.3 | AL035653.1 |
| AL049651.1 | AL049777.1 | AL049828.1 | AL049828.2 | AL049870.2 | AL096854.1 |
| AL109824.1 | AL109829.1 | AL110114.1 | AL110292.1 | AL117190.1 | AL117190.2 |
| AL117329.1 | AL117372.1 | AL118558.1 | AL121821.2 | AL121900.1 | AL121910.1 |
| AL121974.1 | AL122035.1 | AL132671.2 | AL132857.1 | AL133297.1 | AL133297.2 |
| AL133318.1 | AL133342.1 | AL133372.2 | AL133410.1 | AL133467.3 | AL133481.1 |
| AL133492.1 | AL133538.1 | AL135878.1 | AL135926.1 | AL136115.2 | AL136119.1 |
| AL136171.2 | AL136456.1 | AL136981.1 | AL136985.1 | AL136985.3 | AL136988.2 |
| AL137003.1 | AL137058.1 | AL137139.2 | AL137157.1 | AL137191.1 | AL138720.1 |
| AL138752.2 | AL138930.1 | AL139246.5 | AL139327.2 | AL139383.1 | AL139423.2 |
| AL139807.1 | AL139815.1 | AL157371.2 | AL157388.1 | AL157392.5 | AL157414.1 |
| AL157778.1 | AL157886.1 | AL157911.1 | AL158011.1 | AL158066.1 | AL158195.1 |
| AL158198.1 | AL158198.2 | AL159163.1 | AL160272.2 | AL160396.1 | AL161716.1 |
| AL161725.1 | AL161912.4 | AL161941.1 | AL162253.2 | AL162425.1 | AL162458.1 |
| AL162464.1 | AL162464.2 | AL162717.1 | AL162724.1 | AL162725.2 | AL162726.3 |
| AL162727.2 | AL162872.1 | AL163952.1 | AL353052.1 | AL353588.1 | AL353604.1 |
| AL353626.1 | AL353660.1 | AL353697.1 | AL353743.1 | AL354754.1 | AL354833.1 |
| AL354863.1 | AL354994.1 | AL355073.1 | AL355102.4 | AL355103.1 | AL355499.1 |
| AL355499.2 | AL355836.3 | AL355881.1 | AL356218.2 | AL356234.2 | AL356295.1 |
| AL357143.1 | AL357375.1 | AL357507.1 | AL357793.1 | AL358292.1 | AL358394.2 |
| AL359076.1 | AL359313.1 | AL359317.2 | AL359636.2 | AL359649.1 | AL359710.1 |
| AL359736.1 | AL359854.1 | AL359915.1 | AL359922.1 | AL365181.3 | AL365256.1 |
| AL365272.1 | AL390334.1 | AL390728.5 | AL390800.1 | AL390860.1 | AL391422.2 |
| AL391811.1 | AL392003.2 | AL392083.1 | AL392185.1 | AL445250.1 | AL445423.3 |
| AL445433.2 | AL445584.2 | AL445928.2 | AL450322.2 | AL450352.1 | AL450992.1 |
| AL451062.3 | AL451164.2 | AL512324.3 | AL512328.1 | AL512356.1 | AL512634.1 |
| AL513128.1 | AL513412.1 | AL583836.1 | AL589666.1 | AL589693.1 | AL589740.1 |
| AL589923.1 | AL590652.1 | AL590666.2 | AL590807.1 | AL590867.1 | AL591441.1 |
| AL591518.1 | AL592295.3 | AL592402.1 | AL592429.2 | AL603840.1 | AL606534.1 |
| AL606753.2 | AL606804.1 | AL606970.3 | AL607033.1 | AL645922.1 | AL662884.2 |
| AL669831.1 | AL671762.1 | AL691403.1 | AL691420.1 | AL731702.1 | AL732314.4 |
| AL732314.6 | AL732406.1 | AL773573.1 | AL845472.2 | ALB | ALDH |
| ALDH1A2 | ALDH1L1 | ALDH3A2 | ALDH5A1 | ALDH7A1 | ALDOC |
| ALKBH2 | ALKBH8 | ALMS1 | ALOX15P1 | ALPG | ALPL |
| AMBRA1 | AMELY | AMN1 | AMOTL1 | AMPD3 | AMPH |
| AMZ2 | ANAPC1 | ANAPC5 | ANGEL1 | ANGPT1 | ANGPTL6 |
| ANK1 | ANKFN1 | ANKFY1 | ANKLE2 | ANKMY1 | ANKRD12 |
| ANKRD13C | ANKRD20A1 | ANKRD20A21P | ANKRD20A7P | ANKRD20A9P | ANKRD24 |
| ANKRD26 | ANKRD28 | ANKRD31 | ANKRD33B | ANKRD36 | ANKRD36B |
| ANKRD36C | ANKRD44 | ANKRD54 | ANKRD55 | ANKRD61 | ANKS6 |
| ANO1 | ANO10 | ANO7 | ANP32A | ANTXR1 | ANTXR2 |
| ANXA4 | ANXA6 | AOX2P | AOX3P | AP000282.1 | AP000311.1 |
| AP000317.1 | AP000317.2 | AP000331.1 | AP000350.3 | AP000442.1 | AP000688.1 |
| AP000753.1 | AP000769.1 | AP000919.2 | AP000944.5 | AP001011.1 | AP001021.3 |
| AP001037.1 | AP001062.1 | AP001107.9 | AP001109.1 | AP001160.3 | AP001189.4 |
| AP001267.5 | AP001273.2 | AP001605.1 | AP001830.2 | AP001924.1 | AP001931.1 |
| AP001931.2 | AP001977.1 | AP002336.2 | AP002373.1 | AP002381.2 | AP002439.1 |
| AP002518.2 | AP002748.5 | AP002761.2 | AP002812.2 | AP002884.2 | AP003108.2 |
| AP003393.1 | AP003680.1 | AP003717.1 | AP005230.1 | AP005263.1 | AP005329.2 |
| AP005717.2 | AP006545.3 | AP006621.1 | AP1M1 | AP2A2 | AP2B1 |
| AP2S1 | AP3M2 | AP3S2 | APBA1 | APBB2 | APC2 |
| API5 | APMAP | APOBEC3A | APOBEC3B | APOC4 | APOH |
| APOLD1 | APPL2 | AQP4-AS1 | AQP9 | ARAP2 | ARF4 |
| ARFGEF3 | ARFIP1 | ARG1 | ARHGAP10 | ARHGAP12 | ARHGAP15 |
| ARHGAP17 | ARHGAP18 | ARHGAP19 | ARHGAP19- | ARHGAP21 | ARHGAP23 |
| | | | SLIT1 | | |
| ARHGAP24 | ARHGAP26 | ARHGAP45 | ARHGDIA | ARHGEF10L | ARHGEF18 |
| ARHGEF26-AS1 | ARHGEF3 | ARHGEF4 | ARHGEF9 | ARID1A | ARID1B |
| ARID2 | ARID3A | ARID5B | ARL15 | ARL17B | ARL6 |
| ARLNC1 | ARMC4 | ARMC4P1 | ARMC7 | ARMC8 | ARMC9 |
| ARMH4 | ARNT | ARNTL | ARPC1A | ARPC4 | ARPC4-TTLL3 |
| ARPIN-AP3S2 | ARRDC2 | ARSB | ARSF | ARVCF | ASAP3 |
| ASB13 | ASCC1 | ASIC1 | ASIC2 | ASPA | ASTN1 |
| ASTN2 | ASTN2-AS1 | ASXL2 | ATAD2B | ATAD3A | ATF3 |
| ATF6 | ATF6B | ATF7IP2 | ATG13 | ATG14 | ATG16L1 |
| ATG5 | ATG7 | ATIC | ATL2 | ATP10A | ATP13A1 |
| ATP13A4 | ATP13A5 | ATP1A3 | ATP2A1 | ATP2B2 | ATP5MC2 |
| ATP5MF-PTCD1 | ATP5PD | ATP6V0E2 | ATP6V1D | ATP6V1H | ATP8A1 |
| ATP8A2P3 | ATP9B | ATRIP | ATRX | ATXN1 | ATXN10 |
| ATXN2 | ATXN7 | ATXN7L3 | AUP1 | AUTS2 | AVEN |
| AXDND1 | AXIN1 | AZGP1 | AZIN2 | B3GAT3 | B3GNT3 |
| B4GALT1 | B4GALT5 | B4GALT6 | BABAM2 | BACE1 | BACE1-AS |
| BAHCC1 | BAIAP2 | BAIAP2L1 | BAIAP2L2 | BANK1 | BASP1 |
| BATF | BAX | BAZ1B | BAZ2B | BBS1 | BCAN |
| BCAR3 | BCAS2P2 | BCAS3 | BCKDHA | BCL11A | BCL2 |
| BCL2L1 | BCL2L13 | BCL7A | BCL7B | BCL7C | BCL9 |
| BCL9L | BCO1 | BCR | BCRP2 | BDKRB1 | BEND3 |
| BEST1 | BET1L | BFSP1 | BHLHE40-AS1 | BICC1 | BICDL1 |
| BICRA | BICRAL | BIN1 | BIN2 | BIRC2 | BLM |
| BMERB1 | BMP7 | BMPR1B | BMS1P15 | BNC2 | BNIP3 |
| BORCS5 | BORCS8-MEF2B | BPTF | BRAP | BRD1 | BRD4 |
| BRD9 | BRF1 | BRI3 | BRINP1 | BRIP1 | BRMS1 |
| BRSK1 | BRSK2 | BRWD1 | BSDC1 | BTBD11 | BTBD2 |
| BTBD8 | BTBD9 | BTD | BTF3P8 | BTN3A2 | BTRC |
| BX255923.1 | BX664718.2 | BZW1-AS1 | BZW2 | C10orf71 | C10orf95 |
| C11orf49 | C11orf58 | C11orf65 | C11orf80 | C11orf94 | C12orf40 |
| C12orf65 | C12orf75 | C13orf46 | C14orf39 | C15orf41 | C17orf100 |
| C17orf67 | C17orf78 | C18orf21 | C18orf32 | C19orf38 | C19orf44 |
| C1D | Clorf109 | Clorf127 | Clorf141 | Clorf21 | Clorf61 |
| C1QTNF6 | C1QTNF7-AS1 | C1S | C2 | C21orf62-AS1 | C22orf24 |
| C22orf31 | C22orf34 | C22orf39 | C2CD3 | C2orf42 | C2orf50 |
| C2orf69 | C2orf88 | C3orf33 | C3orf49 | C3P1 | C4A |
| C4A-AS1 | C4BPA | C4orf17 | C5AR1 | C5orf15 | C5orf34 |
| C5orf46 | C5orf64 | C5orf66 | C6orf99 | C7orf33 | C7orf50 |
| C8orf31 | C8orf37-AS1 | C8orf44 | C8orf44-SGK3 | C8orf49 | C8orf74 |
| C9orf135 | C9orf43 | C9orf92 | CA15P1 | CA6 | CA8 |
| CAAP1 | CAB39 | CABIN1 | CABLES1 | CACHD1 | CACNA1A |
| CACNA1C | CACNA1E | CACNA1H | CACNA1I | CACNA2D3 | CACNG3 |
| CACUL1 | CADM1 | CADPS2 | CAGE1 | CALCB | CALCRL |
| CALML3-AS1 | CALML6 | CALN1 | CAMK1D | CAMK2B | CAMK2G |
| CAMK4 | CAMKMT | CAMSAP3 | CAMTA1 | CANX | CAP1 |
| CAP2 | CAPG | CAPN1 | CAPN15 | CAPN3 | CAPN7 |
| CAPN9 | CAPRIN1 | CAPZA1 | CAPZB | CARD18 | CARF |
| CARM1P1 | CASC11 | CASC15 | CASC16 | CASC2 | CASC8 |
| CASK | CASP1 | CASP8 | CASP9 | CASS4 | CAST |
| CASTOR2 | CATIP | CAVIN1 | CBX2 | CBX7 | CBY1 |
| CBY1P1 | CC2D2B | CCDC125 | CCDC127 | CCDC13 | CCDC141 |
| CCDC144A | CCDC148 | CCDC148-AS1 | CCDC150 | CCDC154 | CCDC162P |
| CCDC167 | CCDC171 | CCDC173 | CCDC180 | CCDC18-AS1 | CCDC190 |
| CCDC22 | CCDC26 | CCDC27 | CCDC3 | CCDC30 | CCDC33 |
| CCDC39 | CCDC40 | CCDC57 | CCDC63 | CCDC66 | CCDC7 |
| CCDC70 | CCDC81 | CCDC87 | CCDC88A | CCDC91 | CCL5 |
| CCNB2 | CCNB3 | CCND3 | CCNDBP1 | CCNT2 | CCNT2-AS1 |
| CCNY | CCNYL1 | CCR6 | CCR7 | CCSAP | CCSER1 |
| CCT6B | CCT7 | CD19 | CD226 | CD247 | CD300LF |
| CD38 | CD3E | CD4 | CD58 | CD6 | CD69 |
| CD72 | CD80 | CD81-AS1 | CD83 | CD84 | CD8B |
| CD96 | CD99 | CD99P1 | CDC123 | CDC14A | CDC16 |
| CDC20B | CDC25A | CDC25C | CDC37L1 | CDC40 | CDC42 |
| CDC42BPA | CDC42SE2 | CDCA3 | CDCA7 | CDH1 | CDH11 |
| CDH13 | CDH17 | CDH23 | CDH3 | CDH4 | CDH5 |
| CDH8 | CDHR2 | CDHR3 | CDK11A | CDK11B | CDK12 |
| CDK13 | CDK14 | CDK15 | CDK2AP1 | CDK5RAP2 | CDK8 |
| CDKAL1 | CDKL2 | CDKN2AIPNL | CDKN2B-AS1 | CDR2 | CDS2 |
| CDX1 | CDYL | CDYL2 | CEACAM22P | CEACAM7 | CEBPG |
| CECR2 | CELF5 | CELSR1 | CELSR3 | CEMIP | CEMIP2 |
| CENPH | CENPM | CENPX | CEP112 | CEP128 | CEP131 |
| CEP164 | CEP164P1 | CEP170P1 | CEP20 | CEP295NL | CEP350 |
| CEP57 | CEP70 | CEP72 | CEP76 | CERS4 | CERS5 |
| CES4A | CFAP161 | CFAP20DC | CFAP20DC-AS1 | CFAP251 | CFAP410 |
| CFAP52 | CFAP57 | CFAP65 | CFAP74 | CFAP97D2 | CFDP1 |
| CFI | CFL1 | CFP | CGNL1 | CHCHD6 | CHERP |
| CHFR | CHGB | CHID1 | CHMP3 | CHMP6 | CHN2 |
| CHODL | CHRFAM7A | CHRM2 | CHRM5 | CHRNA10 | CHRNA6 |
| CHST12 | CHST8 | CHTF18 | CIAO2A | CIDEA | CIPC |
| CIRBP | CIT | CKAP5 | CKM | CKMT1B | CLASP2 |
| CLCA4 | CLCA4-AS1 | CLCC1 | CLDN4 | CLDND1 | CLDND2 |
| CLEC10A | CLEC16A | CLEC2D | CLEC2L | CLEC3A | CLEC6A |
| CLHC1 | CLIC2 | CLIC4 | CLIP1 | CLIP2 | CLMN |
| CLN3 | CLN8 | CLNS1A | CLPP | CLPTM1 | CLSTN2 |
| CLTA | CLTB | CLTC | CLTCL1 | CLVS1 | CLYBL |
| CMBL | CMC1 | CMIP | CMTM8 | CNGA1 | CNGA3 |
| CNIH3 | CNIH3-AS2 | CNN2 | CNNM1 | CNOT10 | CNOT2 |
| CNOT6L | CNPY1 | CNPY4 | CNTLN | CNTNAP2 | CNTNAP3 |
| CNTNAP3B | CNTNAP3P5 | CNTNAP5 | CNTRL | COA7 | COIL |
| COL13A1 | COL1A1 | COL1A2 | COL22A1 | COL23A1 | COL24A1 |
| COL25A1 | COL26A1 | COL27A1 | COL28A1 | COL4A1 | COL4A2 |
| COL4A2-AS1 | COL5A2 | COL5A3 | COL6A4P1 | COL8A2 | COLEC12 |
| COMMD7 | COMP | COPB2 | COPZ2 | COQ5 | CORO1B |
| CORO1C | CORO2B | CORO7 | CORO7-PAM16 | COX19 | COX6B1 |
| COX7A2L | CPAMD8 | CPEB3 | CPLANE1 | CPLX2 | CPN1 |
| CPNE4 | CPNE5 | CPPED1 | CPQ | CPSF3 | CPSF4 |
| CPT1A | CPXM1 | CPXM2 | CR1L | CR381670.1 | CR381670.2 |
| CR382285.1 | CRACD | CRACDL | CRACR2A | CRACR2B | CRADD |
| CRAMP1 | CRAT37 | CRCT1 | CREB3L2 | CREBBP | CREG2 |
| CRELD1 | CRKL | CRLF1 | CROCC | CROCC2 | CROCCP3 |
| CRPPA | CRTC1 | CRTC3-AS1 | CRYL1 | CSF1R | CSF2 |
| CSF2RB | CSF3R | CSGALNACT1 | CSMD3 | CSNK1A1 | CSNK1D |
| CSNK1E | CSNK1G2 | CSNK2A1 | CSRP3 | CSTF3 | CT75 |
| CTBP1-DT | CTC1 | CTDSPL | CTDSPL2 | CTGF | CTH |
| CTIF | CTNNA1 | CTNNA1P1 | CTNNA3 | CTNND1 | CTR9 |
| CTSH | CTSS | CUBN | CUL1 | CUL4B | CUL7 |
| CUX1 | CWC27 | CXADR | CXCR2P1 | CXXC4-AS1 | CYB561A3 |
| CYB5A | CYB5D2 | CYBRD1 | CYFIP2 | CYP11B1 | CYP11B2 |
| CYP1B1-AS1 | CYP27A1 | CYP2C19 | CYP2D6 | CYP2D7 | CYP2F2P |
| CYP2G1P | CYP3A5 | CYP4F9P | CYRIA | CYTH4 | DAB2IP |
| DACT2 | DAG1 | DAPK1 | DAPK2 | DAPP1 | DAZAP1 |
| DAZL | DBF4B | DBT | DCAF10 | DCAF12 | DCAF17 |
| DCAF6 | DCAKD | DCBLD1 | DCC | DCDC1 | DCHS2 |
| DCLK2 | DCLRE1C | DCPS | DCTN1 | DCTN2 | DCUN1D1 |
| DCUN1D2 | DCUN1D4 | DCUN1D5 | DDC | DDI2 | DDIAS |
| DDX10 | DDX12P | DDX18P5 | DDX49 | DDX58 | DDX6 |
| DEFB1 | DEFB108A | DEFB114 | DEFB130A | DEFB134 | DELEC1 |
| DENND1A | DENND1C | DENND2B | DENND2C | DENND3 | DENND4C |
| DENND5A | DENND5B | DEPDC1-AS1 | DEPDC5 | DEPDC7 | DESI1 |
| DGCR5 | DGCR8 | DGKA | DGKH | DGKI | DGKQ |
| DGLUCY | DHRS7C | DHRS9 | DHRSX | DHX15 | DHX33 |
| DHX37 | DHX40 | DIAPH1 | DICER1 | DIP2C | DIPK1A |
| DIRC3 | DIS3L2 | DISC1 | DISC1-IT1 | DKK3 | DLAT |
| DLEC1 | DLEU1 | DLEU2 | DLEU2L | DLEU7 | DLG4 |
| DLG5 | DLGAP1 | DLGAP2 | DLGAP2-AS1 | DLGAP4 | DLX4 |
| DLX6-AS1 | DMGDH | DMRT1 | DMRTC1B | DMXL1 | DMXL2 |
| DNAAF5 | DNAH1 | DNAH10 | DNAH11 | DNAH12 | DNAH14 |
| DNAH2 | DNAH8 | DNAJB13 | DNAJB4 | DNAJC24 | DNAJC5B |
| DNAJC9 | DNASE1 | DNHD1 | DNM1L | DNM2 | DNM3 |
| DNMT3A | DNMT3B | DNPH1 | DNTTIP1 | DOC2B | DOCK1 |
| DOCK2 | DOCK3 | DOCK6 | DOCK7 | DOK5 | DOK7 |
| DPF3 | DPP3 | DPP6 | DPP9 | DPP9-AS1 | DPY30 |
| DPYD | DPYSL4 | DRAIC | DRAM2 | DRC1 | DRD4 |
| DSCAM | DSCAML1 | DSCC1 | DSCR4 | DSCR9 | DSG1-AS1 |
| DSG4 | DST | DSTN | DTD1 | DTD1-AS1 | DTHD1 |
| DTNB | DTNBP1 | DTWD2 | DTX4 | DUS1L | DUSP14 |
| DUSP16 | DUSP18 | DUSP7 | DUSP9 | DYM | DYNC1H1 |
| DYRK1A | DYSF | DZANK1 | E2F3 | EBNA1BP2 | ECE1 |
| ECE2 | ECEL1P1 | EDA | EDC3 | EDEM1 | EDIL3 |
| EDN1 | EDNRA | EDNRB | EEA1 | EEF1AKMT1 | EEF1AKMT3 |
| EEF1AKMT4- | EEF2 | EEPD1 | EFCAB2 | EFCAB5 | EFCAB7 |
| ECE2 | | | | | |
| EFCAB8 | EFHC1 | EFL1 | EFNB3 | EFR3A | EGFR-AS1 |
| EHBP1 | EHD4 | EID3 | EIF1 | EIF2A | EIF2AK1 |
| EIF2B3 | EIF2B5 | EIF3C | EIF4A3 | EIF4A3P1 | EIF4E |
| EIF4E1B | EIF4EBP2 | EIF4G3 | EIF6 | EIPR1 | ELAPOR1 |
| ELAPOR2 | ELAVL1 | ELDR | ELFN2 | ELL | ELMO1 |
| ELP4 | EMC3 | EML1 | EML6 | ENDOV | ENOX1 |
| ENPP2 | ENPP7P6 | ENTHD1 | ENTPD1-AS1 | ENTPD6 | EP300 |
| EP400 | EP400P1 | EPB41 | EPB41L1 | EPB41L4A | EPB41L4B |
| EPC1 | EPDR1 | EPHX2 | EPS15L1 | EPS8 | EPX |
| EPYC | ERBB3 | ERBIN | ERC1 | ERCC2 | ERCC3 |
| ERCC6 | ERCC6L2 | ERG | ERICH6B | ERLIN2 | ERO1B |
| ERP44 | ERVK13-1 | ERVK-28 | ESR1 | ESR2 | ESYT1 |
| ETF1 | ETV3L | ETV5 | ETV6 | ETV7 | EVI5 |
| EXD2 | EXOC3 | EXOC3L1 | EXOC4 | EXOC6 | EXOSC10 |
| EXTL3 | EYA3 | EYA4 | EYS | EZR-AS1 | F11-AS1 |
| F5 | F8 | FAAH | FAAHP1 | FAAP20 | FADS1 |
| FADS2 | FAF1 | FAHD2A | FAIM2 | FAM102B | FAM104A |
| FAM107B | FAM110B | FAM117B | FAM118A | FAM120AOS | FAM126A |
| FAM131C | FAM13B | FAM149B1 | FAM153A | FAM153CP | FAM163A |
| FAM167A | FAM167A-AS1 | FAM168A | FAM169A | FAM172A | FAM174B |
| FAM178B | FAM186A | FAM189A1 | FAM193A | FAM197Y7 | FAM214A |
| FAM219A | FAM220A | FAM222B | FAM227A | FAM230E | FAM230F |
| FAM234A | FAM27C | FAM41C | FAM53A | FAM66D | FAM71E2 |
| FAM74A7 | FAM76B | FAM81A | FAM81B | FAM83A | FAM83C |
| FAM83F | FAM86B1 | FAM86FP | FAM86JP | FAM90A12P | FAM90A24P |
| FAM90A26 | FAM90A8P | FAM91A1 | FAN1 | FANCC | FANCL |
| FAR2P1 | FARS2 | FASN | FASTKD1 | FASTKD2 | FAT4 |
| FBF1 | FBL | FBLN5 | FBN2 | FBP2P1 | FBRSL1 |
| FBXL13 | FBXL17 | FBXL18 | FBXL5 | FBXL8 | FBXO11 |
| FBXO21 | FBXO25 | FBXO42 | FBXW12 | FBXW7 | FCGBP |
| FCHSD1 | FCMR | FCRL4 | FDCSP | FEN1 | FER |
| FER1L6 | FER1L6-AS2 | FERMT3 | FEZ1 | FEZ2 | FGD4 |
| FGD6 | FGF12 | FGF13 | FGF8 | FGFR2 | FGFR3 |
| FGFRL1 | FGGY | FGR | FHIT | FHL1 | FHL2 |
| FHL3 | FIG4 | FIGNL1 | FIGNL2 | FIP1L1 | FKBP8 |
| FKRP | FLG-AS1 | FLJ36000 | FLJ40194 | FLJ46284 | FLNB |
| FLVCR1 | FLYWCH2 | FMN1 | FMNL1 | FNBP1L | FNDC3A |
| FNIP1 | FO393400.1 | FO681491.1 | FOLR3 | FOXG1-AS1 | FOXK1 |
| FOXL2 | FOXN2 | FOXO3 | FOXP1 | FRAS1 | FRG1CP |
| FRMD4B | FRMD5 | FRMPD4 | FRY | FRYL | FSD1 |
| FSD2 | FSIP2-AS1 | FSTL1 | FSTL4 | FSTL5 | FTCD |
| FTCDNL1 | FTX | FUBP1 | FURIN | FUT9 | FZD3 |
| FZR1 | G6PC | GAB2 | GABPB2 | GAD1 | GAL3ST1 |
| GALK2 | GALNT14 | GALNT16 | GALNT17 | GALNT2 | GALNT9 |
| GAN | GANAB | GANC | GAPDHP28 | GAPVD1 | GARS1 |
| GAS2 | GAS6-AS1 | GATA4 | GATM | GCFC2 | GCKR |
| GCLM | GCNT2 | GCSAML | GDI1 | GDPD4 | GEMIN6 |
| GET4 | GFRA2 | GGA1 | GGA3 | GGNBP1 | GGNBP2 |
| GIGYF2 | GIMD1 | GIPC2 | GIPR | GIT1 | GLB1 |
| GLCCI1-DT | GLDC | GLIPR1L2 | GLIS1 | GLIS3 | GLMP |
| GLOD4 | GLT1D1 | GLT8D1 | GLT8D2 | GLUD1 | GLYR1 |
| GM2A | GMDS | GMDS-DT | GMEB1 | GMIP | GML |
| GMNC | GNA12 | GNA14 | GNA15 | GNAI1 | GNAI3 |
| GNAL | GNAQ | GNAZ | GNB1 | GNE | GNG2 |
| GNG4 | GNG7 | GOLGA1 | GOLGA2P5 | GOLGA3 | GOLGA4 |
| GOLGA6A | GOLGA6L3 | GOLGA8H | GOLGB1 | GOLPH3 | GON4L |
| GORAB-AS1 | GORASP2 | GOSR2 | GOT1 | GPAT2P1 | GPATCH1 |
| GPATCH8 | GPC6 | GPHN | GPN1 | GPN3 | GPR137 |
| GPR137B | GPR141 | GPR146 | GPR149 | GPR179 | GPR35 |
| GPRC5B | GPRIN1 | GPSM2 | GRAMD1B | GRAP2 | GRB2 |
| GREB1 | GREB1L | GRHPR | GRIA2 | GRIA4 | GRID1 |
| GRID1-AS1 | GRID2IP | GRIK4 | GRIK5 | GRIN3A | GRIN3B |
| GRIP2 | GRK5 | GRM1 | GRM3 | GRM7 | GRM8 |
| GRPR | GS1-24F4.2 | GSDME | GSG1L | GSK3B | GSPT1 |
| GSS | GSTA5 | GTDC1 | GTF2B | GTF2F1 | GTF2F2 |
| GTF2H2 | GTF2I | GTF2IP8 | GTF2IRD1 | GTF2IRD2 | GTF3C1 |
| GTPBP2 | GTPBP4 | GTSE1 | GUCA1B | GUCY1A1 | GUCY2D |
| GUSBP16 | GUSBP3 | GXYLT2 | GYG2 | GYPC | GYS1 |
| GYS2 | H1-9P | H2AZ2P1 | HAGH | HAL | HAP1 |
| HARBI1 | HAS2-AS1 | HAS3 | HAUS5 | HAUS8 | HBZ |
| HCG20 | HCLS1 | HCRTR2 | HDAC1 | HDAC4 | HDAC5 |
| HDGF | HDGFL2 | HDHD5 | HDLBP | HEATR4 | HEATR5B |
| HECTD2 | HECTD3 | HECTD4 | HECW1 | HECW2 | HEG1 |
| HELZ | HEPHL1 | HERC2P3 | HERC2P4 | HERC4 | HGSNAT |
| HHAT | HHIPL2 | HHLA3 | HIBCH | HIC2 | HIP1 |
| HIPK2 | HIRA | HIVEP1 | HIVEP3 | HK3 | HLA-DQB2 |
| HLA-DRB6 | HLCS | HLCS-IT1 | HLX-AS1 | HMGB1 | HMGB3P22 |
| HMGXB3 | HNF1A | HNF1B | HNRNPDLP2 | HNRNPKP3 | HNRNPL |
| HNRNPM | HNRNPUL1 | HOOK1 | HOOK2 | HORMAD1 | HORMAD2 |
| HORMAD2-AS1 | HOXA3 | HOXA-AS2 | HOXA-AS3 | HOXB-AS1 | HPCAL1 |
| HPS5 | HPSE2 | HPYR1 | HRH2 | HRH3 | HS1BP3 |
| HS2ST1 | HS3ST2 | HS3ST3B1 | HS6ST3 | HSBP1 | HSD17B6 |
| HSF2BP | HSF4 | HSF5 | HSPA14 | HSPA5 | HSPB11 |
| HSPBAP1 | HSPG2 | HTR3C | HTR4 | HULC | HUWE1 |
| HVCN1 | HYDIN2 | IAH1 | ICA1L | ICAM3 | IDI1 |
| IDI2 | IDI2-AS1 | IDNK | IFNLR1 | IFT140 | IFT20 |
| IFT46 | IFT52 | IFT74 | IFT88 | IGDCC3 | IGF2BP3 |
| IGFALS | IGFL4 | IGHM | IGHMBP2 | IGIP | IGLV10-54 |
| IGSF1 | IGSF10 | IGSF11 | IGSF21 | IGSF9B | IKBKB |
| IL10RB | IL17RA | IL17REL | IL19 | IL1R1 | IL1RAPL1 |
| IL1RAPL2 | IL21R | IL27RA | IL2RB | IL31RA | IL4R |
| IL7 | IL9R | IMMP2L | IMMT | IMPA2 | INCENP |
| INHCAP | INO80C | INPP4B | INPP5J | INSL6 | INSR |
| INSRR | INTS13 | INTS4 | INTS4P1 | INTS7 | INTS9 |
| INVS | IPO11 | IPO9 | IPO9-AS1 | IPPK | IQCH |
| IQCH-AS1 | IQCK | IQCM | IQGAP2 | IQGAP3 | IQSEC1 |
| IQSEC3 | IQUB | IRAG1 | IRAK1BP1 | IRAK2 | IRAK3 |
| IRF1-AS1 | IRX4 | IST1 | ITGA2B | ITGA5 | ITGA9 |
| ITGA9-AS1 | ITGAE | ITGAM | ITGB3BP | ITGB5 | ITGBL1 |
| ITIH2 | ITIH5 | ITK | ITPKC | ITPR1 | ITPR2 |
| ITPR3 | ITSN1 | ITSN2 | JADE3 | JAG2 | JAK1 |
| JAK2 | JAKMIP3 | JMJD8 | JPT1 | JPT2 | JRK |
| JSRP1 | KALRN | KANK1P1 | KANSL1 | KAT14 | KAT2A |
| KAT6A | KAT6B | KAT7 | KATNAL2 | KAZN | KAZN-AS1 |
| KBTBD11 | KBTBD11-OT1 | KBTBD2 | KCMF1 | KCNC1 | KCND3 |
| KCNH2 | KCNIP4 | KCNJ6 | KCNK13 | KCNK9 | KCNMA1 |
| KCNN1 | KCNN3 | KCNQ1 | KCNQ1OT1 | KCNQ3 | KCNQ5 |
| KCTD10 | KCTD14 | KCTD2 | KCTD5 | KCTD8 | KDM2A |
| KDM2B | KDM4C | KDM5A | KDM5B | KHDC4 | KHDRBS1 |
| KHK | KIAA0232 | KIAA0319L | KIAA0586 | KIAA0930 | KIAA1328 |
| KIAA1614 | KIAA1841 | KIAA1958 | KIAA2012 | KIAA2026 | KIDINS220 |
| KIF13A | KIF15 | KIF19 | KIF1A | KIF3B | KIF5A |
| KIF9-AS1 | KIN | KIR2DL1 | KIR2DL4 | KIR2DP1 | KIR3DL1 |
| KIRREL1 | KIRREL3 | KLC3 | KLC4 | KLF12 | KLF3 |
| KLF3-AS1 | KLF7 | KLHDC10 | KLHL11 | KLHL18 | KLHL22 |
| KLHL23 | KLHL26 | KLHL28 | KLHL29 | KLHL3 | KLHL38 |
| KLHL41 | KMT2A | KMT2C | KMT2D | KMT5A | KMT5B |
| KPNA1 | KRBA2 | KREMEN1 | KRI1 | KRT23 | KRT34 |
| KRT35 | KRT79 | KRT8P38 | KRTAP10-13P | KRTDAP | KSR1 |
| KSR2 | KTN1 | KYAT3 | L34079.1 | L3MBTL3 | L3MBTL4 |
| LAMA3 | LAMA4 | LAMA5 | LAMB1 | LAMC1 | LAMP1 |
| LAMTOR5-AS1 | LARP4B | LARS2 | LARS2-AS1 | LAT2 | LATS1 |
| LCOR | LCORL | LDAH | LDHAL6A | LDHB | LDHC |
| LDLRAD3 | LDLRAD4 | LEMD2 | LEMD3 | LENG8-AS1 | LETM1 |
| LGR4 | LGR6 | LHFPL2 | LHFPL3 | LHFPL3-AS1 | LHX1-DT |
| LHX6 | LIFR-AS1 | LILRB4 | LIMA1 | LIMCH1 | LIMK1 |
| LINC00200 | LINC00205 | LINC00229 | LINC00251 | LINC00265 | LINC00271 |
| LINC00293 | LINC00298 | LINC00299 | LINC00301 | LINC00314 | LINC00319 |
| LINC00378 | LINC00393 | LINC00411 | LINC00446 | LINC00457 | LINC00461 |
| LINC00466 | LINC00486 | LINC00492 | LINC00511 | LINC00535 | LINC00536 |
| LINC00540 | LINC00582 | LINC00587 | LINC00595 | LINC00607 | LINC00623 |
| LINC00624 | LINC00639 | LINC00649 | LINC00683 | LINC00844 | LINC00861 |
| LINC00869 | LINC00871 | LINC00877 | LINC00880 | LINC00881 | LINC00882 |
| LINC00910 | LINC00922 | LINC00924 | LINC00927 | LINC00937 | LINC00941 |
| LINC00970 | LINC01006 | LINC01016 | LINC01019 | LINC01036 | LINC01065 |
| LINC01088 | LINC01090 | LINC01114 | LINC01117 | LINC01122 | LINC01135 |
| LINC01150 | LINC01170 | LINC01179 | LINC01189 | LINC01192 | LINC01197 |
| LINC01204 | LINC01205 | LINC01208 | LINC01221 | LINC01229 | LINC01252 |
| LINC01257 | LINC01278 | LINC01301 | LINC01307 | LINC01312 | LINC01320 |
| LINC01322 | LINC01331 | LINC01335 | LINC01346 | LINC01359 | LINC01392 |
| LINC01393 | LINC01399 | LINC01410 | LINC01412 | LINC01414 | LINC01424 |
| LINC01429 | LINC01436 | LINC01440 | LINC01476 | LINC01484 | LINC01500 |
| LINC01511 | LINC01517 | LINC01524 | LINC01533 | LINC01538 | LINC01550 |
| LINC01567 | LINC01572 | LINC01578 | LINC01594 | LINC01595 | LINC01605 |
| LINC01608 | LINC01625 | LINC01641 | LINC01673 | LINC01682 | LINC01694 |
| LINC01700 | LINC01719 | LINC01756 | LINC01775 | LINC01801 | LINC01837 |
| LINC01841 | LINC01844 | LINC01847 | LINC01861 | LINC01885 | LINC01893 |
| LINC01924 | LINC01928 | LINC01937 | LINC01944 | LINC01951 | LINC01954 |
| LINC01956 | LINC01978 | LINC01979 | LINC01989 | LINC01992 | LINC01994 |
| LINC02002 | LINC02028 | LINC02046 | LINC02097 | LINC02098 | LINC02112 |
| LINC02127 | LINC02133 | LINC02165 | LINC02203 | LINC02206 | LINC02208 |
| LINC02210- | LINC02215 | LINC02245 | LINC02250 | LINC02256 | LINC02284 |
| CRHR1 | | | | | |
| LINC02296 | LINC02299 | LINC02301 | LINC02306 | LINC02315 | LINC02326 |
| LINC02327 | LINC02334 | LINC02337 | LINC02340 | LINC02341 | LINC02342 |
| LINC02354 | LINC02355 | LINC02389 | LINC02422 | LINC02428 | LINC02447 |
| LINC02453 | LINC02469 | LINC02476 | LINC02485 | LINC02487 | LINC02511 |
| LINC02532 | LINC02539 | LINC02542 | LINC02549 | LINC02585 | LINC02606 |
| LINC02612 | LINC02615 | LINC02660 | LINC02710 | LINC02733 | LINC02757 |
| LINC02774 | LINC02780 | LINC02847 | LINC02853 | LINC02861 | LINC02865 |
| LINC02882 | LINC02884 | LINC02885 | LINGO1 | LINGO1-AS1 | LINGO2 |
| LIPC | LIPE-AS1 | LIPK | LIX1L-AS1 | LLPH | LMBR1 |
| LMCD1 | LMCD1-AS1 | LMF1 | LMNA | LMNTD2 | LMNTD2-AS1 |
| LMTK2 | LNCOC1 | LNCOG | LNX1 | LNX1-AS1 | LONP1 |
| LOXL1 | LPCAT3 | LPIN1 | LPIN2 | LPL | LPXN |
| LRAT | LRBA | LRCH1 | LRCH4 | LRGUK | LRIG2-DT |
| LRMDA | LRP1 | LRP2 | LRP4 | LRP8 | LRPPRC |
| LRRC15 | LRRC27 | LRRC37A17P | LRRC37A2 | LRRC37A4P | LRRC3B |
| LRRC45 | LRRC49 | LRRC4B | LRRC4C | LRRC56 | LRRC6 |
| LRRC63 | LRRC66 | LRRC73 | LRRC74A | LRRC74B | LRRC8C |
| LRRC9 | LRRFIP1 | LRRIQ4 | LRRN2 | LRRN4 | LRRTM2 |
| LRTM1 | LSAMP | LSM4 | LSMEM2 | LSP1 | LTF |
| LUC7L | LYNX1 | LYNX1-SLURP2 | LYRM4 | LYRM4-AS1 | LYSMD2 |
| LYST | LZTS3 | M6PR | MACF1 | MACO1 | MACROD1 |
| MAD1L1 | MADD | MAEA | MAFG | MAFTRR | MAGED1 |
| MAGI2 | MAGI3 | MAJIN | MAL2 | MAML3 | MAN1A2 |
| MAN1C1 | MAP1A | MAP2K1 | MAP2K2 | MAP2K5 | MAP2K7 |
| MAP3K11 | MAP3K13 | MAP3K14 | MAP3K19 | MAP3K2 | MAP3K20 |
| MAP3K4 | MAP3K7CL | MAP4K1 | MAP4K3 | MAP4K3-DT | MAP4K4 |
| MAP7 | MAPK14 | MAPK4 | MAPK8IP3 | MAPKAP1 | MAPKAPK5 |
| MAPRE2 | MAPT | MARCHF2 | MARCHF3 | MARK1 | MAST2 |
| MAST3 | MAST4 | MAT1A | MATK | MATN2 | MATN3 |
| MB21D2 | MBD3 | MBD5 | MBTPS1 | MCF2L | MCM10 |
| MCM8 | MCM8-AS1 | MCMDC2 | MCOLN1 | MCTP1 | MCTP2 |
| MCU | MDGA2 | MECOM | MED1 | MED13L | MED17 |
| MEF2B | MEG3 | MEGF11 | MEI4 | MEIKIN | MEIS2 |
| MELK | MEMO1 | MEP1AP4 | MERTK | METAP1D | METTL1 |
| METTL15 | METTL16 | METTL24 | METTL27 | METTL4 | METTL8 |
| MFAP1 | MFAP5 | MFNG | MFSD11 | MFSD12 | MFSD14C |
| MFSD4B | MFSD6 | MGAM | MGAT4A | MGAT5 | MGAT5B |
| MGMT | MGRN1 | MICAL1 | MICAL3 | MICALL2 | MICU1 |
| MICU2 | MIDN | MINDY1 | MINDY3 | MIP | MIPOL1 |
| MIR100HG | MIR1244-1 | MIR181A2HG | MIR325HG | MIR3659HG | MIR3681HG |
| MIR4307HG | MIR4422HG | MIR449C | MIR646HG | MIR6857 | MKNK2 |
| MKRN2OS | MLC1 | MLH1 | MLLT10 | MLXIPL | MLYCD |
| MMAB | MMD2 | MMEL1-AS1 | MMP19 | MNAT1 | MNT |
| MOB3A | MOK | MORN5 | MOV10 | MOV10L1 | MPHOSPH10 |
| MPHOSPH6P1 | MPHOSPH9 | MPP5 | MPPE1 | MPPED1 | MPV17L |
| MPZL3 | MRGPRF | MRM1 | MROH7 | MROH7-TTC4 | MRPL19 |
| MRPL33 | MRPL40 | MRPL45 | MRPL48 | MRPS22 | MRPS23 |
| MRPS25 | MRPS36 | MRPS6 | MRPS9-AS1 | MRRF | MRTFA |
| MS4A3 | MSANTD1 | MSANTD3 | MSANTD3- | MSH2 | MSH3 |
| | | | TMEFF1 | | |
| MSI2 | MSLN | MSR1 | MSRA | MSTRG.1003 | MSTRG.1007 |
| MSTRG.1033 | MSTRG.1035 | MSTRG.1036 | MSTRG.1048 | MSTRG.1049 | MSTRG.1062 |
| MSTRG.1066 | MSTRG.1111 | MSTRG.1113 | MSTRG.1121 | MSTRG.1132 | MSTRG.1142 |
| MSTRG.1174 | MSTRG.1248 | MSTRG.1280 | MSTRG.1333 | MSTRG.1337 | MSTRG.1351 |
| MSTRG.1392 | MSTRG.1402 | MSTRG.1441 | MSTRG.1469 | MSTRG.1487 | MSTRG.1496 |
| MSTRG.1519 | MSTRG.1536 | MSTRG.1537 | MSTRG.1539 | MSTRG.1562 | MSTRG.1632 |
| MSTRG.1633 | MSTRG.1634 | MSTRG.1635 | MSTRG.173 | MSTRG.1752 | MSTRG.1921 |
| MSTRG.1942 | MSTRG.1947 | MSTRG.198 | MSTRG.2014 | MSTRG.2046 | MSTRG.2047 |
| MSTRG.2059 | MSTRG.2104 | MSTRG.2106 | MSTRG.2107 | MSTRG.2109 | MSTRG.2119 |
| MSTRG.2122 | MSTRG.2140 | MSTRG.2148 | MSTRG.215 | MSTRG.2168 | MSTRG.2216 |
| MSTRG.2257 | MSTRG.2307 | MSTRG.2311 | MSTRG.2333 | MSTRG.2343 | MSTRG.2360 |
| MSTRG.2363 | MSTRG.237 | MSTRG.2378 | MSTRG.2397 | MSTRG.2417 | MSTRG.2444 |
| MSTRG.2476 | MSTRG.2527 | MSTRG.2559 | MSTRG.2573 | MSTRG.2585 | MSTRG.259 |
| MSTRG.2605 | MSTRG.2613 | MSTRG.2624 | MSTRG.2650 | MSTRG.2656 | MSTRG.2678 |
| MSTRG.2686 | MSTRG.2718 | MSTRG.2727 | MSTRG.2737 | MSTRG.2743 | MSTRG.2754 |
| MSTRG.2760 | MSTRG.2802 | MSTRG.2823 | MSTRG.2830 | MSTRG.2872 | MSTRG.2891 |
| MSTRG.2971 | MSTRG.2974 | MSTRG.2986 | MSTRG.3034 | MSTRG.3104 | MSTRG.3118 |
| MSTRG.3185 | MSTRG.3207 | MSTRG.3219 | MSTRG.3237 | MSTRG.3240 | MSTRG.3245 |
| MSTRG.327 | MSTRG.3285 | MSTRG.3311 | MSTRG.3345 | MSTRG.3396 | MSTRG.3423 |
| MSTRG.3440 | MSTRG.3455 | MSTRG.3476 | MSTRG.3481 | MSTRG.3501 | MSTRG.3534 |
| MSTRG.3536 | MSTRG.3602 | MSTRG.3603 | MSTRG.3618 | MSTRG.3634 | MSTRG.3642 |
| MSTRG.3658 | MSTRG.3685 | MSTRG.3707 | MSTRG.3733 | MSTRG.3736 | MSTRG.3809 |
| MSTRG.3836 | MSTRG.3855 | MSTRG.3861 | MSTRG.3867 | MSTRG.3874 | MSTRG.3884 |
| MSTRG.3909 | MSTRG.3922 | MSTRG.3938 | MSTRG.397 | MSTRG.3970 | MSTRG.3996 |
| MSTRG.4040 | MSTRG.4106 | MSTRG.4142 | MSTRG.4156 | MSTRG.4174 | MSTRG.4176 |
| MSTRG.4178 | MSTRG.4183 | MSTRG.4188 | MSTRG.4189 | MSTRG.4190 | MSTRG.4191 |
| MSTRG.42 | MSTRG.4201 | MSTRG.4205 | MSTRG.4218 | MSTRG.4219 | MSTRG.4227 |
| MSTRG.4233 | MSTRG.4273 | MSTRG.4349 | MSTRG.4417 | MSTRG.4463 | MSTRG.4499 |
| MSTRG.458 | MSTRG.4610 | MSTRG.4624 | MSTRG.4632 | MSTRG.4689 | MSTRG.4747 |
| MSTRG.4761 | MSTRG.482 | MSTRG.4826 | MSTRG.4851 | MSTRG.4856 | MSTRG.4861 |
| MSTRG.4870 | MSTRG.4874 | MSTRG.4880 | MSTRG.4953 | MSTRG.4990 | MSTRG.500 |
| MSTRG.5008 | MSTRG.5031 | MSTRG.5092 | MSTRG.5123 | MSTRG.5128 | MSTRG.5130 |
| MSTRG.5137 | MSTRG.5138 | MSTRG.5147 | MSTRG.5154 | MSTRG.518 | MSTRG.5209 |
| MSTRG.53 | MSTRG.5326 | MSTRG.5339 | MSTRG.5350 | MSTRG.5358 | MSTRG.5368 |
| MSTRG.5375 | MSTRG.5410 | MSTRG.5441 | MSTRG.5573 | MSTRG.5594 | MSTRG.5686 |
| MSTRG.5694 | MSTRG.5707 | MSTRG.5862 | MSTRG.589 | MSTRG.599 | MSTRG.603 |
| MSTRG.620 | MSTRG.649 | MSTRG.654 | MSTRG.667 | MSTRG.710 | MSTRG.734 |
| MSTRG.797 | MSTRG.998 | MTA3 | MTAP | MTARC2 | MTBP |
| MTCH2 | MTCO1P28 | MTCO3P13 | MTDH | MTFR1 | MTFR2P2 |
| MTHFD1 | MTHFD1L | MTHFD2 | MTMR1 | MTMR12 | MTMR14 |
| MTND1P22 | MTND2P13 | MTREX | MTRF1 | MTURN | MTUS1 |
| MTUS2 | MUC17 | MUC3A | MUC5AC | MUC5B | MUC6 |
| MUC7 | MVB12A | MYBPHL | MYDGF | MYH10 | MYH14 |
| MYH16 | MYLK | MYLK2 | MYO10 | MYO15A | MYO16 |
| MYO1B | MYO1D | MYOIF | MYO3A | MYO3B | MYO5A |
| MYO5B | MYO7A | MYO7B | MYO9A | MYOF | MYOM1 |
| MYOM2 | MYOM3 | MYOSLID | MYPN | MYRF | MYSM1 |
| N4BP2 | NAA25 | NAALADL2 | NAALADL2-AS3 | NADSYN1 | NAIP |
| NAIPP1 | NALCN | NALCN-AS1 | NAP1L4 | NARS2 | NASP |
| NAT2 | NAV2 | NBEA | NBN | NBPF1 | NBPF10 |
| NBPF15 | NBPF20 | NBPF4 | NCALD | NCAN | NCAPH |
| NCF1 | NCF1B | NCF1C | NCKAP1L | NCKIPSD | NCMAP |
| NCOA1 | NCOA6 | NCOR1 | NCR3LG1 | NDE1 | NDEL1 |
| NDFIP1 | NDRG3 | NDUFA10 | NDUFA13 | NDUFA4L2 | NDUFA6-DT |
| NDUFA9 | NDUFB3 | NDUFC2- | NDUFS2 | NEB | NEBL |
| | | KCTD14 | | | |
| NECTIN1 | NECTIN2 | NECTIN3 | NEDD9 | NEIL2 | NEK11 |
| NEK4 | NEK6 | NEK8 | NELFA | NEMP2 | NEUROG3 |
| NF1P2 | NFAM1 | NFASC | NFAT5 | NFATC1 | NFATC2IP |
| NFIA | NFIX | NFU1 | NFX1 | NGEF | NGFR |
| NGLY1 | NHS | NHSL1 | NHSL2 | NIBAN1 | NIM1K |
| NINJ2 | NINJ2-AS1 | NINL | NIPAL1 | NIPAL2 | NIPBL |
| NIPSNAP2 | NISCH | NKAIN1 | NKAIN2 | NKAIN3 | NKD1 |
| NLGN1 | NLK | NLRC4 | NLRP1 | NLRP2 | NLRP6 |
| NLRX1 | NME3 | NME7 | NMNAT2 | NMT2 | NOL10 |
| NOL4L | NOMO1 | NOP14-AS1 | NOP2 | NOS1 | NOS2P1 |
| NOS3 | NOSIP | NOTCH4 | NOX5 | NOXO1 | NPAS1 |
| NPAS2 | NPC1L1 | NPEPPS | NPHP1 | NPHS1 | NPIPA1 |
| NPIPA8 | NPIPB8 | NPLOC4 | NPM1 | NPRL3 | NPSR1 |
| NPSR1-AS1 | NQO2 | NR1D2 | NR1H2 | NR2F1-AS1 | NR3C2 |
| NR4A1 | NR5A1 | NR6A1 | NRAP | NRDC | NRG1 |
| NRG2 | NRP2 | NRXN1 | NRXN2 | NRXN3 | NSF |
| NSG2 | NSL1 | NSUN4 | NSUN5 | NSUN6 | NSUN7 |
| NTN4 | NTRK1 | NTRK2 | NTRK3 | NUDC | NUDT5 |
| NUFIP1 | NUMBL | NUP107 | NUP133 | NUP210 | NUP85 |
| NUP98 | NUTM2B-AS1 | NWD1 | NXF2 | NXN | OAZ1 |
| OBI1-AS1 | OBSCN | OCLN | OCLNP1 | ODF2L | OFCC1 |
| OGG1 | OIP5-AS1 | OIT3 | OLA1 | OPA1 | OPA1-AS1 |
| OPA3 | OPALIN | OPRM1 | OPTN | OR10AH1P | OR10K1 |
| OR1G1 | OR1N2 | OR2B6 | OR2J3 | OR2T2 | OR4D1 |
| OR4M2 | OR52E5 | OR7A10 | OR7A8P | OR7D2 | OR7E161P |
| OR9H1P | OR9S24P | ORAI1 | ORC3 | OSBP2 | OSBPL10 |
| OSBPL10-AS1 | OSBPL1A | OSBPL8 | OSBPL9 | OSMR-AS1 | OTOGL |
| OVAAL | OVCH1 | OVCH1-AS1 | OVOL2 | P2RX4 | P2RX5 |
| P2RX5- | P4HA3 | P4HTM | PA2G4 | PAAF1 | PACS1 |
| TAX1BP3 | | | | | |
| PACSIN2 | PADI1 | PAFAH1B1 | PAGR1 | PAK4 | PALM2AKAP2 |
| PAN3 | PAPOLG | PAPPA | PAPPA2 | PAQR5 | PARD3 |
| PARD3B | PARGP1 | PARL | PARN | PARP15 | PARP16 |
| PARP4P1 | PARP4P2 | PARP6 | PARPBP | PARVA | PASD1 |
| PASK | PATE4 | PATJ | PAWR | PAX2 | PAX5 |
| PAXIP1 | PBRM1 | PBX3 | PC | PCAT1 | PCAT14 |
| PCAT4 | PCBP1-AS1 | PCBP3 | PCCA | PCDH11X | PCDH15 |
| PCDH8 | PCDHA1 | PCDHA10 | PCDHA11 | PCDHA12 | PCDHA13 |
| PCDHA2 | PCDHA3 | PCDHA4 | PCDHA5 | PCDHA6 | PCDHA7 |
| PCDHA8 | PCDHA9 | PCDHAC1 | PCDHAC2 | PCDHGA1 | PCDHGA2 |
| PCDHGA3 | PCDHGA4 | PCDHGA5 | PCDHGA6 | PCDHGA7 | PCDHGA8 |
| PCDHGA9 | PCDHGB1 | PCDHGB2 | PCDHGB3 | PCDHGB4 | PCDHGB5 |
| PCGF3 | PCID2 | PCNT | PCNX3 | PCP2 | PCSK2 |
| PCSK5 | PCYT1B | PDCD1LG2 | PDCD6 | PDCD6-AHRR | PDE10A |
| PDE11A | PDE1A | PDE4A | PDE4D | PDE4DIP | PDE6B |
| PDE6B-AS1 | PDE7A | PDE8B | PDGFA | PDHX | PDIA2 |
| PDIA4 | PDP1 | PDPK1 | PDXDC1 | PDXK | PDXP |
| PDZD2 | PDZD9 | PDZK1 | PEAK1 | PELI1 | PELI2 |
| PELP1 | PEPD | PES1 | PEX13 | PEX14 | PEX5L |
| PFKFB3 | PFKP | PGAP6 | PGM1 | PGM2 | PHACTR1 |
| PHACTR2 | PHACTR4 | PHC2 | PHF12 | PHF19 | PHF2 |
| PHF21A | PHIP | PHKB | PHLPP1 | PHOSPHO1 | PHTF1 |
| PHYH | PI4KAP2 | PIAS1 | PIAS4 | PICALM | PID1 |
| PIDD1 | PIEZO2 | PIGG | PIGL | PIGN | PIGQ |
| PIK3AP1 | PIK3C2B | PIK3C2G | PIK3CB | PIK3IP1-DT | PIK3R6 |
| PIP4K2A | PIPOX | PITPNA | PITPNC1 | PITPNM2 | PITPNM3 |
| PITRM1 | PITRM1-AS1 | PIWIL2 | PKD1 | PKD1P1 | PKD2L2 |
| PKNOX1 | PKP1 | PKP2 | PLA2G12B | PLA2G4A | PLA2G4D |
| PLA2R1 | PLAA | PLAAT1 | PLB1 | PLBD1 | PLCE1 |
| PLCG2 | PLCH1 | PLCH2 | PLCL2 | PLCXD1 | PLCXD3 |
| PLD1 | PLEKHA7 | PLEKHA8 | PLEKHA8P1 | PLEKHB2 | PLEKHD1 |
| PLEKHG1 | PLEKHG2 | PLEKHG5 | PLEKHH1 | PLEKHJ1 | PLEKHM1 |
| PLEKHM3 | PLEKHO2 | PLGRKT | PLIN3 | PLIN4 | PLK5 |
| PLP1 | PLUT | PLVAP | PLXDC1 | PLXNA4 | PM20D2 |
| PMM2 | PMS2P10 | PMS2P7 | PNKD | PNPLA6 | PNPT1 |
| POC5 | PODXL | POGZ | POLA2 | POLE | POLR1C |
| POLR2A | POLR2J4 | POLR3B | POLRMT | POMZP3 | POR |
| POTEF | POTEJ | POU2F2 | POU5F1B | POU6F1 | PP7080 |
| PPARA | PPARGC1B | PPFIA1 | PPFIA2 | PPFIA3 | PPFIBP1 |
| PPHLN1 | PPIAP77 | PPIG | PPIP5K1 | PPM1A | PPM1B |
| PPM1E | PPM1H | PPME1 | PPP1CA | PPP1CB | PPP1R11 |
| PPP1R12A | PPP1R12B | PPP1R12C | PPP1R14C | PPP1R2 | PPP1R7 |
| PPP1R9A | PPP2R1A | PPP2R2A | PPP2R2D | PPP2R5D | PPP2R5E |
| PPP3R1 | PPP4C | PPP4R3B | PPP4R4 | PPP5D1 | PPP6C |
| PPTC7 | PRAMEF6 | PRANCR | PRCC | PRCP | PRDM8 |
| PRELID2 | PRELP | PREP | PRH1 | PRICKLE1 | PRIM2 |
| PRIMA1 | PRKAG2 | PRKAR1B | PRKAR2A | PRKCA | PRKCE |
| PRKCH | PRKCZ | PRKD1 | PRKDC | PRKN | PRLHR |
| PRMT1 | PRMT8 | PRMT9 | PRNT | PROX1-AS1 | PRPF18 |
| PRPF39 | PRPF40B | PRR11 | PRR13 | PRR14L | PRR33 |
| PRR5 | PRR5-ARHGAP8 | PRR5L | PRRG2 | PRSS23 | PRSS57 |
| PRTN3 | PSCA | PSD3 | PSEN1 | PSG11 | PSG2 |
| PSG8 | PSMA1 | PSMA8 | PSMB2 | PSMB7 | PSMD8 |
| PSME4 | PSMF1 | PSMG2 | PSMG4 | PSTPIP1 | PTAFR |
| PTBP3 | PTCD1 | PTDSS2 | PTGR2 | PTK2 | PTMA |
| PTN | PTP4A3 | PTPN2 | PTPRA | PTPRC | PTPRF |
| PTPRH | PTPRM | PTPRN2 | PTPRS | PTPRT | PTRH2 |
| PUDPP2 | PUM2 | PUM3 | PUS1 | PVALEF | PVR |
| PWRN1 | PXDN | PXDNL | PXMP2 | PXT1 | PYCR3 |
| PYGO1 | PYY | QSER1 | R3HCC1 | R3HDM2 | RAB10 |
| RAB11A | RAB11FIP3 | RAB11FIP4 | RAB17 | RAB18 | RAB20 |
| RAB23 | RAB26 | RAB28 | RAB2A | RAB31 | RAB37 |
| RAB39B | RAB3B | RAB3C | RAB3D | RAB3GAP1 | RAB3IL1 |
| RAB3IP | RAB40C | RAB44 | RAB6A | RAB6B | RABEP1 |
| RABEP2 | RABGAP1L | RABGAP1L-AS1 | RAC2 | RAD17 | RAD18 |
| RAD50 | RAD51B | RAD52 | RAD54L2 | RADIL | RAF1 |
| RALA | RALGAPA2 | RALY | RALYL | RAMP1 | RANBP17 |
| RANBP9 | RAP1A | RAP1GAP2 | RAP1GDS1 | RASD1 | RASEF |
| RASGRF1 | RASSF2 | RASSF4 | RASSF6 | RASSF8 | RAVER2 |
| RBFOX3 | RBL1 | RBM14-RBM4 | RBM18 | RBM19 | RBM33 |
| RBM47 | RBM5 | RBM6 | RBMS1 | RBMY1A1 | RBMY1B |
| RBP7 | RBPJ | RBX1 | RCHY1 | RCOR2 | RDH13 |
| RDH8 | RDM1P5 | RECQL | REEP6 | RELN | REPS1 |
| RERE | REREP1Y | REREP2Y | REXO1 | REXO1L10P | RFLNA |
| RFPL1S | RFX1 | RFX2 | RFX3 | RFX3-AS1 | RGPD8 |
| RGS14 | RGS22 | RGS3 | RGS5 | RGS6 | RHBDD1 |
| RHBDD2 | RHBDF1 | RHCE | RHOQ | RHOQ-AS1 | RHPN1 |
| RIC8B | RIDA | RIMBP2 | RIMKLA | RIMS1 | RIMS4 |
| RINT1 | RIOK1 | RIPOR2 | RIT1 | RMDN2 | RMDN2-AS1 |
| RMI2 | RMND5A | RN7SKP58 | RN7SL442P | RN7SL498P | RN7SL678P |
| RNA18S4 | RNA28S4 | RNA45S4 | RNASEH1 | RNASEH2B-AS1 | RNASET2 |
| RNF103-CHMP3 | RNF111 | RNF115 | RNF126 | RNF130 | RNF144A |
| RNF165 | RNF182 | RNF19B | RNF213 | RNF213-AS1 | RNF214 |
| RNF216 | RNF217-AS1 | RNF24 | RNF38 | RNF4 | RNF43 |
| RNFT1 | RNFT2 | RNGTT | RNPEPL1 | RNU6-1206P | ROBO2 |
| ROCK1 | ROCK1P1 | ROCK2 | RORA | RORA-AS2 | RP1L1 |
| RP2 | RPA1 | RPA3 | RPARP-AS1 | RPH3AL | RPL12P13 |
| RPL17-C18orf32 | RPL36AP39 | RPL5 | RPN2 | RPS10-NUDT3 | RPS12P3 |
| RPS16 | RPS4XP2 | RPS6KA2 | RPS6KB1 | RPS6KC1 | RPTN |
| RPUSD1 | RRAS2 | RRN3P2 | RRP12 | RRP15 | RRP7BP |
| RSF1 | RSL1D1 | RSPH14 | RSPH6A | RSPH9 | RSRC1 |
| RSRC2 | RSU1 | RSU1P2 | RTL1 | RTTN | RUFY4 |
| RUNX3 | RUVBL1 | RYBP | RYK | RYR2 | RYR3 |
| SACM1L | SAMD11 | SAMD12 | SAMD12-AS1 | SAMD5 | SAP130 |
| SAP30L-AS1 | SARAF | SARNP | SATB1 | SATL1 | SAXO1 |
| SAXO2 | SBF2 | SBF2-AS1 | SBK1 | SBNO2 | SBSN |
| SBSPON | SCAF11 | SCAI | SCAPER | SCARA3 | SCARA5 |
| SCARB1 | SCARF1 | SCFD1 | SCFD2 | SCGB2B2 | SCMH1 |
| SCML4 | SCN11A | SCN3A | SCN8A | SCNN1A | SCNN1B |
| SCP2 | SCRN1 | SCTR | SCYL1 | SCYL2 | SDC2 |
| SDHAF2 | SDHD | SDK1 | SDR42E1 | SDS | SEC14L1 |
| SEC14L3 | SEC22B4P | SEC22C | SEC24B-AS1 | SEH1L | SELENOI |
| SELENOP | SELENOT | SEM1 | SEMA3G | SEMA4B | SEMA4F |
| SEMA5A | SEMA5B | SEMA6D | SEPHS1 | SEPTIN1 | SEPTIN10 |
| SEPTIN12 | SEPTIN14 | SEPTIN14P1 | SERF1A | SERGEF | SERHL |
| SERHL2 | SERINC2 | SERINC5 | SERP1 | SERPINA6 | SERPINB1 |
| SERPINB8 | SERTAD2 | SETBP1 | SETD1B | SETD4 | SETD5 |
| SEZ6L | SEZ6L2 | SEZ6L-AS1 | SFMBT1 | SFMBT2 | SFPQP1 |
| SFRP1 | SFSWAP | SFTPA1 | SFTPA2 | SFXN1 | SFXN2 |
| SFXN5 | SGCA | SGF29 | SGK1 | SGK3 | SGMS1 |
| SGO1 | SGO2 | SGSM1 | SGSM2 | SGTA | SH2D3A |
| SH3BP2 | SH3D19 | SH3GL3 | SH3KBP1 | SH3PXD2B | SH3RF1 |
| SH3RF3 | SH3TC2 | SH3YL1 | SHANK2 | SHC2 | SHF |
| SHISAL1 | SHISAL2B | SHLD1 | SHMT1 | SHOC1 | SHOX2 |
| SHQ1 | SHROOM3 | SHROOM3-AS1 | SHROOM4 | SHTN1 | SI |
| SIAH1 | SIGLEC1 | SIK3 | SIL1 | SIM2 | SIMC1 |
| SIN3B | SIPA1 | SIPA1L3 | SIRT1 | SIRT5 | SIRT7 |
| SKAP1 | SKAP1-AS1 | SKAP2 | SKI | SKP1 | SLAIN2 |
| SLBP | SLC10A1 | SLC11A2 | SLC12A9 | SLC14A1 | SLC14A2 |
| SLC16A4 | SLC16A7 | SLC16A8 | SLC19A1 | SLC19A2 | SLC1A3 |
| SLC1A5 | SLC1A6 | SLC1A7 | SLC22A23 | SLC23A2 | SLC24A3 |
| SLC25A10 | SLC25A19 | SLC25A21 | SLC25A32 | SLC25A41 | SLC25A6 |
| SLC26A11 | SLC26A8 | SLC29A2 | SLC2A12 | SLC2A13 | SLC2A9 |
| SLC30A3 | SLC30A7 | SLC30A9 | SLC35E2B | SLC35E3 | SLC35E4 |
| SLC35F1 | SLC35F2 | SLC37A3 | SLC38A10 | SLC38A9 | SLC39A10 |
| SLC39A8 | SLC3A1 | SLC41A3 | SLC44A2 | SLC45A4 | SLC46A2 |
| SLC47A2 | SLC4A10 | SLC4A11 | SLC4A5 | SLC5A1 | SLC5A11 |
| SLC66A1L | SLC66A3 | SLC6A16 | SLC6A19 | SLC7A10 | SLC7A9 |
| SLC8A3 | SLC8B1 | SLC9A3 | SLC9A9 | SLC9B1 | SLC9B1P4 |
| SLCO1B3 | SLCO2A1 | SLCO2B1 | SLCO3A1 | SLCO5A1 | SLFN12L |
| SLIT3 | SLITRK2 | SLMAP | SLURP2 | SLX4IP | SMAD3 |
| SMAP2 | SMARCA2 | SMARCA4 | SMARCC1 | SMARCD3 | SMC1A |
| SMC1B | SMG1 | SMG1P4 | SMG5 | SMG6 | SMIM11A |
| SMIM14 | SMIM22 | SMIM24 | SMIM4 | SMN1 | SMOC1 |
| SMOC2 | SMOX | SMPD2 | SMPD3 | SMPD4P1 | SMTN |
| SMYD3 | SMYD4 | SMYD5 | SNAPC3 | SNCAIP | SND1 |
| SNED1 | SNHG14 | SNHG31 | SNORC | SNRK | SNRNP200 |
| SNRNP35 | SNRNP40 | SNRNP70 | SNRPF | SNRPN | SNTA1 |
| SNTG1 | SNU13 | SNUPN | SNX14 | SNX27 | SNX29 |
| SNX29P2 | SNX30 | SNX31 | SNX32 | SNX5 | SNX7 |
| SNX8 | SNX9 | SOCAR | SOD2 | SOD2-OT1 | SORBS1 |
| SORBS2 | SORD | SORL1 | SOX1-OT | SOX2-OT | SOX5 |
| SOX6 | SOX9-AS1 | SP1 | SP140 | SPACA7 | SPAG16 |
| SPAG5 | SPAG6 | SPAST | SPATA13 | SPATA22 | SPATA31C1 |
| SPATA31E2P | SPATS2 | SPATS2L | SPC25 | SPDYA | SPDYE3 |
| SPECC1 | SPECC1L- | SPECC1P1 | SPEN | SPESP1 | SPI1 |
| | ADORA2A | | | | |
| SPIDR | SPINDOC | SPIRE1 | SPO11 | SPOCK2 | SPON1 |
| SPON2 | SPPL3 | SPRED2 | SPRY3 | SPRY4-AS1 | SPSB1 |
| SPSB3 | SPTBN1 | SPTLC2 | SQOR | SRBD1 | SRCAP |
| SRCIN1 | SREBF2 | SREK1 | SREK1IP1 | SRF | SRGAP1 |
| SRGAP2B | SRP68 | SRPK1 | SRR | SRRM2-AS1 | SRRM3 |
| SRRT | SRSF2 | SRSF3 | SRSF4 | SS18L1 | SSBP2 |
| SSBP3 | SSBP4 | SSH3 | SSR1 | SSU72 | ST14 |
| ST18 | ST3GAL1 | ST3GAL3 | ST3GAL6-AS1 | ST6GALNAC3 | ST7L |
| ST8SIA4 | ST8SIA6 | STAG3L2 | STAG3L3 | STAM | STARD10 |
| STARD13 | STAT1 | STAT3 | STAT6 | STAU1 | STEAP1B |
| STIM1 | STIM2 | STIMATE | STIMATE- | STK10 | STK11 |
| | | | MUSTN1 | | |
| STK24 | STK3 | STK32B | STK32C | STK33 | STK39 |
| STK40 | STON1 | STON1- | STON2 | STPG2 | STPG4 |
| | | GTF2A1L | | | |
| STRA6LP | STRADB | STRC | STRIP1 | STRN | STRN4 |
| STS | STUM | STX12 | STX17-AS1 | STX6 | STX7 |
| STX8 | STXBP5 | STXBP5-AS1 | SUCLG2 | SUGT1 | SULT1A1 |
| SULT1B1 | SULT1C2P1 | SULT1C3 | SULT4A1 | SULT6B1 | SUMF1 |
| SUN1 | SUPT3H | SUPT5H | SUSD1 | SUSD4 | SUZ12 |
| SV2B | SV2C | SVEP1 | SVIL-AS1 | SYCP2L | SYK |
| SYNDIG1 | SYNE2 | SYNGAP1 | SYNPO2 | SYT1 | SYT14 |
| SYT17 | TACC3 | TAF1B | TAF3 | TAF4 | TAF6 |
| TAF6L | TALDO1 | TANGO2 | TARID | TAS2R14 | TASP1 |
| TAX1BP1 | TBC1D1 | TBC1D10C | TBC1D14 | TBC1D16 | TBC1D19 |
| TBC1D22B | TBC1D32 | TBC1D5 | TBC1D8 | TBCA | TBCE |
| TBCK | TBL3 | TBX1 | TBXA2R | TCAM1P | TCEA3 |
| TCEANC2 | TCERG1L | TCF20 | TCF21 | TCF3 | TCF7 |
| TCF7L2 | TCIRG1 | TCL1B | TCTE1 | TCTN1 | TCTN2 |
| TCTN3 | TDO2 | TDRD12 | TDRD5 | TEC | TECPR1 |
| TECRL | TEF | TELO2 | TEMN3-AS1 | TENM2 | TENM3 |
| TENM3-AS1 | TENM4 | TENT2 | TENT4A | TENT4B | TEPSIN |
| TERB1 | TERF2 | TERF2IP | TESC | TEX10 | TEX14 |
| TEX264 | TEX49 | TF | TFAP2C | TFAP4 | TFCP2 |
| TGFB1 | TGFBR2 | TGFBR3 | TGM4 | THADA | THAP2 |
| THAP6 | THBS3 | THEG | THOC3 | THOC7 | THOP1 |
| THRAP3 | THSD4 | THSD7B | TIA1 | TICAM1 | TIGD6 |
| TIMELESS | TIMM29 | TIMM44 | TIMP2 | TJP3 | TK1 |
| TKFC | TLCD4 | TLCD4-RWDD3 | TLE1 | TLE2 | TLE4 |
| TLK2 | TLL2 | TLN1 | TLR1 | TLR6 | TM2D1 |
| TM2D3 | TM4SF18 | TM4SF5 | TM9SF4 | TMC4 | TMCC1 |
| TMCO4 | TMED10 | TMED4 | TMEM105 | TMEM116 | TMEM120B |
| TMEM123 | TMEM131 | TMEM132B | TMEM135 | TMEM138 | TMEM143 |
| TMEM145 | TMEM14B | TMEM151B | TMEM163 | TMEM170B | TMEM184A |
| TMEM184B | TMEM184C | TMEM192 | TMEM211 | TMEM220 | TMEM220-AS1 |
| TMEM221 | TMEM223 | TMEM225B | TMEM231 | TMEM234 | TMEM241 |
| TMEM242 | TMEM245 | TMEM258 | TMEM259 | TMEM260 | TMEM268 |
| TMEM273 | TMEM39A | TMEM43 | TMEM44 | TMEM45B | TMEM59L |
| TMEM63C | TMEM72-AS1 | TMLHE | TMLHE-AS1 | TMPRSS12 | TMPRSS6 |
| TMPRSS9 | TMSB15B | TMSB15B-AS1 | TMTC1 | TNC | TNFAIP8 |
| TNFRSF10A | TNFRSF11A | TNFRSF11B | TNFSF11 | TNK1 | TNKS2-AS1 |
| TNNT1 | TNNT3 | TNPO3 | TNR | TNRC18 | TNRC6A |
| TNRC6B | TNRC6C | TNS1 | TNS3 | TOLLIP | TOP1MT |
| TOP2B | TOP3A | TOPBP1 | TOR1AIP2 | TOX2 | TP53 |
| TP53BP1 | TPCN1 | TPCN2 | TPD52 | TPH1 | TPM4 |
| TPMT | TPPP | TPRKB | TPTE2P2 | TPTE2P6 | TPTEP2 |
| TPTEP2- | TRAF3IP2-AS1 | TRAF7 | TRAM2-AS1 | TRANK1 | TRAP1 |
| CSNK1E | | | | | |
| TRAPPC3 | TRAPPC8 | TRAPPC9 | TRDN | TRDN-AS1 | TRERF1 |
| TRIB3 | TRIM16L | TRIM37 | TRIM5 | TRIM50 | TRIM58 |
| TRIM65 | TRIM66 | TRIM69 | TRIM71 | TRIP10 | TRIP12 |
| TRIR | TRMT11 | TRMT1L | TRMT44 | TRNT1 | TRPC2 |
| TRPC4 | TRPM1 | TRPM2 | TRPM3 | TRPM4 | TRPM7 |
| TRPS1 | TRPV1 | TRPV4 | TRRAP | TSC2 | TSEN15 |
| TSEN2 | TSG101 | TSGA10 | TSHZ1 | TSNARE1 | TSNAX |
| TSNAX-DISC1 | TSPAN13 | TSPAN15 | TSPAN16 | TSPAN32 | TSPAN4 |
| TSPAN8 | TSPEAR | TSPOAP1 | TSPOAP1-AS1 | TSPY3 | TTBK2 |
| TTC21A | TTC21B | TTC21B-AS1 | TTC25 | TTC26 | TTC27 |
| TTC28 | TTC3 | TTC33 | TTC34 | TTC39B | TTC7A |
| TTLL11 | TTLL11-IT1 | TTLL4 | TTLL6 | TTLL8 | TTLL9 |
| TTN | TTN-AS1 | TTTY4B | TTYH2 | TUBA3GP | TUBB2B |
| TUBB6 | TUBB8P5 | TUBGCP3 | TVP23C | TVP23C-CDRT4 | TXLNG |
| TXNDC11 | TXNRD1 | TXNRD2 | TXNRD3 | TYW1B | UBA3 |
| UBAC2 | UBAC2-AS1 | UBAP2 | UBE2A | UBE2D2 | UBE2D3 |
| UBE2F | UBE2F-SCLY | UBE2G1 | UBE2G2 | UBE2J1 | UBE2K |
| UBE2L3 | UBE2O | UBE2R2 | UBE2R2-AS1 | UBE2S | UBE3D |
| UBN2 | UBOX5-AS1 | UBQLN4 | UBR5 | UBTD1 | UBXN2A |
| UBXN6 | UCP3 | UEVLD | UGGT2 | UGP2 | UHRF1 |
| UHRF1BP1L | UHRF2 | UIMC1 | ULK2 | ULK4 | UMAD1 |
| UNC13A | UNC13B | UNC13C | UNC13D | UNC5B | UNC5C |
| UNC93B2 | UNKL | UPF1 | UPF2 | UPF3AP1 | UPK1A |
| UPK1A-AS1 | UPP2 | UQCC1 | UQCR11 | URGCP | URGCP-MRPS24 |
| URI1 | UROC1 | USE1 | USH2A | USP12 | USP15 |
| USP24 | USP2-AS1 | USP31 | USP33 | USP34 | USP36 |
| USP39 | USP4 | USP42 | USP44 | USP45 | USP48 |
| USP54 | USP6NL | UST | UTRN | VASN | VAT1L |
| VAV1 | VAV3 | VBP1 | VCL | VCPIP1 | VEPH1 |
| VEZT | VGLL4 | VIPR1 | VIPR1-AS1 | VIT | VMAC |
| VOPP1 | VPS13A | VPS13B | VPS13B-DT | VPS13C | VPS26A |
| VPS35L | VPS37B | VPS39 | VPS50 | VPS53 | VPS54 |
| VRK1 | VRK3 | VRTN | VSTM4 | VTA1 | VTCN1 |
| VTI1A | VWA3B | VWA7 | VWF | WAKMAR2 | WASF1 |
| WASF2 | WBP1LP5 | WDFY3 | WDFY4 | WDPCP | WDR12 |
| WDR31 | WDR41 | WDR49 | WDR59 | WDR6 | WDR62 |
| WDR7 | WDR7-OT1 | WDR86-AS1 | WDR88 | WDR90 | WDTC1 |
| WFDC10B | WFDC11 | WFDC3 | WFDC8 | WIPF1 | WIPI2 |
| WNK3 | WNT10A | WNT2B | WNT3 | WNT7B | WNT8B |
| WNT9A | WRAP53 | WRAP73 | WWOX | XAB2 | XBP1 |
| XG | XGY1 | XIRP2 | XK | XKR4 | XKR5 |
| XKR6 | XKR7 | XPNPEP1 | XPO1 | XPO5 | XPO7 |
| XPR1 | XRCC1 | XRRA1 | XXYLT1 | XYLB | XYLT1 |
| YAF2 | YARS1 | YBX2 | YEATS4 | YIPF2 | YIPF4 |
| YJEFN3 | YLPM1 | YPEL1 | YPEL2 | Y_RNA | YTHDF2 |
| YWHAE | Z82190.2 | Z83844.2 | Z84466.1 | Z84723.1 | Z94160.1 |
| Z94721.2 | Z96074.1 | Z97634.1 | Z98883.1 | ZAN | ZBBX |
| ZBTB16 | ZBTB44 | ZBTB7A | ZBTB7C | ZC3H10 | ZC3H13 |
| ZC3H14 | ZC3H3 | ZC3H4 | ZC3HAV1 | ZC3HAV1L | ZC3HC1 |
| ZCCHC17 | ZCCHC24 | ZCCHC7 | ZCCHC9 | ZCRB1 | ZDHHC11 |
| ZDHHC14 | ZDHHC15 | ZDHHC20 | ZDHHC24 | ZDHHC3 | ZEB1 |
| ZFAND3 | ZFC3H1 | ZFP41 | ZFPM2 | ZFPM2-AS1 | ZFR2 |
| ZFYVE28 | ZFYVE9 | ZKSCAN7 | ZKSCAN7-AS1 | ZMAT4 | ZMYM1 |
| ZMYND8 | ZNF100 | ZNF106 | ZNF124 | ZNF131 | ZNF136 |
| ZNF140 | ZNF141 | ZNF146 | ZNF195 | ZNF208 | ZNF224 |
| ZNF225 | ZNF226 | ZNF232 | ZNF235 | ZNF236 | ZNF248 |
| ZNF263 | ZNF266 | ZNF282 | ZNF284 | ZNF302 | ZNF316 |
| ZNF318 | ZNF337-AS1 | ZNF33A | ZNF33B | ZNF350-AS1 | ZNF362 |
| ZNF365 | ZNF385A | ZNF385B | ZNF394 | ZNF404 | ZNF407 |
| ZNF423 | ZNF438 | ZNF44 | ZNF461 | ZNF48 | ZNF483 |
| ZNF490 | ZNF496 | ZNF500 | ZNF516 | ZNF521 | ZNF528 |
| ZNF536 | ZNF540 | ZNF554 | ZNF556 | ZNF557 | ZNF559-ZNF177 |
| ZNF562 | ZNF564 | ZNF565 | ZNF566 | ZNF569 | ZNF573 |
| ZNF578 | ZNF585A | ZNF609 | ZNF615 | ZNF624 | ZNF638 |
| ZNF682 | ZNF701 | ZNF702P | ZNF704 | ZNF705CP | ZNF706 |
| ZNF709 | ZNF713 | ZNF718 | ZNF721 | ZNF724 | ZNF727 |
| ZNF728 | ZNF766 | ZNF775 | ZNF782 | ZNF785 | ZNF789 |
| ZNF790-AS1 | ZNF804B | ZNF808 | ZNF816- | ZNF826P | ZNF83 |
| | | | ZNF321P | | |
| ZNF836 | ZNF862 | ZNF880 | ZNF883 | ZNF92 | ZNF962P |
| ZNF99 | ZNRF2P2 | ZNRF3 | ZRANB1 | ZRANB3 | ZSCAN10 |
| ZSWIM4 | ZSWIM5 | ZYG11A | | | |

The probes may be nucleic acid molecules (e.g., RNA or DNA) having sequence complementarity with nucleic acid sequences (e.g., RNA or DNA) of the one or more genomic loci (e.g., liver disease-associated genomic loci). These nucleic acid molecules may be primers or enrichment sequences. The assaying of the cell-free biological sample using probes that are selective for the one or more genomic loci (e.g., liver disease-associated genomic loci) may comprise use of array hybridization (e.g., microarray-based), PCR, or nucleic acid sequencing (e.g., RNA sequencing or DNA sequencing). In some embodiments, DNA or RNA may be assayed by one or more of: isothermal DNA/RNA amplification methods (e.g., loop-mediated isothermal amplification (LAMP), helicase dependent amplification (HDA), rolling circle amplification (RCA), recombinase polymerase amplification (RPA)), immunoassays, electrochemical assays, surface-enhanced Raman spectroscopy (SERS), quantum dot (QD)-based assays, molecular inversion probes, droplet digital PCR (ddPCR), CRISPR/Cas-based detection (e.g., CRISPR-typing PCR (ctPCR), specific high-sensitivity enzymatic reporter unlocking (SHERLOCK), DNA endonuclease targeted CRISPR trans reporter (DETECTR), and CRISPR-mediated analog multi-event recording apparatus (CAMERA)), and laser transmission spectroscopy (LTS).

The assay readouts may be quantified at one or more genomic loci (e.g., liver disease-associated genomic loci) to generate the data indicative of the liver disease state. For example, quantification of array hybridization or PCR corresponding to a plurality of genomic loci (e.g., liver disease-associated genomic loci) may generate data indicative of the liver disease state. Assay readouts may comprise quantitative PCR (qPCR) values, digital PCR (dPCR) values, digital droplet PCR (ddPCR) values, fluorescence values, etc., or normalized values thereof. The assay may be a home use test configured to be performed in a home setting.

In some embodiments, multiple assays are used to process cell-free biological samples of a subject. For example, a first assay may be used to process a first cell-free biological sample obtained or derived from the subject to generate a first dataset; and based at least in part on the first dataset, a second assay different from said first assay may be used to process a second cell-free biological sample obtained or derived from the subject to generate a second dataset indicative of the liver disease state. The first assay may be used to screen or process cell-free biological samples of a set of subjects, while the second or subsequent assays may be used to screen or process cell-free biological samples of a smaller subset of the set of subjects. The first assay may have a low cost and/or a high sensitivity of detecting one or more liver disease states (e.g., liver disease or condition), that is amenable to screening or processing cell-free biological samples of a relatively large set of subjects. The second assay may have a higher cost and/or a higher specificity of detecting one or more liver disease states, that is amenable to screening or processing cell-free biological samples of a relatively small set of subjects (e.g., a subset of the subjects screened using the first assay). The second assay may generate a second dataset having a specificity (e.g., for one or more liver disease states) greater than the first dataset generated using the first assay. As an example, one or more cell-free biological samples may be processed using a cfDNA assay on a large set of subjects and subsequently a metabolomics assay on a smaller subset of subjects, or vice versa. The smaller subset of subjects may be selected based at least in part on the results of the first assay.

Alternatively, multiple assays may be used to simultaneously process cell-free biological samples of a subject. For example, a first assay may be used to process a first cell-free biological sample obtained or derived from the subject to generate a first dataset indicative of the liver disease state; and a second assay different from the first assay may be used to process a second cell-free biological sample obtained or derived from the subject to generate a second dataset indicative of the liver disease state. Any or all of the first dataset and the second dataset may then be analyzed to assess the liver disease state of the subject. For example, a single diagnostic index or diagnosis score can be generated based on a combination of the first dataset and the second dataset. As another example, separate diagnostic indexes or diagnosis scores can be generated based on the first dataset and the second dataset.

The cell-free biological samples may be processed using a metabolomics assay. For example, a metabolomics assay can be used to identify a quantitative measure (e.g., indicative of a presence, absence, or relative amount) of each of a plurality of liver disease-associated metabolites in a cell-free biological sample of the subject. The metabolomics assay may be configured to process cell-free biological samples such as a blood sample (or derivatives thereof) of the subject. A quantitative measure (e.g., indicative of a presence, absence, or relative amount) of liver disease-associated metabolites in the cell-free biological sample may be indicative of one or more liver diseases. The metabolites in the cell-free biological sample may be produced (e.g., as an end product or a byproduct) as a result of one or more metabolic pathways corresponding to liver disease-associated genes. Assaying one or more metabolites of the cell-free biological sample may comprise isolating or extracting the metabolites from the cell-free biological sample. The metabolomics assay may be used to generate datasets indicative of the quantitative measure (e.g., indicative of a presence, absence, or relative amount) of each of a plurality of liver disease-associated metabolites in the cell-free biological sample of the subject.

The metabolomics assay may analyze a variety of metabolites in the cell-free biological sample, such as small molecules, lipids, amino acids, peptides, nucleotides, hormones and other signaling molecules, cytokines, minerals and elements, polyphenols, fatty acids, dicarboxylic acids, alcohols and polyols, alkanes and alkenes, keto acids, glycolipids, carbohydrates, hydroxy acids, purines, prostanoids, catecholamines, acyl phosphates, phospholipids, cyclic amines, amino ketones, nucleosides, glycerolipids, aromatic acids, retinoids, amino alcohols, pterins, steroids, carnitines, leukotrienes, indoles, porphyrins, sugar phosphates, coenzyme A derivatives, glucuronides, ketones, sugar phosphates, inorganic ions and gases, sphingolipids, bile acids, alcohol phosphates, amino acid phosphates, aldehydes, quinones, pyrimidines, pyridoxals, tricarboxylic acids, acyl glycines, cobalamin derivatives, lipoamides, biotin, and polyamines.

The metabolomics assay may comprise, for example, one or more of: mass spectroscopy (MS), targeted MS, gas chromatography (GC), high performance liquid chromatography (HPLC), capillary electrophoresis (CE), nuclear magnetic resonance (NMR) spectroscopy, ion-mobility spectrometry, Raman spectroscopy, electrochemical assay, or immune assay.

The cell-free biological samples may be processed using a methylation-specific assay. For example, a methylation-specific assay can be used to identify a quantitative measure (e.g., indicative of a presence, absence, or relative amount) of methylation each of a plurality of liver disease-associated genomic loci in a cell-free biological sample of the subject. Additionally, or alternatively, a methylation-specific assay can be used to identify a qualitative measure of methylation (e.g., a methylation pattern based on relative amount) of a plurality of liver disease-associated genomic loci in a cell-free biological sample of the subject. The methylation-specific assay may be configured to process cell-free biological samples such as a blood sample (or derivatives thereof) of the subject. A quantitative measure (e.g., indicative of a presence, absence, or relative amount) of methylation of liver disease-associated genomic loci in the cell-free biological sample may be indicative of one or more liver disease states. A qualitative measure of methylation (e.g., a methylation pattern based on relative amount) of liver disease-associated genomic loci in the cell-free biological sample may be indicative of one or more liver disease states. The methylation-specific assay may be used to generate datasets indicative of the quantitative measure and/or the qualitative measure of methylation of each of a plurality of liver disease-associated genomic loci in the cell-free biological sample of the subject.

The methylation-specific assay may comprise, for example, one or more of: a methylation-aware sequencing (e.g., using bisulfite treatment or bisulfite-free treatment), enzymatic methylation sequencing, methylation-specific PCR (MSP), methylation-sensitive restriction enzyme (MSRE) digestion, pyrosequencing, methylation-sensitive single-strand conformation analysis (MS-SSCA), high-resolution melting analysis (HRM), methylation-sensitive single-nucleotide primer extension (MS-SnuPE), base-specific cleavage/MALDI-TOF, microarray-based methylation assay, methylation-specific PCR, targeted bisulfite sequencing, oxidative bisulfite sequencing, mass spectroscopy-based bisulfite sequencing, or reduced representation bisulfite sequence (RRBS).

Bisulfite sequencing or treatment involves the treatment of DNA with bisulfite (e.g., sodium bisulfite) that converts cytosine residues to uracil residues, while 5-methylcytosine residues unaffected. As a result, DNA that has been treated with bisulfite may retain only methylated cytosines.

Targeted bisulfite sequencing includes hybridization in which pre-designed oligonucleotides may be used to probe or target particular genomic regions of interest, e.g., CpG islands, gene promoters, and other significant methylated regions (e.g., liver disease-associated genomic loci). Targeted bisulfite sequencing may include an amplification to amplify multiple bisulfite-converted DNA regions in a single reaction. Specific primers may be designed to capture regions of interest and evaluate site-specific DNA methylation patterns.

Pyrosequencing is a sequencing-by-synthesis method that quantitatively monitors the real-time incorporation of nucleotides through the enzymatic conversion of released pyrophosphate into a proportional light signal. Analysis of DNA methylation patterns by pyrosequencing may combine a simple reaction protocol with reproducible and accurate measures of the degree of methylation at several CpGs in close proximity with high quantitative resolution. After bisulfite treatment and PCR amplification, the degree of each methylation at each CpG position in a sequence may be determined from the ratio of T and C. The process of purification and sequencing can be repeated for the same template to analyze other CpGs in the same amplification product.

RRBS is an efficient, high-throughput technique for analyzing the genome-wide methylation profiles on a single nucleotide level. RRBS may combine restriction enzymes and bisulfite sequencing to enrich for areas of the genome with a high CpG content. RRBS can reduce the amount of nucleotides required to sequence to 1% of the genome. The fragments that comprise the reduced genome may still include the majority of promoters, as well as regions such as repeated sequences that are difficult to profile using conventional bisulfite sequencing approaches.

In some cases, bisulfite conversion methods may be lead to damage of sample DNA, resulting in fragmentation, loss, and bias, thereby limiting usefulness. Bisulfite-free methylation sequencing methods allow conversion of methylated cytosines while minimizing these shortcomings. For example, bisulfite-free methylation sequencing of cfDNA may be advantageous as cfDNA may be present at very low concentrations in plasma and may be a limiting resource in liquid biopsy applications.

Enzymatic methylation sequencing provides a bisulfite-free approach that minimizes damage of sample DNA for methylation detection. Such enzymatic approaches may provide greater mapping efficiency, more uniform GC coverage, detection of more CpGs with fewer sequence reads, and more uniform dinucleotide distribution. Enzymatic methylation sequencing methods may include treatment with a methylcytosine dioxygenase, such as ten-eleven translocation (TET) enzyme; a glucosyltransferase, such as β-glucosyltransferase (BGT); and/or a cytidine deaminase, such as activation-induced (cytidine) deaminase (AID) and apolipoprotein B mRNA editing enzyme, catalytic polypeptide (APOBEC).

Methylcytosine dioxygenases may be used to convert 5mC and 5hmC residues to 5caC to protect these methylated residues from deamination in downstream processing operations. Non-limiting examples of methylcytosine dioxygenases include, TET1, TET2, TET3, and catalytically active variants or fusion proteins thereof. Glucosyltransferases may be used to add a glucosyl group to 5hmC also to protect these methylated residues from downstream deamination. Cytidine deaminases may be used to deaminate 5mC residues to uracil and 5hmC residues to thymine. Non-limiting examples of cytidine deaminases include APOBEC3A and catalytically active variants or fusion proteins thereof. Combinations of one of more enzymes may be used for bisulfite-free methylation sequencing.

TET-assisted pyridine borane sequencing (TAPS) uses a TET enzyme to oxidize 5mC and 5hmC residues to 5caC. Pyridine borane is then used to reduce 5caC to dihydrouracil, which is then converted to thymine after amplification. TAPS may be performed in two other ways: TAPSβ and chemical-assisted pyridine borane sequencing (CAPS). In TAPSB, β-glucosyltransferase is used to label 5hmC with glucose to protect 5hmC from the oxidation and reduction reactions, allowing for specific detection of 5mC. In CAPS, potassium perruthenate acts as the chemical replacement for TET and specifically oxidizes 5hmC, thus allowing for direct detection of 5hmC.

Methylation-specific PCR (MSP) is a qualitative DNA methylation analysis. MSP may have advantages such as ease of design and execution, sensitivity in the ability to detect small quantities of methylated DNA, and the ability to rapidly screen a large number of samples without expensive laboratory equipment. This assay may require modification of the genomic DNA by sodium bisulfite and two independent primer sets for PCR amplification, one pair designed to recognize the methylated versions of the bisulfite-modified sequence and the other pair designed to recognize the unmethylated versions of the bisulfite-modified sequence. The amplicons may be visualized using ethidium bromide staining following agarose gel electrophoresis. Amplicons of the expected size produced from either primer pair may be indicative of the presence of DNA in the original sample with the respective methylation status.

In some embodiments, methylation-sensitive restriction enzyme (MSRE) digestion may be used to analyze methylation status of cytosine residues in CpG sequences. The enzymes may be unable to cleave methylated-cytosine residues, leaving methylated DNA fragments intact. Sample DNA obtained or derived from a subject can be digested with one or more MSREs. For example, liver disease-associated genomic loci described herein may contain at least one specific MSRE recognized sequence (recognition site). The sample DNA may be cut (digested) based on to its methylation level in which higher methylation results in a lesser degree of digestion by the enzyme. For example, if a DNA sample from a healthy subject is less methylated than another DNA sample from a liver disease patient for the CpGs on the recognition sequence, the DNA may be cut more extensively.

For example, DNA molecules may be extracted from the biological sample. A first portion of the extracted DNA molecules may be subjected to CpG site fragmentation conditions, such as MSREs digestion, while a second portion of the extracted DNA molecules may not be subjected to such fragmentation conditions. Next, qPCR amplification of at least one biomarker locus, an internal control locus, may be performed (e.g., using qPCR primers). Cycle threshold (Ct) values may be obtained for each amplified region of a set of genomic regions (e.g., liver disease-associated biomarkers) and normalized based on the internal control locus. A qPCR signal intensity may be calculated for the biomarker locus, where the signal intensity=2\hat{ }[Ct, biomarker restriction locus-Ct, internal control locus]. A probability score may then be calculated, which reflects the correlation between the biomarker signal intensity in the subject and “disease” references and/or the correlation between the biomarker signal intensity in the subject and “healthy” references.

In some embodiments, a control locus may be designed to exclude MSRE restriction sites. In some embodiments, a fixed proportion of control DNA is added into the sample DNA for all test subjects. In some embodiments, at least one pair of qPCR primers is designed for each target genomic region of a biomarker. For each patient, two qPCR reactions are run independently on the same qPCR target: a first qPCR reaction is run on a first portion of the sample DNA that contains MSRE-digested DNA template, and a second qPCR reaction is run on a second portion of the sample DNA that contains undigested DNA templates. The undigested template may be used to represent the fully methylated DNA. After the purification of the MSRE digestion, the same amount of DNA may be used for the digested and undigested templates. The signal intensity of the qPCR reaction may be generated from the cycle threshold (Ct) values. The Ct value refers to the number of cycles required for a fluorescent signal to cross a given cycle threshold (e.g., at which the signal exceeds a background level). Ct levels may be inversely proportional to the amount of target nucleic acid in a sample (e.g., the lower the Ct level of a given sample, the greater the amount of target nucleic acid in the sample). For each locus of a given sample, the Ct difference (delta Ct) between the first qPCR reaction (run on the digested DNA template) and the second qPCR reaction (run on the undigested DNA template) may be calculated and used to indicate the DNA methylation level of the sample. Thus, the delta Ct value can represent the subject's DNA methylation level for the target region. For example, the undigested DNA may have low Ct values, while the digested DNA from a normal individual may have high Ct values, thereby resulting in large absolute delta Ct values. Otherwise, the delta Ct values from a subject having liver disease may be small (e.g., close to 0).

The cell-free biological samples may be processed using a proteomics assay. For example, a proteomics assay can be used to identify a quantitative measure (e.g., indicative of a presence, absence, or relative amount) of each of a plurality of liver disease-associated proteins or polypeptides in a cell-free biological sample of the subject. The proteomics assay may be configured to process cell-free biological samples such as a blood sample (or derivatives thereof) of the subject. A quantitative measure (e.g., indicative of a presence, absence, or relative amount) of liver disease-associated proteins or polypeptides in the cell-free biological sample may be indicative of one or more liver disease states. The proteins or polypeptides in the cell-free biological sample may be produced (e.g., as an end product or a byproduct) as a result of one or more biochemical pathways corresponding to liver disease-associated genes. Assaying one or more proteins or polypeptides of the cell-free biological sample may comprise isolating or extracting the proteins or polypeptides from the cell-free biological sample. The proteomics assay may be used to generate datasets indicative of the quantitative measure (e.g., indicative of a presence, absence, or relative amount) of each of a plurality of liver disease-associated proteins or polypeptides in the cell-free biological sample of the subject.

The proteomics assay may analyze a variety of proteins or polypeptides in the cell-free biological sample, such as proteins made under different cellular conditions (e.g., development, cellular differentiation, or cell cycle). The proteomics assay may comprise, for example, one or more of: an antibody-based immunoassay, an Edman degradation assay, a mass spectrometry-based assay (e.g., matrix-assisted laser desorption/ionization (MALDI) and electrospray ionization (ESI)), a top-down proteomics assay, a bottom-up proteomics assay, a mass spectrometric immunoassay (MSIA), a stable isotope standard capture with anti-peptide antibodies (SISCAPA) assay, a fluorescence two-dimensional differential gel electrophoresis (2-D DIGE) assay, a quantitative proteomics assay, a protein microarray assay, or a reverse-phased protein microarray assay. The proteomics assay may detect post-translational modifications of proteins or polypeptides (e.g., phosphorylation, ubiquitination, methylation, acetylation, glycosylation, oxidation, and nitrosylation). The proteomics assay may identify or quantify one or more proteins or polypeptides from a database (e.g., Human Protein Atlas, PeptideAtlas, and UniProt).

### Kits

The present disclosure provides kits for identifying or monitoring a liver disease state of a subject. A kit may comprise probes for identifying a quantitative measure (e.g., indicative of a presence, absence, or relative amount) of sequences at each of a plurality of liver disease-associated genomic loci in a cell-free biological sample of the subject. A quantitative measure (e.g., indicative of a presence, absence, or relative amount) of sequences at each of a plurality of liver disease-associated genomic loci in the cell-free biological sample may be indicative of one or more liver disease states. The probes may be selective for the sequences at the plurality of liver disease-associated genomic loci in the cell-free biological sample. A kit may comprise instructions for using the probes to process the cell-free biological sample to generate datasets indicative of a quantitative measure (e.g., indicative of a presence, absence, or relative amount) of sequences at each of the plurality of liver disease-associated genomic loci in a cell-free biological sample of the subject.

The probes in the kit may be selective for the sequences at the plurality of liver disease-associated genomic loci in the cell-free biological sample. The probes in the kit may be configured to selectively enrich nucleic acid molecules (e.g., RNA or DNA) corresponding to the plurality of liver disease-associated genomic loci. The probes in the kit may be nucleic acid primers. The probes in the kit may have sequence complementarity with nucleic acid sequences from one or more of the plurality of liver disease-associated genomic loci or genomic regions. The plurality of liver disease-associated genomic loci or genomic regions may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, at least 90, at least 95, at least 100, at least 200, at least 300, at least 400, at least 500, at least 600, at least 700, at least 800, at least 900, at least 1000, or more distinct liver disease-associated genomic loci or genomic regions. The plurality of liver disease-associated genomic loci or genomic regions may comprise one or more members selected from the group consisting of genes listed in TABLE 1.

The instructions in the kit may comprise instructions to assay the cell-free biological sample using the probes that are selective for the sequences at the plurality of liver disease-associated genomic loci in the cell-free biological sample. These probes may be nucleic acid molecules (e.g., RNA or DNA) having sequence complementarity with nucleic acid sequences (e.g., RNA or DNA) from one or more of the plurality of liver disease-associated genomic loci. These nucleic acid molecules may be primers or enrichment sequences. The instructions to assay the cell-free biological sample may comprise introductions to perform array hybridization, PCR, or nucleic acid sequencing to process the cell-free biological sample to generate datasets indicative of a quantitative measure (e.g., indicative of a presence, absence, or relative amount) of sequences at each of the plurality of liver disease-associated genomic loci in the cell-free biological sample. A quantitative measure (e.g., indicative of a presence, absence, or relative amount) of sequences at each of a plurality of liver disease-associated genomic loci in the cell-free biological sample may be indicative of one or more liver disease states.

The instructions in the kit may comprise instructions to measure and interpret assay readouts, which may be quantified at one or more of the plurality of liver disease-associated genomic loci to generate the datasets indicative of a quantitative measure (e.g., indicative of a presence, absence, or relative amount) of sequences at each of the plurality of liver disease-associated genomic loci in the cell-free biological sample. For example, quantification of array hybridization or polymerase chain reaction (PCR) corresponding to the plurality of liver disease-associated genomic loci may generate the datasets indicative of a quantitative measure (e.g., indicative of a presence, absence, or relative amount) of sequences at each of the plurality of liver disease-associated genomic loci in the cell-free biological sample. Assay readouts may comprise quantitative PCR (qPCR) values, digital PCR (dPCR) values, digital droplet PCR (ddPCR) values, fluorescence values, etc., or normalized values thereof.

A kit may comprise a metabolomics assay for identifying a quantitative measure (e.g., indicative of a presence, absence, or relative amount) of each of a plurality of liver disease-associated metabolites in a cell-free biological sample of the subject. A quantitative measure (e.g., indicative of a presence, absence, or relative amount) of liver disease-associated metabolites in the cell-free biological sample may be indicative of one or more liver disease states. The metabolites in the cell-free biological sample may be produced (e.g., as an end product or a byproduct) as a result of one or more metabolic pathways corresponding to liver disease-associated genes. A kit may comprise instructions for isolating or extracting the metabolites from the cell-free biological sample and/or for using the metabolomics assay to generate datasets indicative of the quantitative measure (e.g., indicative of a presence, absence, or relative amount) of each of a plurality of liver disease-associated metabolites in the cell-free biological sample of the subject.

### Machine Learning Models

After using one or more assays to process one or more cell-free biological samples derived from the subject to generate one or more datasets indicative of the liver disease or condition, a trained algorithm may be used to process one or more of the datasets (e.g., at each of a plurality of liver disease-associated genomic loci) to determine the liver disease state. For example, the trained algorithm may be used to determine quantitative measures of sequences at each of the plurality of liver disease-associated genomic loci in the cell-free biological samples. The trained algorithm may be configured to identify the liver disease state with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99% for at least about 25, at least about 50, at least about 100, at least about 150, at least about 200, at least about 250, at least about 300, at least about 350, at least about 400, at least about 450, at least about 500, or more than about 500 independent samples.

The trained algorithm may comprise a supervised machine learning algorithm. The trained algorithm may comprise a classification and regression tree (CART) algorithm. The supervised machine learning algorithm may comprise a classifier or a regression. The supervised machine learning algorithm may comprise, for example, a deep learning algorithm, a support vector machine (SVM), a neural network, a random forest, a linear regression, or a logistic regression. The trained algorithm may comprise an unsupervised machine learning algorithm.

The trained algorithm may be configured to accept a plurality of input variables and to produce one or more output values based on the plurality of input variables. The plurality of input variables may comprise one or more datasets indicative of a liver disease state. For example, an input variable may comprise a number of sequences corresponding to or aligning to each of the plurality of liver disease-associated genomic loci. The plurality of input variables may also include clinical health data of a subject.

The trained algorithm may comprise a classifier, such that each of the one or more output values comprises one of a fixed number of possible values (e.g., a linear classifier, a logistic regression classifier, etc.) indicating a classification of the cell-free biological sample by the classifier. The trained algorithm may comprise a binary classifier, such that each of the one or more output values comprises one of two values (e.g., {0, 1}, {positive, negative}, or {high-risk, low-risk}) indicating a classification of the cell-free biological sample by the classifier. The trained algorithm may be another type of classifier, such that each of the one or more output values comprises one of more than two values (e.g., {0, 1, 2}, {positive, negative, or indeterminate}, or {high-risk, intermediate-risk, or low-risk}) indicating a classification of the cell-free biological sample by the classifier. The output values may comprise descriptive labels, numerical values, or a combination thereof. Some of the output values may comprise descriptive labels. Such descriptive labels may provide an identification or indication of the liver disease or disorder state of the subject. Such descriptive labels may comprise, for example, positive, negative, high-risk, intermediate-risk, low-risk, or indeterminate. Such descriptive labels may provide an identification of a treatment for the subject's liver disease state, and may comprise, for example, a therapeutic intervention (e.g., vitamin E supplementation, a weight loss agent, an anti-hypertensive agent, an anti-diabetic agent, a cholesterol-lowering agent, an exercise regiment, a diet regimen, bariatric surgery, a GLP1 (glucagon-like peptide-1) receptor agonist, a FGF (fibroblast growth factor) analog, a THR (thyroid hormone receptor) agonist, a SCD-1 (stearoyl-coenzyme A desaturase 1) inhibitor, a FAS (fatty acid synthase) inhibitor, a FXR (farnesoid X receptor) agonist, an ACC (acetyl-CoA carboxylase) inhibitor, a PPAR (peroxisome proliferator-activated receptor) agonist, a targeted genetic modifier (including, e.g., PNPLA3 or HSD17B13), a LOXL2 (lysyl oxidase-like 2) inhibitor, a pan-cyclophilin inhibitor, a pan-caspase inhibitor, a chemokine receptor (e.g., CCR2/CCR5) inhibitor, a galactin-3 inhibitor, a mitochondrial uncoupler or uncoupling agent, a structurally engineered fatty acid, or any combination thereof, a duration of the therapeutic intervention, and/or a dosage of the therapeutic intervention suitable to treat a liver disease condition. Such descriptive labels may provide an identification of secondary clinical tests that may be appropriate to perform on the subject, and may comprise, for example, a blood test, a liver biopsy, an imaging test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof. For example, such descriptive labels may provide a prognosis of the liver disease state of the subject. As another example, such descriptive labels may provide a relative assessment of the liver disease state (e.g., presence or absence, stage, or subtype) of the subject. Some descriptive labels may be mapped to numerical values, for example, by mapping “positive” to 1 And “negative” to 0.

Some of the output values may comprise numerical values, such as binary, integer, or continuous values. Such binary output values may comprise, for example, {0, 1}, {positive, negative}, or {high-risk, low-risk}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prognosis of the liver disease state of the subject. Some numerical values may be mapped to descriptive labels, for example, by mapping 1 to “positive” and 0 to “negative.”

Some of the output values may be assigned based on one or more cutoff values. For example, a binary classification of samples may assign an output value of “positive” or 1 if the sample indicates that the subject has at least a 50% probability of having a liver disease state. For example, a binary classification of samples may assign an output value of “negative” or 0 if the sample indicates that the subject has less than a 50% probability of having a liver disease state. In this case, a single cutoff value of 50% is used to classify samples into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of samples may assign an output value of “positive” or 1 if the sample indicates that the subject has a probability of having a liver disease of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of samples may assign an output value of “positive” or 1 if the sample indicates that the subject has a probability of having a liver disease state of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of samples may assign an output value of “negative” or 0 if the sample indicates that the subject has a probability of having a liver disease of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%. The classification of samples may assign an output value of “negative” or 0 if the sample indicates that the subject has a probability of having a liver disease state of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%.

The classification of samples may assign an output value of “indeterminate” or 2 if the sample is not classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent samples. Each of the independent samples may comprise a cell-free biological sample from a subject, associated datasets obtained by assaying the cell-free biological sample (as described herein), and one or more known output values corresponding to the cell-free biological sample (e.g., a clinical diagnosis, prognosis, absence, or treatment efficacy of a liver disease state of the subject). Independent samples may comprise cell-free biological samples and associated datasets and outputs obtained or derived from a plurality of different subjects. Independent samples may comprise cell-free biological samples and associated datasets and outputs obtained at a plurality of different time points from the same subject (e.g., on a regular basis such as weekly, biweekly, or monthly). Independent samples may be associated with presence of the liver disease state (e.g., training samples comprising cell-free biological samples and associated datasets and outputs obtained or derived from a plurality of subjects known to have the liver disease state). Independent samples may be associated with absence of the liver disease state (e.g., training samples comprising cell-free biological samples and associated datasets and outputs obtained or derived from a plurality of subjects who are known to not have a previous diagnosis of the liver disease state or who have received a negative test result for the liver disease state).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent samples. The independent samples may comprise cell-free biological samples associated with presence of the liver disease state and/or cell-free biological samples associated with absence of the liver disease state. The trained algorithm may be trained with no more than about 500, no more than about 450, no more than about 400, no more than about 350, no more than about 300, no more than about 250, no more than about 200, no more than about 150, no more than about 100, or no more than about 50 independent samples associated with presence of the liver disease. In some embodiments, the cell-free biological sample is independent of samples used to train the trained algorithm.

The trained algorithm may be trained with a first number of independent samples associated with presence of the liver disease and a second number of independent samples associated with absence of the liver disease. The first number of independent samples associated with presence of the liver disease may be no more than the second number of independent samples associated with absence of the liver disease. The first number of independent samples associated with presence of the liver disease may be equal to the second number of independent samples associated with absence of the liver disease state. The first number of independent samples associated with presence of the liver disease state may be greater than the second number of independent samples associated with absence of the liver disease state.

The trained algorithm may be configured to identify the liver disease at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent samples. The accuracy of identifying the liver disease state by the trained algorithm may be calculated as the percentage of independent samples (e.g., subjects known to have the liver disease state or subjects with negative clinical test results for the liver disease state) that are correctly identified or classified as having or not having the liver disease state.

The trained algorithm may be configured to identify the liver disease state with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the liver disease state using the trained algorithm may be calculated as the percentage of cell-free biological samples identified or classified as having the liver disease state that correspond to subjects that truly have the liver disease state.

The trained algorithm may be configured to identify the liver disease state with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the liver disease state using the trained algorithm may be calculated as the percentage of cell-free biological samples identified or classified as not having the liver disease state that correspond to subjects that truly do not have the liver disease state.

The trained algorithm may be configured to identify the liver disease state with a clinical sensitivity at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the liver disease state using the trained algorithm may be calculated as the percentage of independent samples associated with presence of the liver disease state (e.g., subjects known to have the liver disease state) that are correctly identified or classified as having the liver disease state.

The trained algorithm may be configured to identify the liver disease state with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the liver disease state using the trained algorithm may be calculated as the percentage of independent samples associated with absence of the liver disease state (e.g., subjects with negative clinical test results for the liver disease state) that are correctly identified or classified as not having the liver disease state.

The trained algorithm may be configured to identify the liver disease state with an area Under Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or more. The AUC may be calculated as an integral of the receiver operator characteristic (ROC) curve, e.g., the area under the ROC curve (AUROC), associated with the trained algorithm in classifying cell-free biological samples as having or not having the liver disease state.

The trained algorithm may be adjusted or tuned to improve one or more of the performance, accuracy, PPV, NPV, clinical sensitivity, clinical specificity, or AUC of identifying the liver disease state. The trained algorithm may be adjusted or tuned by adjusting parameters of the trained algorithm (e.g., a set of cutoff values used to classify a cell-free biological sample as described elsewhere herein, or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of liver disease-associated genomic loci may be identified as most influential or most important to be included for making high-quality classifications or identifications of liver disease (or sub-types of liver disease). The plurality of liver disease-associated genomic loci or a subset thereof may be ranked based on classification metrics indicative of each genomic locus's influence or importance toward making high-quality classifications or identifications of liver disease (or sub-types of liver disease). Such metrics may be used to reduce, in some cases significantly, the number of input variables (e.g., predictor variables) that may be used to train the trained algorithm to a desired performance level (e.g., based on a desired minimum accuracy, PPV, NPV, clinical sensitivity, clinical specificity, AUC, positive likelihood ratio, negative likelihood ratio, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a predetermined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

The accuracy of a trained algorithm may be context-dependent. In some cases, the accuracy may be based on training samples from a general population. In other cases, the accuracy may be based on training samples from a high risk population, e.g., a population suspected to have the liver disease. Several factors may be considered for interpreting test performance of a trained algorithm, including: 1) prevalence of the disease or condition, e.g., how many people in a target population having the disease; and 2) whether the test is for diagnosing the disease, i.e., a positive (rule in) test, or whether the test is for confirming a subject is disease free, i.e., negative (rule out) test.

On the other hand, metrics such as pre-test/post-test probability, Bayes factor, likelihood ratio, or information gain may be context independent. These metrics measure the amount of new information provided by a test. For example, the pre-test and post-test probability ratio may be calculated by “the probability of a subject in a target population having a condition” divided by “the probability of a subject in the target population with a given test result having the condition”. As an example, about 5% of the U.S. population have NASH; thus, the pre-test probability of NASH in the U.S. population is 5%. If 50% of the subject that a test detects actually have NASH, then the post-test probability is 50% and the pre-test/post-test ratio is 10. As another example, if about 40% of subjects in a high-risk population have NASH and a hypothetical test is performed on this high-risk population, 50% of people detected by the test truly have NASH and the pre-test/post-test ratio is 1.25.

### Identifying or Monitoring a Liver Disease State

After using a trained algorithm to process the dataset, the liver disease state may be identified or monitored in the subject. The identification may be based at least in part on quantitative measures of sequence reads of the dataset at a panel of liver disease-associated genomic loci (e.g., DNA at the liver disease-associated genomic loci or quantitative measures of RNA transcripts), proteomic data comprising quantitative measures of proteins of the dataset at a panel of liver disease-associated proteins, and/or metabolome data comprising quantitative measures of a panel of liver disease-associated metabolites.

The liver disease state may be identified in the subject at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The accuracy of identifying the liver disease state by the trained algorithm may be calculated as the percentage of independent samples (e.g., subjects known to have the liver disease state or subjects with negative clinical test results for the liver disease state) that are correctly identified or classified as having or not having the liver disease state.

The liver disease state may be identified in the subject with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of identifying the liver disease state using the trained algorithm may be calculated as the percentage of cell-free biological samples identified or classified as having the liver disease state that correspond to subjects that truly have the liver disease state.

The liver disease state may be identified in the subject with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of identifying the liver disease state using the trained algorithm may be calculated as the percentage of cell-free biological samples identified or classified as not having the liver disease state that correspond to subjects that truly do not have the liver disease state.

The liver disease state may be identified in the subject with a clinical sensitivity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical sensitivity of identifying the liver disease state using the trained algorithm may be calculated as the percentage of independent samples associated with presence of the liver disease state (e.g., subjects known to have the liver disease state) that are correctly identified or classified as having the liver disease state.

The liver disease state may be identified in the subject with a clinical specificity of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more. The clinical specificity of identifying the liver disease state using the trained algorithm may be calculated as the percentage of independent samples associated with absence of the liver disease state (e.g., subjects with negative clinical test results for the liver disease state) that are correctly identified or classified as not having the liver disease state.

Likelihood ratio may be used for assessing the performance of a diagnostic test. The liver disease state may be identified or ruled out in the subject based on a likelihood ratio, e.g., a positive likelihood ratio or a negative likelihood ratio. A likelihood ratio may be independent of the prevalence of disease in the training population, and thus, more representative of prevalence of the disease in a target population. Because a likelihood ratio is independent of disease prevalence, a likelihood ratio may be more directly related to the performance of a given diagnostic test.

A positive likelihood ratio may be calculated as sensitivity/(1-specificity). The liver disease state may be identified in the subject with a positive likelihood ratio of at least about 1, at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, or at least about 1000.

A negative likelihood ratio may be calculated as (1-sensitivity)/specificity. The liver disease state may be ruled out in the subject with a negative likelihood ratio of at most about 1, at most about 0.99, at most about 0.95, at most about 0.9, at most about 0.8, at most about 0.7, at most about 0.75, at most about 0.6 at most about 0.5, at most about 0.4, at most about 0.3, at most about 0.25, at most about 0.2, at most about 0.1, at most about 0.09, at most about 0.08, at most about 0.07, at most about 0.06, at most about 0.05, at most about 0.04, at most about 0.03, at most about 0.02, at most about 0.01, at most about 0.009, at most about 0.008, at most about 0.007, at most about 0.006, at most about 0.005, at most about 0.004, at most about 0.003, at most about 0.002, or at most about 0.001.

In an aspect, the present disclosure provides a method for determining that a subject is at risk of developing a liver disease, comprising assaying a cell-free biological sample derived from the subject to generate a dataset that is indicative of the risk of developing the liver disease at a specificity of at least 80%, and using a trained algorithm that is trained on samples independent of the cell-free biological sample to determine that the subject is at risk of developing the liver disease at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

After the liver disease is identified in a subject, a sub-type of the liver disease (e.g., selected from among a plurality of sub-types of the liver disease) may further be identified. The sub-type of the liver disease may be determined based at least in part on the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites. For example, the subject may be identified as being at risk of a sub-type of a liver disease (e.g., selected from among a plurality of sub-types of a liver disease). After identifying the subject as being at risk of a sub-type of a liver disease, a clinical intervention for the subject may be selected based at least in part on the sub-type of liver disease for which the subject is identified as being at risk. In some embodiments, the clinical intervention is selected from a plurality of clinical interventions (e.g., clinically indicated for different sub-types of a liver disease).

In some embodiments, the trained algorithm may determine that the subject is at risk of a liver disease of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more.

The trained algorithm may determine that the subject is at risk of a liver disease at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.99%, at least about 99.999%, or more.

Upon identifying the subject as having the liver disease state, the subject may be optionally provided with a therapeutic intervention (e.g., prescribing an appropriate course of treatment to treat the liver disease state of the subject). The therapeutic intervention may comprise a prescription of an effective dose of a drug, a further testing or evaluation of the liver disease state, a further monitoring of the liver disease state, an exercise regimen, a diet regimen, bariatric surgery, or a combination thereof. The therapeutic intervention may comprise vitamin E supplementation, a weight loss agent, an anti-hypertensive agent, an anti-diabetic agent, a cholesterol-lowering agent, an exercise regiment, a diet regimen, bariatric surgery, a GLP1 (glucagon-like peptide-1) receptor agonist, a FGF (fibroblast growth factor) analog, a THR (thyroid hormone receptor) agonist, a SCD-1 (stearoyl-coenzyme A desaturase 1) inhibitor, a FAS (fatty acid synthase) inhibitor, a FXR (farnesoid X receptor) agonist, an ACC (acetyl-CoA carboxylase) inhibitor, a PPAR (peroxisome proliferator-activated receptor) agonist, a targeted genetic modifier (including, e.g., PNPLA3 or HSD17B13), a LOXL2 (lysyl oxidase-like 2) inhibitor, a pan-cyclophilin inhibitor, a pan-caspase inhibitor, a chemokine receptor (e.g., CCR2/CCR5) inhibitor, a galactin-3 inhibitor, a mitochondrial uncoupler or uncoupling agent, a structurally engineered fatty acid, or a combination thereof. If the subject is currently being treated for the liver disease state with a course of treatment, the therapeutic intervention may comprise a subsequent different course of treatment (e.g., to increase treatment efficacy due to non-efficacy of the current course of treatment).

The therapeutic intervention may comprise recommending the subject for a secondary clinical test to confirm a diagnosis of the liver disease state. This secondary clinical test may comprise a blood test, a liver biopsy, an imaging test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof.

Upon identifying the subject as having the liver disease state, the subject may be optionally determined as being ineligible for liver disease transplant. Upon identifying the subject as not having the liver disease state, the subject may be optionally determined as being eligible for liver disease transplant. A subject may be determined as being eligible as the liver transplant donor if the subject is not identified as having or being at the increased risk of developing the liver disease. A subject may be determined as being eligible as the liver transplant recipient if the subject is identified as having or being at the increased risk of developing the liver disease.

Various therapeutic interventions and clinical tests for liver disease may be used in combination with the methods described herein. For example, a therapeutic intervention may be administered to a subject upon determining that the subject has a liver disease. As another example, a prophylactic intervention may be administered to a subject upon determining that the subject has an elevated risk of having a liver disease. Example liver disease interventions and clinical tests are described in Vittal et al. Clin Liver Dis. 2019 August; 23(3): 417-432; Marroni et al. World J Gastroenterol. 2018 Jul. 14; 24(26): 2785-2805; Leoni et al. World J Gastroenterol. 2018 Aug. 14; 24(30): 3361-3373; and Sumida et al. J Gastroenterol. 2018 March; 53(3): 362-376, each of which is incorporated herein by reference in its entirety.

The quantitative measures of sequence reads of the dataset at the panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites may be assessed over a duration of time to monitor a patient (e.g., subject who has a liver disease or who is being treated for a liver disease). In such cases, the quantitative measures of the dataset of the patient may change during the course of treatment. For example, the quantitative measures of the dataset of a patient with decreasing risk of the liver disease due to an effective treatment may shift toward the profile or distribution of a healthy subject (e.g., a subject without a liver disease or condition). Conversely, for example, the quantitative measures of the dataset of a patient with increasing risk of the liver disease due to an ineffective treatment may shift toward the profile or distribution of a subject with higher risk of the liver disease or a more advanced liver disease.

The liver disease of the subject may be monitored by monitoring a course of treatment for treating the liver disease of the subject. The monitoring may comprise assessing the liver disease state of the subject at two or more time points. The assessing may be based at least on the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites determined at each of the two or more time points.

In some embodiments, a difference in the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites determined between the two or more time points may be indicative of one or more clinical indications, such as (i) a diagnosis of the liver disease of the subject, (ii) a prognosis of the liver disease of the subject, (iii) an increased risk of the liver disease of the subject, (iv) a decreased risk of the liver disease of the subject, (v) an efficacy of the course of treatment for treating the liver disease of the subject, and (vi) a non-efficacy of the course of treatment for treating the liver disease of the subject.

In some embodiments, a difference in the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites determined between the two or more time points may be indicative of a diagnosis of the liver disease of the subject. For example, if the liver disease was not detected in the subject at an earlier time point but was detected in the subject at a later time point, then the difference is indicative of a diagnosis of the liver disease of the subject. A clinical action or decision may be made based on this indication of diagnosis of the liver disease of the subject, such as, for example, prescribing a new therapeutic intervention for the subject. The clinical action or decision may comprise recommending the subject for a secondary clinical test to confirm the diagnosis of the liver disease state. This secondary clinical test may comprise a blood test, a liver biopsy, an imaging test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof.

In some embodiments, a difference in the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites determined between the two or more time points may be indicative of a prognosis of the liver disease state of the subject.

In some embodiments, a difference in the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites determined between the two or more time points may be indicative of the subject having an increased risk of the liver disease state. For example, if the liver disease state was detected in the subject both at an earlier time point and at a later time point, and if the difference is a positive difference (e.g., the quantitative measures of sequence reads of the dataset at a panel of liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites increased from the earlier time point to the later time point), then the difference may be indicative of the subject having an increased risk of the liver disease state. A clinical action or decision may be made based on this indication of the increased risk of the liver disease state, e.g., prescribing a new therapeutic intervention or switching therapeutic interventions (e.g., ending a current treatment and prescribing a new treatment) for the subject. The clinical action or decision may comprise recommending the subject for a secondary clinical test to confirm the increased risk of the liver disease state. This secondary clinical test may comprise a blood test, a liver biopsy, an imaging test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof.

In some embodiments, a difference in the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites determined between the two or more time points may be indicative of the subject having a decreased risk of the liver disease state. For example, if the liver disease was detected in the subject both at an earlier time point and at a later time point, and if the difference is a negative difference (e.g., the quantitative measures of sequence reads of the dataset at a panel of liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites decreased from the earlier time point to the later time point), then the difference may be indicative of the subject having a decreased risk of the liver disease state. A clinical action or decision may be made based on this indication of the decreased risk of the liver disease state (e.g., continuing or ending a current therapeutic intervention) for the subject. The clinical action or decision may comprise recommending the subject for a secondary clinical test to confirm the decreased risk of the liver disease state. This secondary clinical test may comprise a blood test, a liver biopsy, an imaging test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof.

In some embodiments, a difference in the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites determined between the two or more time points may be indicative of an efficacy of the course of treatment for treating the liver disease state of the subject. For example, if the liver disease was detected in the subject at an earlier time point but was not detected in the subject at a later time point, then the difference may be indicative of an efficacy of the course of treatment for treating the liver disease of the subject. A clinical action or decision may be made based on this indication of the efficacy of the course of treatment for treating the liver disease of the subject, e.g., continuing or ending a current therapeutic intervention for the subject. The clinical action or decision may comprise recommending the subject for a secondary clinical test to confirm the efficacy of the course of treatment for treating the liver disease state. This secondary clinical test may comprise a blood test, a liver biopsy, an imaging test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof.

In some embodiments, a difference in the quantitative measures of sequence reads of the dataset at a panel of liver disease-associated genomic loci (e.g., quantitative measures of DNA at the liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites determined between the two or more time points may be indicative of a non-efficacy of the course of treatment for treating the liver disease state of the subject. For example, if the liver disease state was detected in the subject both at an earlier time point and at a later time point, and if the difference is a positive or zero difference (e.g., the quantitative measures of sequence reads of the dataset at a panel of liver disease-associated genomic loci or RNA transcripts), proteomic data comprising quantitative measures of proteins of the dataset at a panel of liver disease-associated proteins, and/or metabolome data comprising quantitative measures of a panel of liver disease-associated metabolites increased or remained at a constant level from the earlier time point to the later time point), and if an efficacious treatment was indicated at an earlier time point, then the difference may be indicative of a non-efficacy of the course of treatment for treating the liver disease of the subject. A clinical action or decision may be made based on this indication of the non-efficacy of the course of treatment for treating the liver disease of the subject, e.g., ending a current therapeutic intervention and/or switching to (e.g., prescribing) a different new therapeutic intervention for the subject. The clinical action or decision may comprise recommending the subject for a secondary clinical test to confirm the non-efficacy of the course of treatment for treating the liver disease. This secondary clinical test may comprise a blood test, a liver biopsy, an imaging test, a computed tomography (CT) scan, a magnetic resonance imaging (MRI) scan, an ultrasound scan, a chest X-ray, a positron emission tomography (PET) scan, a PET-CT scan, a cell-free biological cytology, or any combination thereof.

In another aspect, the present disclosure provides a computer-implemented method for predicting a risk of a liver disease of a subject, comprising: (a) receiving clinical health data of the subject, wherein the clinical health data comprises a plurality of quantitative or categorical measures of said subject; (b) using a trained algorithm to process the clinical health data of the subject to determine a risk score indicative of the risk of the liver disease of the subject; and (c) electronically outputting a report indicative of the risk score indicative of the risk of the liver disease of the subject.

In some embodiments, for example, the clinical health data comprises one or more quantitative measures of the subject, such as age, weight, height, body mass index (BMI), blood pressure, heart rate, and glucose levels. As another example, the clinical health data can comprise one or more categorical measures, such as race, ethnicity, history of disease, history of medication or other clinical treatment, history of tobacco use, history of alcohol consumption, daily activity or fitness level, genetic test results, blood test results, and imaging results.

In some embodiments, the computer-implemented method for predicting a risk of a liver disease of a subject is performed using a computer or mobile device application. For example, a subject can use a computer or mobile device application to input the subject's own clinical health data, including quantitative and/or categorical measures. The computer or mobile device application can then use a trained algorithm to process the clinical health data to determine a risk score indicative of the risk of the liver disease of the subject. The computer or mobile device application can then display a report indicative of the risk score indicative of the risk of the liver disease of the subject.

In some embodiments, the risk score indicative of the risk of the liver disease of the subject can be refined by performing one or more subsequent clinical tests for the subject. For example, the subject can be referred by a physician for one or more subsequent clinical tests (e.g., an imaging test or a blood test) based on the initial risk score. Next, the computer or mobile device application may process results from the one or more subsequent clinical tests using a trained algorithm to determine an updated risk score indicative of the risk of the liver disease of the subject.

In some embodiments, the risk score comprises a likelihood of the subject having a liver disease within a pre-determined duration of time. For example, the pre-determined duration of time may be about 1 hour, about 2 hours, about 4 hours, about 6 hours, about 8 hours, about 10 hours, about 12 hours, about 14 hours, about 16 hours, about 18 hours, about 20 hours, about 22 hours, about 24 hours, about 1.5 days, about 2 days, about 2.5 days, about 3 days, about 3.5 days, about 4 days, about 4.5 days, about 5 days, about 5.5 days, about 6 days, about 6.5 days, about 7 days, about 8 days, about 9 days, about 10 days, about 12 days, about 14 days, about 3 weeks, about 4 weeks, about 5 weeks, about 6 weeks, about 7 weeks, about 8 weeks, about 9 weeks, about 10 weeks, about 11 weeks, about 12 weeks, about 5 months, about 6 months, about 7 months, about 8 months, about 9 months, about 10 months, about 11 months, about 1 year, about 2 years about 3 years, about 4 years, about 5 years, or more than about 5 years.

After the liver disease state is identified or an increased risk of the liver disease is monitored in the subject, a report may be electronically outputted that is indicative of (e.g., identifies or provides an indication of) the liver disease of the subject. The subject may not display a liver disease (e.g., is asymptomatic of the liver disease). The report may be presented on a graphical user interface (GUI) of an electronic device of a user. The user may be the subject, a caretaker, a physician, a nurse, or another health care practitioner.

The report may include one or more clinical indications such as (i) a diagnosis of the liver disease of the subject, (ii) a prognosis of the liver disease of the subject, (iii) an increased risk of the liver disease of the subject, (iv) a decreased risk of the liver disease of the subject, (v) an efficacy of the course of treatment for treating the liver disease of the subject, and (vi) a non-efficacy of the course of treatment for treating the liver disease of the subject. The report may include one or more clinical actions or decisions made based on these one or more clinical indications. Such clinical actions or decisions may be directed to therapeutic interventions, induction or inhibition of labor, or further clinical assessment or testing of the liver disease of the subject.

For example, a clinical indication of a diagnosis of the liver disease of the subject may be accompanied with a clinical action of prescribing a new therapeutic intervention for the subject. As another example, a clinical indication of an increased risk of the liver disease of the subject may be accompanied with a clinical action of prescribing a new therapeutic intervention or switching therapeutic interventions (e.g., ending a current treatment and prescribing a new treatment) for the subject. As another example, a clinical indication of a decreased risk of the liver disease of the subject may be accompanied with a clinical action of continuing or ending a current therapeutic intervention for the subject. As another example, a clinical indication of an efficacy of the course of treatment for treating the liver disease of the subject may be accompanied with a clinical action of continuing or ending a current therapeutic intervention for the subject. As another example, a clinical indication of a non-efficacy of the course of treatment for treating the liver disease of the subject may be accompanied with a clinical action of ending a current therapeutic intervention and/or switching to (e.g., prescribing) a different new therapeutic intervention for the subject.

### Computer Systems

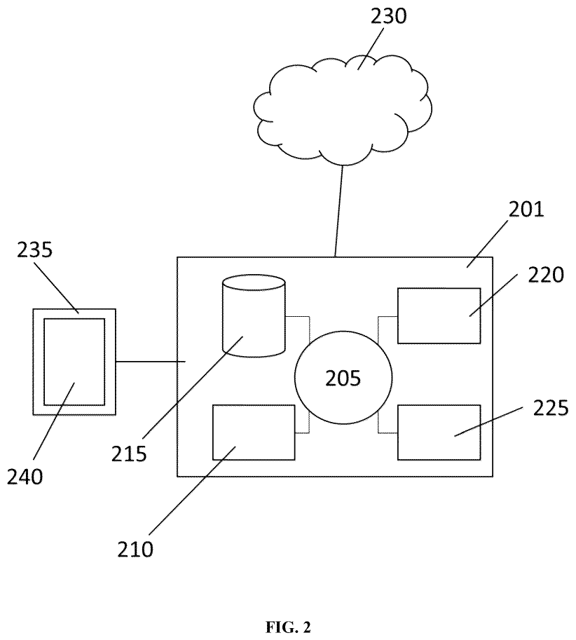

The present disclosure provides computer systems that are programmed to implement methods of the disclosure. FIG. 2 shows a computer system 201 that is programmed or otherwise configured to, for example, (i) train and test a trained algorithm, (ii) use the trained algorithm to process data to determine a liver disease state of a subject, (iii) determine a quantitative measure indicative of a liver disease state of a subject, (iv) identify or monitor the liver disease state of the subject, and (v) electronically output a report that indicative of the liver disease state of the subject.

The computer system 201 can regulate various aspects of analysis, calculation, and generation of the present disclosure, such as, for example, (i) training and testing a trained algorithm, (ii) using the trained algorithm to process data to determine a liver disease state of a subject, (iii) determining a quantitative measure indicative of a liver disease state of a subject, (iv) identifying or monitoring the liver disease state of the subject, and (v) electronically outputting a report that indicative of the liver disease state of the subject. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi-core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220, and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet.

The network 230 in some cases is a telecommunication and/or data network. The network 230 can include one or more computer servers, which can enable distributed computing, such as cloud computing. For example, one or more computer servers may enable cloud computing over the network 230 (“the cloud”) to perform various aspects of analysis, calculation, and generation of the present disclosure, such as, for example, (i) training and testing a trained algorithm, (ii) using the trained algorithm to process data to determine a liver disease state of a subject, (iii) determining a quantitative measure indicative of a liver disease state of a subject, (iv) identifying or monitoring the liver disease state of the subject, and (v) electronically outputting a report that indicative of the liver disease state of the subject. Such cloud computing may be provided by cloud computing platforms such as, for example, Amazon Web Services (AWS), Microsoft Azure, Google Cloud Platform, and IBM cloud.

The network 230, in some cases, with the aid of the computer system 201, can implement a peer-to-peer network, which may enable devices coupled to the computer system 201 to behave as a client or a server.

The CPU 205 may comprise one or more computer processors and/or one or more graphics processing units (GPUs). The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries, and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, (i) a visual display indicative of training and testing of a trained algorithm, (ii) a visual display of data indicative of a liver disease state of a subject, (iii) a quantitative measure of a liver disease state of a subject, (iv) an identification of a subject as having a liver disease state, or (v) an electronic report indicative of the liver disease state of the subject. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, (i) train and test a trained algorithm, (ii) use the trained algorithm to process data to determine a liver disease state of a subject, (iii) determine a quantitative measure indicative of a liver disease state of a subject, (iv) identify or monitor the liver disease state of the subject, and (v) electronically output a report that indicative of the liver disease state of the subject.

cfDNA Methylation

In some embodiments, cfDNA methylation data obtained from a biological sample (observation) include a set of sequenced DNA fragments that have been subjected to conversion conditions such that unmethylated cytosine sites are converted to thymine to provide methylation status of cytosine sites in the DNA fragments. Each DNA fragment may consist of a number of base-pair reads with some indicating whether a methylation site is methylated or unmethylated. Provided herein are machine learning models and systems useful for inferring relevant outcomes from such cfDNA methylation data. Non-limiting examples of such outcomes include: (i) presence or absence of a disease; (ii) type or subtype of a disease; (iii) type, dose, or a combination of treatment for treatment of a disease; (iv) predicted response of a subject to a treatment of a disease; (v) risk of a subject developing an advanced form of a disease; and (vi) outcome for the subject (prognosis).

A dataset may include cfDNA methylation data from one or more subjects, at least some of which having one or more labels described herein. A challenge in ML model training is using a dataset to produce a model that can infer outcome from a new, previously untrained cfDNA methylation data. In cfDNA methylation data, each fragment may be assigned to a location in the genome. ML models may represent data by data representation, featurization, or feature engineering. For large datasets, e.g., having millions of data points, data representation may be generated in a purely data driven manner using deep neural networks. Such networks may be designed to build a complex underlying dataset without strong assumptions purely from the data. However, in cases in which the sample size is small to moderate, e.g., cfDNA methylation data, inferring outcomes using purely data driven representation without any assumptions may be challenging. Provided herein are methods of representing data having high dimension and small sample size in a ML model for inferring outcomes with high accuracy and sensitivity. The methods described herein comprises providing a compact probability distribution of a plurality of fragments; using the compact probability distribution in intermediate training to provide a trained model; and using the trained model to featurize the plurality of fragments.

cfDNA methylation data consist of a large number of fragments; however, those fragments may originate from anywhere in the genome and samples may have different numbers of fragments. These non-uniform sparse data may also pose a challenge for ML training methods. Further, there are about 28 million methylation sites in the human genome, which is several orders of magnitude greater than the largest feasible clinical studies using cfDNA methylation data. Training on data with input dimensions having several orders of magnitude larger than the number of training data may be a challenge for ML training methods.

DNA data, including cfDNA methylation data, may be produced using sequencers, which may be an expensive and time-consuming process. ML training methods may be used to circumvent these shortcomings by leveraging data from different studies regardless of acquisition methods and data sources. Such data sources may include, but are not limited to:
cfDNA and non-cfDNA, e.g., combining data obtained from cfDNA with data obtained from tissue samples;
Different methylation assays, e.g., combining data obtained from bisulfite conversion with data obtained from enzymatic conversion assays; and
Different sequencing methods, e.g., combining data obtained from microarrays with data obtained from next generation sequencing.

Such flexibility may allow the usage of pre-acquired data, such as publicly-available data.

cfDNA methylation data may be very large given that there are around 3.2 billion genomic locations, around 28 million of which may be subject to methylation. Each fragment in cfDNA on average may have around 150 base pairs. Thus, a cfDNA methylation dataset, for example, at 30× sequencing depth for a given sample may require at least 48 gigabytes and 250 megabytes of storage for base pairs and methylation states, respectively. A training procedure containing 500 samples may require several rounds of processing. There is a need for a ML training method capable of processing such large data sets.

Distributing the training over a cluster of computers may help overcome these challenges. However, this method may have various shortcomings. Because the multiple computers need to communicate with one another during training, training may be extremely slow and time-consuming. For example, training a model with all fragments on 1,000 observations may require around 1,500 core hours and thousands of computers. Alternatively, data can be divided, e.g., by different regions in the genome, and independently processed. However, this method may prevent the ML model from learning nuance interactions between different genomic regions.

Provided herein are ML methods that alleviate the challenges described herein. A method of the disclosure comprises providing a probability distribution based on the cfDNA methylation data of a set of fragments from a biological sample; and training the probability distribution on a ML model. Instead of training on a set of fragments, the method comprises training on a probability distribution of the set of fragments. The probability distribution may represent a state of the sample; the list of observed fragments may be a draw from such probability distribution mediated by blood sampling and sequencing of the set of DNA fragments. Specifically, methods of the disclosure comprise transforming a set of input fragments into a probability distribution that is most likely to generate the input fragments.

There are various advantages in representation of data by a probability distribution. Probability distributions may not be sparse and have a predefined fixed complexity. Probability distributions may represent a likelihood of observing different methylation patterns. The probability distribution represents the state of the methylation patterns, and thus, is less susceptible to variation in assaying, sequencing methodologies, and other factors. Such characteristic may be desirable because of the availability of sequencing data in the public domain, e.g., from the National Institutes of Health and other research institutes. Further, a probability distribution is much smaller in size, and thus, may be much easier to use in the training or distributed systems. In turn, building complex models may be more feasible. If the computations are expensive, then building complex models can be prohibitively expensive and time consuming. Probability distribution may therefore provide a simpler approach that makes training a complex model feasible. Additionally, the probability distribution representing a given sample may be calculated without a need or knowledge of other samples (e.g., training other samples). Thus, the procedure may be easily distributed over a computer cluster. The procedure does not leak information between samples, and thus, may be freely performed without the need for cross-validation or on training and test datasets. Such representation may also be suitable for building a model that produces high quality inference.

Cell-free DNA methylation data may be derived from a large number of cells across the body. Assuming that each cell has a number of characteristics (Z), a cell can be represented by a mixture of those characteristics. A sample can be represented as a proportion of different cells, and thus, a proportion of such hidden characteristics. Thus, the first task is to determine the best Z characteristics from a dataset of a model from a set of probability distributions that can estimate those characteristics for a set of fragments from a cfDNA methylation dataset.

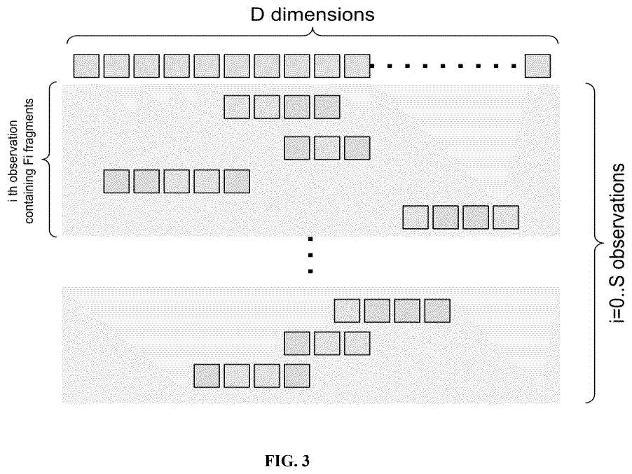

FIG. 3 illustrates a schematic of an example training dataset. From a mathematical perspective, the underlying dataset is a random variable of D dimensions (i.e., number of methylation sites) that is partially observed. Each observation (i.e., a participant) consists of a number of fragments. A fragment corresponds to a set of values corresponding to a portion of the D-dimensional space.

As described herein, the observations can be formulated as a distribution in D-dimensional space characterized by ϕs (one for each observation) instead of as a set of fragments. The parameters of the distribution, ϕₛ, are statistics of the sets of fragments. For a large class of distributions, such as exponential family, the parameters of the distribution (ϕₛ) can be explicitly represented as their sufficient statistics. For others, in a general case, the parameters of the distribution can be represented by a near sufficient statistics. For those general cases, ϕₛ can be calculated by maximizing likelihood for a class of distributions using the following equation:

$${ϕ}_{s} = arg max ( {\sum }_{i = 0}^{{F}^{( s )}} log ( p ( {f}_{i} ; {ϕ}_{s} ) ) )$$

Such probability distributions may be characterized in several ways. For example, the probability distribution representation of the sample may be represented using a Markov Model in which the probability of observing a methylation state is dependent on its genomic location as well as the state of the previous methylation sites. Such a model may be made by quantifying the number of observed states as well as the number of k-mers at each genomic location or methylation site, which can be determined using the following equation:

$$f = \{ {s}_{i} ; i ∈ ( a , b ) a < b < D \} , p ( f ; ϕ ) = p ( {s}_{a} ; ϕ ) {\sum }_{i = a + 1}^{b} p ( {s}_{i} ; ϕ )$$

where s is the state of the k-mer at a particular location.

Assuming all the data are represented as the parameters of probability distributions (i.e., estimated all ϕᵢ for all observations), several approaches may be used to estimate the mentioned hidden Z characteristics. One approach involves maximizing the likelihood using the following equation:

$$l ( θ ) = {\sum }_{i = 0}^{S} log ( p ( {ϕ}_{i} ; θ ) ) = {\sum }_{i = 0}^{S} log {\sum }_{z} p ( {ϕ}_{i} . z ; θ ) = {\sum }_{i = 0}^{S} log {\sum }_{z} {q}_{i , z} p ( {ϕ}_{i} ; {θ}_{z} )$$

where θ_{z} is a distribution over D, similar to ϕ used to describe a characteristic. Such likelihood may be maximized using the expectation maximization equation below:

$$Expectation : {q}_{i , z} = arg max ( - {\vert {ϕ}_{i} - {\sum }_{z} {q}_{i , z} {θ}_{z} \vert}_{2} )$$

In the Expectation step, based on the current estimation of θ, the most likely q_{i,z} can be determined.

$$Maximization : θ = arg max ( {\sum }_{i = 0}^{S} {\sum }_{z} {q}_{i , z} log \frac{p ( {ϕ}_{i} ; {θ}_{z} )}{{q}_{i , z}} )$$

In the Maximization step, based on the current estimation of q, the most likely θ can be determined.

The output of the above method is a set of Z parameters (θ) describing the hidden characteristics of the dataset.

These estimations may not rely on a distribution assumption, such as Gaussian or Bernoulli distributions.

Since the data size is substantially reduced because of this specific representation of the data, most calculations may be processed on a general purpose computer or be easily distributed across a plurality of computers for faster runtime.

The outcome of the first operation is the representative distribution corresponding to the unknown Z characteristics. These characteristics do not need to be known in advance or assigned by experts.

Since this first operation may be used to estimate a set of biological characteristics, data may be incorporated and/or aggregated from various sources, including cfDNA data, data from different assays (e.g., RNA data, proteomic data, metabolomics data, etc.), data with different sequencing depths, and/or data generated from different sequencing methodologies.

Given a set of Z characteristics (representative distributions), a set of fragments may be converted into a fixed set of features in several ways. For example, an observation may be represented as a histogram over location and the above characteristics. A Z×D zero matrix may be used as a starting point. For each fragment, the Z×1 vector may be incremented at the location of the fragment within D using the following equation:

$$p ( f ; {θ}_{z} ) = p ( {s}_{a} ; {θ}_{z} ) {\sum }_{i = a + 1}^{b} p ( {s}_{i} ; {θ}_{z} )$$

For each Z components.

Alternatively, or in addition, fragments may be represented by how informative the fragments are in relation to the characteristics. For example, a probability of observing a fragment in an observation may be determined using the following equation:

FragFreq=p(f;ϕᵢ)

The proportion of the characteristics that is expected to produce fragment f to the total number of characteristics may be determined using the following equation:

$$InverseSampleFreq = \frac{\{ E ( f ∼ {θ}_{i} ) > 1 / N \}}{Z}$$

Each fragment may then be represented as Z+1 number corresponding to FragFreq×InverseSampleFreq for the observation ϕᵢ and Z characteristics.

Once the observation is represented as a fixed size, these representations may be additive. Thus:
The representation from two sets of fragments is equal to the representation of each set added together.
Representation can be reduced from Z×D to Z×A by adding D÷A columns of the matrix together.

The set of fragments is used only once in the above representation and may be calculated based only on known θ parameters. As such, the method overcomes the challenges described herein. Because probability distribution may provide a smaller and more biologically accurate representation of a sample, the ML method described above does not require fragmentation of the genome into small regions in order for the method to be computationally feasible.

### EXAMPLES

### Example 1: Classification of Liver Disease Using Methylation Data from Patient Plasma Samples

Plasma samples were collected from individual patients previously diagnosed with various liver diseases, including non-alcoholic fatty liver disease (NAFLD), non-alcoholic steatohepatitis (NASH), and cirrhosis. The methodologies described above were used to determine the methylation pattern of DNA across the entire genome. Firstly, cell-free DNA (cfDNA) was extracted from a biological sample, e.g., plasma isolated from blood. The extracted DNA was then treated with sodium bisulfite to convert unmethylated cytosines to uracil, while methylated cytosines remain unchanged. The bisulfite-treated DNA was then subjected to library preparation including end repair and A-tailing, where the DNA ends were blunted, and an adenine nucleotide was added to the 3′ end of each strand. Following this, specific adapters were ligated to the ends of the DNA to enable the DNA to bind to the sequencing platform and provide sites for primer binding during amplification. The adapter-ligated DNA was then subjected to PCR amplification. The amplified DNA was sequenced using high-throughput DNA sequencing technologies to determine the methylation patterns of the DNA molecules within the cfDNA samples, resulting in the generation of approximately 500 million cfDNA reads that have information about approximately 28 million CpGs.

Additionally, independent data derived from methylation microarrays were utilized to generate characteristics described using the methods above. These microarrays included data from a multitude of cell types such as liver, brain, and heart cells, in both healthy and diseased states. This approach excluded the use of labels indicating the cell type or condition and relied solely on the methylation microarray data.

The methylation data were computer-processed to generate a set of three characteristics (Z=3) with distribution within the exponential family.

For each plasma sample (each comprising approximately 500 million cfDNA reads), the cfDNA was converted into a fixed set of features using the generated characteristics. cfDNA fragments were mapped to specific genomic locations and then the fragments were converted to Z=3 features one at each characteristic. The fragment frequency and inverse sample frequency were then calculated for each fragment and another feature was calculated as fragment frequency times inverse sample frequency to have 3+1=4 features per fragment.

The feature of each fragment was added to the CpG location of its first CpG to finally convert the whole sample to 4 By approximately 28 million features.

This process was further enhanced by the additive feature as described above to further reduce the dimensionality of the sample representation from 4 by approximately 28 million to a 4 by 100 totaling 4*100-400 features.

While various machine learning training methodologies may be applicable to these representations, a simplified approach using 1-nearest neighbor classifier was employed to demonstrate the efficacy of the disclosed methods. Using the independent microarray data, an average representation for liver disease was computed, and a score was calculated for each sample, indicating the distance between the sample and the average liver disease representation.

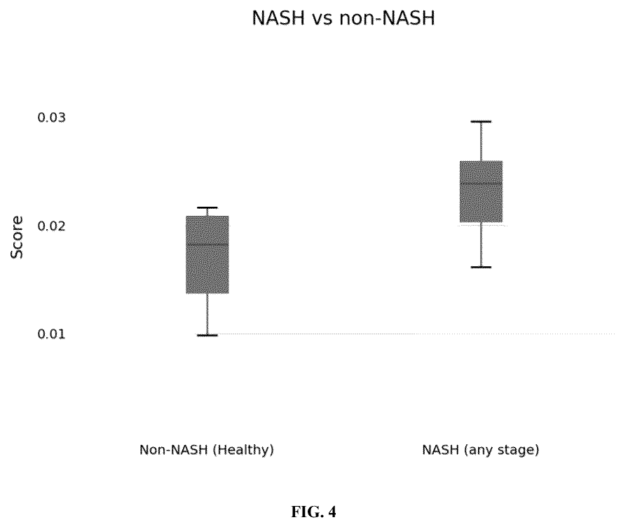

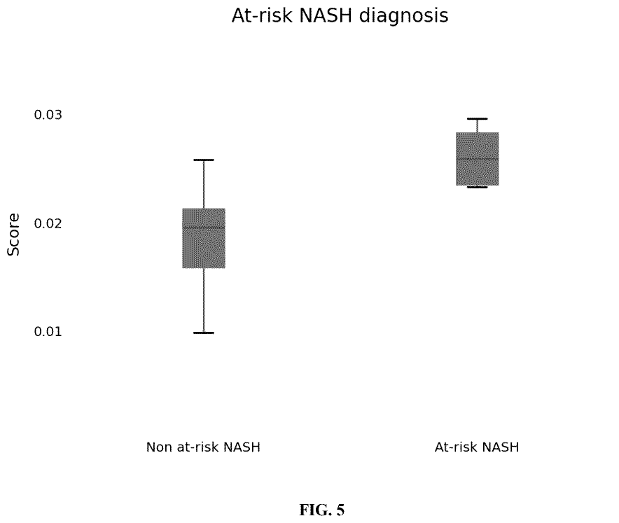

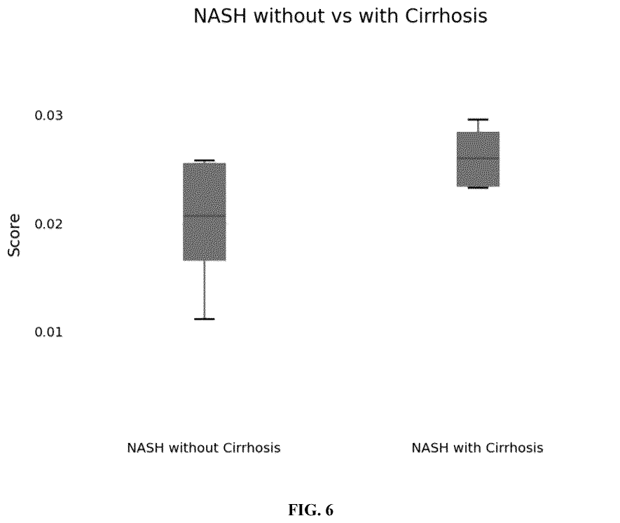

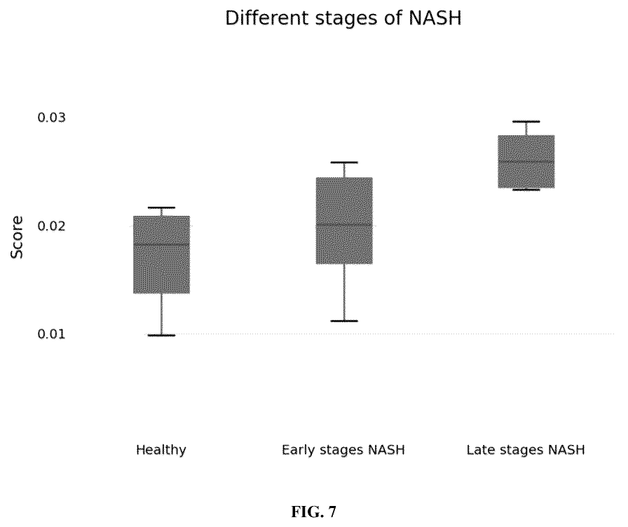

The methods were repeated for several applications, including for distinguishing NASH from non-NASH (healthy) samples (FIG. 4), distinguishing at-risk NASH from non-at-risk NASH samples, with at-risk NASH defined as individuals with NASH and stage 2 fibrosis or higher (FIG. 5), distinguishing NASH samples with or without cirrhosis (FIG. 6), and distinguishing early stage NASH, late stage NASH, and non-NASH (healthy) samples (FIG. 7).

The results shown in FIG. 4 and FIG. 6 demonstrate that the disclosed methods can be used to identify subjects with liver conditions. FIG. 4 shows the identification of NASH and FIG. 6 shows the identification of cirrhosis. FIG. 5 shows the disclosed methods can also be used to stratify subjects with liver condition based on prognosis. FIG. 7 shows the disclosed methods can be used to differentiate between early stage and late stage liver disease.

What is claimed is:

1. A method comprising:
(a) providing a cell-free deoxyribonucleic acid (cfDNA) sample derived from a subject; and
(b) sequencing the cfDNA sample or a derivative thereof to determine a methylation pattern or a methylation level of DNA molecules of the cfDNA sample.

2. The method of claim 1, further comprising, prior to the sequencing, processing the DNA molecules of the cfDNA sample with a reaction mixture comprising enzymes for methylation-aware sequencing.

3. The method of claim 1, further comprising, prior to the sequencing, processing the DNA molecules of the cfDNA sample with a reaction mixture comprising bisulfite.

4. The method of claim 1, wherein the cfDNA sample is obtained or derived from a plasma sample.

5. The method of claim 1, wherein the cfDNA sample is obtained or derived from a serum sample.

6. The method of claim 1, wherein the cfDNA sample is obtained or derived from a urine sample.

7. The method of claim 1, wherein the cfDNA sample is obtained or derived from a saliva sample.

8. The method of claim 1, wherein the cfDNA sample is obtained or derived from a liver tissue sample.

9. The method of claim 1, further comprising fractionating a whole blood sample derived from the subject to provide the cfDNA sample.

10. The method of claim 9, wherein the fractionating comprises centrifugation.

11. The method of claim 1, further comprising performing amplification of nucleic acid molecules obtained or derived from the cfDNA sample.

12. The method of claim 11, wherein the amplification comprises polymerase chain reaction (PCR).

13. The method of claim 1, wherein (a) comprises subjecting the cfDNA sample to conditions that are sufficient to isolate, enrich, or extract a set of DNA molecules, and wherein (b) comprises sequencing DNA molecules derived from the set of DNA molecules.

14. The method of claim 13, wherein (b) comprises using nucleic acid primers to selectively enrich the set of DNA molecules.

15. The method of claim 13, wherein (b) comprises using nucleic acid probes to selectively enrich the set of DNA molecules.

16. The method of claim 1, wherein the method does not comprise nucleic acid isolation, enrichment, or extraction.
